SEQUENCE LISTING (146 sequences)

SEQ ID NO: 1 - DNA, 798 bp, Saccharomyces cerevisiae
  misc_feature (1)..(798): HAP2 nucleotide
atgtcagcagacgaaacggatgcgaaatttcatccattagaaacagatctgcaatctgatacagcggctgcaacatcaacggcagcagcttcacgcagtccctctcttcaagagaagcccatagagatgcccttggatatgggaaaagcgccttctccaagaggcgaagatcaacgggttacaaatgaagaagatttgtttttgtttaacagattgcgggcatcacagaatagagttatggactccttggaaccacaacaacagtcacagtatacatcttccagtgtcagtacgatggaaccatctgccgactttactagtttctctgcagtgactactttaccgcctcctcctcatcaacaacaacagcaacaacagcagcagcagcagcagcagcaattggtggttcaagcccagtacacccaaaatcaaccaaacttgcaaagcgatgttttaggaaccgctatagcagagcaaccattttatgttaatgccaagcagtactaccgaattttgaaaaggcgatatgcaagagctaaactagaggaaaagctacgaatatcaagagaacgaaagccatacttacacgaatctcgacataaacatgcgatgcgaagacctcgtggtgaaggtgggaggttcttgacagccgctgagatcaaagccatgaaatcgaagaaaagtggggctagcgatgatcctgacgatagtcatgaggataaaaaaatcactactaaaataatacaagaacagccgcatgctacttccaccgcagctgcagcagacaaaaaaacataa

SEQ ID NO: 2 - protein, 265 aa, Saccharomyces cerevisiae
  misc_feature (1)..(265): Hap2p amino acid
MSADETDAKFHPLETDLQSDTAAATSTAAASRSPSLQEKPIEMPLDMGKAPSPRGEDQRVTNEEDLFLFNRLRASQNRVMDSLEPQQQSQYTSSSVSTMEPSADFTSFSAVTTLPPPPHQQQQQQQQQQQQQQLVVQAQYTQNQPNLQSDVLGTAIAEQPFYVNAKQYYRILKRRYARAKLEEKLRISRERKPYLHESRHKHAMRRPRGEGGRFLTAAEIKAMKSKKSGASDDPDDSHEDKKITTKIIQEQPHATSTAAAADKKT

SEQ ID NO: 3 - DNA, 435 bp, Saccharomyces cerevisiae
  misc_feature (1)..(435): HAP3 nucleotide
atgaataccaacgagtccgaacatgttagcacaagcccagaggatactcaggagaacggtggaaacgctagctccagcggcagtttgcagcaaatttccacgctaagagagcaggacagatggctacccatcaacaatgtagcgcgactcatgaagaatactctcccaccgagtgctaaggtatcgaaagatgcgaaagagtgcatgcaggagtgtgtcagtgagctcatttcttttgtgactagcgaggccagcgatcgatgcgctgctgacaaaagaaagacgataaacggggaagacattctcatatcattgcacgccttaggattcgagaactatgcagaggtgttgaaaatctacttggctaaatacaggcaacaacaggcgctgaagaatcaactaatgtatgagcaggacgacgaagaggtgccttga

SEQ ID NO: 4 - protein, 144 aa, Saccharomyces cerevisiae [sequence omitted]

SEQ ID NO: 5 - DNA, 1665 bp, Saccharomyces cerevisiae
  misc_feature (1)..(1665): HAP4 nucleotide
atgaccgcaaagacttttctactacaggcctccgctagtcgccctcgtagtaaccattttaaaaatgagcataataatattccattggcgcctgtaccgatcgccccaaataccaaccatcataacaatagttcgctggaattcgaaaacgatggcagtaaaaagaagaagaagtctagcttggtggttagaacttcaaaacattgggttttgcccccaagaccaagacctggtagaagatcatcttctcacaacactctacctgccaacaacaccaataatattttaaatgttggccctaacagcaggaacagtagtaataataataataataataacatcatttcgaataggaaacaagcttccaaagaaaagaggaaaataccaagacatatccagacaatcgatgaaaagctaataaacgactcgaattacctcgcatttttgaagttcgatgacttggaaaatgaaaagtttcattcttctgcctcctccatttcatctccatcttattcatctccatctttttcaagttatagaaatagaaaaaaatcagaattcatggacgatgaaagctgcaccgatgtggaaaccattgctgctcacaacagtctgctaacaaaaaaccatcatatagattcttcttcaaatgttcacgcaccacccacgaaaaaatcaaagttgaacgactttgatttattgtccttatcttccacatcttcatcggccactccggtcccacagttgacaaaagatttgaacatgaacctaaattttcataagatccctcataaggcttcattccctgattctccagcagatttctctccagcagattcagtctcgttgattagaaaccactccttgcctactaatttgcaagttaaggacaaaattgaggatttgaacgagattaaattctttaacgatttcgagaaacttgagtttttcaataagtatgccaaagtcaacacgaataacgacgttaacgaaaataatgatctctggaattcttacttacagtctatggacgatacaacaggtaagaacagtggcaattaccaacaagtggacaatgacgataatatgtctttattgaatctgccaattttggaggaaaccgtatcttcagggcaagatgataaggttgagccagatgaagaagacatttggaattatttaccaagttcaagttcacaacaagaagattcatcacgtgctttgaaaaaaaatactaattctgagaaggcgaacatccaagcaaagaacgatgaaacctatctgtttcttcaggatcaggatgaaagcgctgattcgcatcaccatgacgagttaggttcagaaatcactttggctgacaataagttttcttatttgcccccaactctagaagagttgatggaagagcaggactgtaacaatggcagatcttttaaaaatttcatgttttccaacgataccggtattgacggtagtgccggtactgatgacgactacaccaaagttctgaaatccaaaaaaatttctacgtcgaagtcgaacgctaacctttatgacttaaacgataacaacaatgatgcaactgccaccaatgaacttgatcaaagcagtttcatcgacgaccttgacgaagatgtcgattttttaaaggtacaagtattttga

SEQ ID NO: 6 - protein, 554 aa, Saccharomyces cerevisiae
  misc_feature (1)..(554): Hap4p amino acid
MTAKTFLLQASASRPRSNHFKNEHNNIPLAPVPIAPNTNHHNNSSLEFENDGSKKKKKSSLVVRTSKHWVLPPRPRPGRRSSSHNTLPANNTNNILNVGPNSRNSSNNNNNNNIISNRKQASKEKRKIPRHIQTIDEKLINDSNYLAFLKFDDLENEKFHSSASSISSPSYSSPSFSSYRNRKKSEFMDDESCTDVETIAAHNSLLTKNHHIDSSSNVHAPPTKKSKLNDFDLLSLSSTSSSATPVPQLTKDLNMNLNFHKIPHKASFPDSPADFSPADSVSLIRNHSLPTNLQVKDKIEDLNEIKFFNDFEKLEFFNKYAKVNTNNDVNENNDLWNSYLQSMDDTTGKNSGNYQQVDNDDNMSLLNLPILEETVSSGQDDKVEPDEEDIWNYLPSSSSQQEDSSRALKKNTNSEKANIQAKNDETYLFLQDQDESADSHHHDELGSEITLADNKFSYLPPTLEELMEEQDCNNGRSFKNFMFSNDTGIDGSAGTDDDYTKVLKSKKISTSKSNANLYDLNDNNNDATATNELDQSSFIDDLDEDVDFLKVQVF

SEQ ID NO: 7 - DNA, 729 bp, Saccharomyces cerevisiae
  misc_feature (1)..(729): HAP5 nucleotide
atgactgataggaatttctcaccacaacaaggacaaggacctcaagaatcgctcccggagggaccgcaacccagtacgatgattcagagagaggaaatgaatatgccaaggcaatattcagaacagcaacaactgcaagaaaacgaaggggaaggggaaaatacgaggctacctgtttctgaggaagagttccggatggtacaggagttgcaagctatccaggcgggccatgaccaagctaatctaccgccaagtggtcgaggatcgcttgaaggcgaagataatggaaacagcgacggcgcagacggagaaatggacgaggacgatgaagagtatgatgtgtttaggaacgttggtcagggattggtgggccactacaaggagataatgatccgttattggcaagaattaatcaacgagatcgagtctacgaatgaacctggttccgagcatcaagatgacttcaaatcacattccttaccatttgcgagaatccgcaaggtcatgaagacggatgaagatgtcaagatgattagtgcagaggcccccatcattttcgccaaagcctgtgagatctttattacagaactgactatgagagcttggtgcgtggcagaaaggaataaaagacgaactttgcagaaggcagatatcgcagaggccctgcaaaagagtgacatgtttgactttctcatcgatgttgtgcctagaagacctcttccacaatga

SEQ ID NO: 8 - protein, 242 aa, Saccharomyces cerevisiae
  misc_feature (1)..(242): Hap5p amino acid
MTDRNFSPQQGQGPQESLPEGPQPSTMIQREEMNMPRQYSEQQQLQENEGEGENTRLPVSEEEFRMVQELQAIQAGHDQANLPPSGRGSLEGEDNGNSDGADGEMDEDDEEYDVFRNVGQGLVGHYKEIMIRYWQELINEIESTNEPGSEHQDDFKSHSLPFARIRKVMKTDEDVKMISAEAPIIFAKACEIFITELTMRAWCVAERNKRRTLQKADIAEALQKSDMFDFLIDVVPRRPLPQ

SEQ ID NO: 9 - DNA, 1000 bp, Saccharomyces cerevisiae
aatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtgggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacaca

SEQ ID NO: 10 - DNA, 1000 bp, Saccharomyces cerevisiae
gaaaacaccggcaacaagctattaaggagggtgaaaagcttgaaaactagtaaaaagcactgatcaatgcttctttcttgcgttctttttgttgcgcccatatattattttatttattacattcatatagcaatatttaccttattttattggttacttttctatacgcaaaatcactacactatgttatgttaaggtctccgatacgggaatataccaatcaatacttatcacttcggattttttatgggtcttatccccactgttccattttcttgtttaaggcatcccggaggataaactaaaaaggtggcccatcccacccgaaatgaaagtaatcatctgctagcaaaaagtaaagaaatgagagcatgctgtgatgtactggtggacgaaattgtgacccatacccaccgaagaaacatccgcatgacgtgttactgttacttcccggattaagggatgcattctaactctgtgcgcccttttctctgcagttgatccgcattccccgtggctgtgcacattaggggacagtaagtaattcactttctgatcccgcactcatagcgatggaataatataccggatttcacaccttgttattgagtgaagtactgcttggtgaaatgatatctttatgttcaatattaatggtcgtgtggatgaatatatgggcatgggttaattagttttaggggcacggagtaaacaagaaaggagggccagaatcattagtagagtacctcaagtttggtttctttttgatttcacgtataaaagagtctctctcttttcttttcatgctagtcgaacggttctccctctaagaataagaaactatcaaaagaaagagaaaagtcgattgaataatttttctatatataatatacgcaaacaagattcgctttcactttgcaattttacttcatagctttgttaaaaccagcaaaaaatattatttttctagaaaaaagaatatattagaggtaaagaaaga

SEQ ID NO: 11 - DNA, 1000 bp, Saccharomyces cerevisiae
aatagtactctcatcgctaagatcatttggggttgttaagcatgccctgctaaacacgccctactaaacacttcaaaagcaacttaaaatatttttatctaattatagctaaaacccaatgtgaaagacatatcatactgtaaaagtgaaaaagcagcaccgttgaacgccgcaagagtgctcccataacgctttactagagggctagattttaatggccccttcatggagaagttatgaggacaaatcccactacagaaagcgcaacaaatttttttttccgtaacaacaaacatctcatctagtttctgccttaaacaaagccgcagccagagccgtttttccgccatatttatccaggattgttccatacggctccgtcagaggctgctacgggatgttttttttttaccccgtggaaatgaggggtatgcaggaatttgtgcggggtaggaaatcttttttttttttaggaggaacaactggtggaagaatgcccacacttctcagaaatgcatgcagtggcagcacgctaattcgaaaaaattctccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 12 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 13 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 14 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 15 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 16 - DNA, 2055 bp, Klebsiella pneumoniae
tcgaccacggggtgctgaccttcggcgaaattcacaagctgatgatcgacctgcccgccgacagcgcgttcctgcaggctaatctgcatcccgataatctcgatgccgccatccgttccgtagaaagttaagggggtcacatggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaagtcatcacaataaggaaagaaaaatgaaaaaagtcgcacttgttaccggcgccggccaggggattggtaaagctatcgcccttcgtctggtgaaggatggatttgccgtggccattgccgattataacgacgccaccgccaaagcggtcgcctccgaaatcaaccaggccggcggccgcgccatggcggtgaaagtggatgtttctgaccgcgaccaggtatttgccgccgtcga

SEQ ID NO: 17 - protein, 554 aa, Lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 18 - DNA, 3220 bp, Lactococcus lactis
tagatccggaaacaactgattacctgagttaacttagcagaaattgcagaagataacggtaatttggatgaagcattaaattacctttatcaaattccggtgaatgatgaaaattatattgctgctttaatcaaaattgctgacttatatcaatttgaagttgattttgaaacagcaatttctaagttagaagaagcaagagaattatcggattctcctctgattacttttgctttggctgagtcctactttgaacaaggtgattattcagctgccattaccgaatatgcaaaactttcagaacgaaaaattttacatgaaacaaaaatttctatttatcaaagaattggtgactcttatgcccaattaggtaattttgagaatgccatatcatttcttgaaaaatcacttgaatttgatgaaaaaccggaaaccttgtataaaattgctcttctttatggagaaactcataatgaaacaagagccattgctaatttcaaacggttagaaaaaatggatgttgaatttttgaactatgaattagcctatgcccaaaccctagaagctaatcaagaatttaaagctgcactagaaatggcaaagaaagggatgaaaaaaaatcctaatgccgttcctctcttacacttcgcttcaaaaatttgtttcaaacttaaggacaaagctgcagcagaacgttatctcgtggatgctttaaatttaccagaattacatgacgaaacagtctttttgcttgctaatttatacttcaacgaagaagattttgaagctgtcattaatcttgaagagcttttagaagatgaacatttattagctaaatggctttttgcaggagcacataaagctttggaaaatgattctgaagcggctgctttgtatgaagaactcattcaaaccaatctgtcagagaatccagagtttttagaagactatattgattttcttaaagaaattggtcaaatttctaaaacagaaccaattattgaacaatatttggaacttgttccagatgatgaaaatatgagaaatttactgacagacttaaaaaataattactgacaaagctgtcagtaattatttttattgtaagctagaaaattcaaaaacttgcgtcaaaataattgtaaaaggttctattatctgataaaatgattgtgaagtaatccaagagattatgaaatatgaattagaacaaatagaggtaaaataaaaaatgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattgattacaatcaagcaatttgtggcataacaaaataaaagaagaaggccttgaacacctaagcgttcagggcctttttttgtgaaataaattagatgaaatttacaatgagttttgtgaaactagcttctagtttgtgaaaaattgcctataattgccgaataaaaatacccatttaccactccaagaggatgcttcaaattagctaaatacccgttttagaggatgcgtaaaaacaacaaaagaggatgagtatagaacgataaaacttttttatgataggttgagagaattgaatataaaatataataagtagaaggcagcaatt

SEQ ID NO: 19 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 20 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 21 - protein, 395 aa, Saccharomyces cerevisiae
MLRTQAARLICNSRVITAKRTFALATRAAAYSRPAARFVKPMITTRGLKQINFGGTVETVYERADWPREKLLDYFKNDTFALIGYGSQGYGQGLNLRDNGLNVIIGVRKDGASWKAAIEDGWVPGKNLFTVEDAIKRGSYVMNLLSDAAQSETWPAIKPLLTKGKTLYFSHGFSPVFKDLTHVEPPKDLDVILVAPKGSGRTVRSLFKEGRGINSSYAVWNDVTGKAHEKAQALAVAIGSGYVYQTTFEREVNSDLYGERGCLMGGIHGMFLAQYDVLRENGHSPSEAFNETVEEATQSLYPLIGKYGMDYMYDACSTTARRGALDWYPIFKNALKPVFQDLYESTKNGTETKRSLEFNSQPDYREKLEKELDTIRNMEIWKVGKEVRKLRPENQ

SEQ ID NO: 22 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 23 - protein, 330 aa, Methanococcus maripaludis
MKVFYDSDFKLDALKEKTIAVIGYGSQGRAQSLNMKDSGLNVVVGLRKNGASWNNAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILREIQDGRFTKEFLLEKQVSYAHLKSMRRLEGDLQIEEVGAKLRKMCGLEKEE

SEQ ID NO: 24 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatggaacaacgctaaagcagacggtcacaatgtaatgaccattgaagaagctgctgaaaaagcggacatcatccacatcttaatacctgatgaattacaggcagaagtttatgaaagccagataaaaccatacctaaaagaaggaaaaacactaagcttttcacatggttttaacatccactatggattcattgttccaccaaaaggagttaacgtggttttagttgctccaaaatcacctggaaaaatggttagaagaacatacgaagaaggtttcggtgttccaggtttaatctgtattgaaattgatgcaacaaacaacgcatttgatattgtttcagcaatggcaaaaggaatcggtttatcaagagctggagttatccagacaactttcaaagaagaaacagaaactgaccttttcggtgaacaagctgttttatgcggtggagttaccgaattaatcaaggcaggatttgaaacactcgttgaagcaggatacgcaccagaaatggcatactttgaaacctgccacgaattgaaattaatcgttgacttaatctaccaaaaaggattcaaaaacatgtggaacgatgtaagtaacactgcagaatacggcggacttacaagaagaagcagaatcgttacagctgattcaaaagctgcaatgaaagaaatcttaagagaaatccaagatggaagattcacaaaagaattccttctcgaaaaacaggtaagctatgctcatttaaaatcaatgagaagactcgaaggagacttacaaatcgaagaagtcggcgcaaaattaagaaaaatgtgcggtcttgaaaaagaagaataa

SEQ ID NO: 25 - protein, 342 aa, Bacillus subtilis
MVKVYYNGDIKENVLAGKTVAVIGYGSQGHAHALNLKESGVDVIVGVRQGKSFTQAQEDGHKVFSVKEAAAQAEIIMVLLPDEQQQKVYEAEIKDELTAGKSLVFAHGFNVHFHQIVPPADVDVFLVAPKGPGHLVRRTYEQGAGVPALFAIYQDVTGEARDKALAYAKGIGGARAGVLETTFKEETETDLFGEQAVLCGGLSALVKAGFETLTEAGYQPELAYFECLHELKLIVDLMYEEGLAGMRYSISDTAQWGDFVSGPRVVDAKVKESMKEVLKDIQNGTFAKEWIVENQVNRPRFNAINASENEHQIEVVGRKLREMMPFVKQGKKKEAVVSVAQN

SEQ ID NO: 26 - DNA, 1476 bp, Bacillus subtilis; identical to SEQ ID NO: 20

SEQ ID NO: 27 - protein, 343 aa, Anaerostipes caccae
MEECKMAKIYYQEDCNLSLLDGKTIAVIGYGSQGHAHALNAKESGCNVIIGLYEGAKEWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 28 - protein, 343 aa, Anaerostipes caccae
MEECKMAKIYYQEDCNLSLLDGKTIAVIGYGSQGHAHALNAKESGCNVIIGLYEGAKDWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 29 - protein, 343 aa, Artificial sequence
  Anaerostipes caccae KARI variant K9JB4P
MEECKMAKIYYQEDCNLSLLDGKTIAVIGYGSQGHAHALNAKESGCNVIIGLYEGAEEWKRAEEQGFEVYTAAEAAKKADIIMILIPDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 30 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 31 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 32 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 33 - DNA, 1131 bp, Saccharomyces cerevisiae
atgaccttggcacccctagacgcctccaaagttaagataactaccacacaacatgcatctaagccaaaaccgaacagtgagttagtgtttggcaagagcttcacggaccacatgttaactgcggaatggacagctgaaaaagggtggggtaccccagagattaaaccttatcaaaatctgtctttagacccttccgcggtggttttccattatgcttttgagctattcgaagggatgaaggcttacagaacggtggacaacaaaattacaatgtttcgtccagatatgaatatgaagcgcatgaataagtctgctcagagaatctgtttgccaacgttcgacccagaagagttgattaccctaattgggaaactgatccagcaagataagtgcttagttcctgaaggaaaaggttactctttatatatcaggcctacattaatcggcactacggccggtttaggggtttccacgcctgatagagccttgctatatgtcatttgctgccctgtgggtccttattacaaaactggatttaaggcggtcagactggaagccactgattatgccacaagagcttggccaggaggctgtggtgacaagaaactaggtgcaaactacgccccctgcgtcctgccacaattgcaagctgcttcaaggggttaccaacaaaatttatggctatttggtccaaataacaacattactgaagtcggcaccatgaatgcttttttcgtgtttaaagatagtaaaacgggcaagaaggaactagttactgctccactagacggtaccattttggaaggtgttactagggattccattttaaatcttgctaaagaaagactcgaaccaagtgaatggaccattagtgaacgctacttcactataggcgaagttactgagagatccaagaacggtgaactacttgaagcctttggttctggtactgctgcgattgtttctcccattaaggaaatcggctggaaaggcgaacaaattaatattccgttgttgcccggcgaacaaaccggtccattggccaaagaagttgcacaatggattaatggaatccaatatggcgagactgagcatggcaattggtcaagggttgttactgatttgaactga

SEQ ID NO: 34 - protein, 550 aa, Methanococcus maripaludis
MISDNVKKGVIRTPNRALLKACGYTDEDMEKPFIGIVNSFTEVVPGHIHLRTLSEAAKHGVYANGGTPFEFNTIGICDGIAMGHEGMKYSLPSREIIADAVESMARAHGFDGLVLIPTCDKIVPGMIMGALRLNIPFIVVTGGPMLPGEFQGKKYELISLFEGVGEYQVGKITEEELKCIEDCACSGAGSCAGLYTANSMACLTEALGLSLPMCATTHAVDAQKVRLAKKSGSKIVDMVKEDLKPTDILTKEAFENAILVDLALGGSTNTTLHIPAIANEIENKFITLDDFDRLSDEVPHIASIKPGGEHYMIDLHNAGGIPAVLNVLKEKIRDTKTVDGRSILEIAESVKYINYDVIRKVEAPVHETAGLRVLKGNLAPNGCVVKIGAVHPKMYKHDGPAKVYNSEDEAISAILGGKIVEGDVIVIRYEGPSGGPGMREMLSPTSAICGMGLDDSVALITDGRFSGGSRGPCIGHVSPEAAAGGVIAAIENGDIIKIDMIEKEINVDLDESVIKERLSKLGEFEPKIKKGYLSRYSKLVSSADEGAVLK

SEQ ID NO: 35 - DNA, 1653 bp, Methanococcus maripaludis
atgataagtgataacgtcaaaaagggagttataagaactccaaaccgagctcttttaaaggcttgcggatatacagacgaagacatggaaaaaccatttattggaattgtaaacagctttacagaagttgttcccggccacattcacttaagaacattatcagaagcggctaaacatggtgtttatgcaaacggtggaacaccatttgaatttaataccattggaatttgcgacggtattgcaatgggccacgaaggtatgaaatactctttaccttcaagagaaattattgcagacgctgttgaatcaatggcaagagcacatggatttgatggtcttgttttaattcctacgtgtgataaaatcgttcctggaatgataatgggtgctttaagactaaacattccatttattgtagttactggaggaccaatgcttcccggagaattccaaggtaaaaaatacgaacttatcagcctttttgaaggtgtcggagaataccaagttggaaaaattactgaagaagagttaaagtgcattgaagactgtgcatgttcaggtgctggaagttgtgcagggctttacactgcaaacagtatggcctgccttacagaagctttgggactctctcttccaatgtgtgcaacaacgcatgcagttgatgcccaaaaagttaggcttgctaaaaaaagtggctcaaaaattgttgatatggtaaaagaagacctaaaaccaacagacatattaacaaaagaagcttttgaaaatgctattttagttgaccttgcacttggtggatcaacaaacacaacattacacattcctgcaattgcaaatgaaattgaaaataaattcataactctcgatgactttgacaggttaagcgatgaagttccacacattgcatcaatcaaaccaggtggagaacactacatgattgatttacacaatgctggaggtattcctgcggtattgaacgttttaaaagaaaaaattagagatacaaaaacagttgatggaagaagcattttggaaatcgcagaatctgttaaatacataaattacgacgttataagaaaagtggaagctccggttcacgaaactgctggtttaagggttttaaagggaaatcttgctccaaacggttgcgttgtaaaaatcggtgcagtacatccgaaaatgtacaaacacgatggacctgcaaaagtttacaattccgaagatgaagcaatttctgcgatacttggcggaaaaattgtagaaggggacgttatagtaatcagatacgaaggaccatcaggaggccctggaatgagagaaatgctctccccaacttcagcaatctgtggaatgggtcttgatgacagcgttgcattgattactgatggaagattcagtggtggaagtaggggcccatgtatcggacacgtttctccagaagctgcagctggcggagtaattgctgcaattgaaaacggggatatcatcaaaatcgacatgattgaaaaagaaataaatgttgatttagatgaatcagtcattaaagaaagactctcaaaactgggagaatttgagcctaaaatcaaaaaaggctatttatcaagatactcaaaacttgtctcatctgctgacgaaggggcagttttaaaataa

SEQ ID NO: 36 - protein, 558 aa, Bacillus subtilis
MAELRSNMITQGIDRAPHRSLLRAAGVKEEDFGKPFIAVCNSYIDIVPGHVHLQEFGKIVKEAIREAGGVPFEFNTIGVDDGIAMGHIGMRYSLPSREIIADSVETVVSAHWFDGMVCIPNCDKITPGMLMAAMRINIPTIFVSGGPMAAGRTSDGRKISLSSVFEGVGAYQAGKINENELQELEQFGCPTCGSCSGMFTANSMNCLSEALGLALPGNGTILATSPERKEFVRKSAAQLMETIRKDIKPRDIVTVKAIDNAFALDMALGGSTNTVLHTLALANEAGVEYSLERINEVAERVPHLAKLAPASDVFIEDLHEAGGVSAALNELSKKEGALHLDALTVTGKTLGETIAGHEVKDYDVIHPLDQPFTEKGGLAVLFGNLAPDGAIIKTGGVQNGITRHEGPAVVFDSQDEALDGIINRKVKEGDVVIIRYEGPKGGPGMPEMLAPTSQIVGMGLGPKVALITDGRFSGASRGLSIGHVSPEAAEGGPLAFVENGDHIIVDIEKRILDVQVPEEEWEKRKANWKGFEPKVKTGYLARYSKLVTSANTGGIMKI

SEQ ID NO: 37 - DNA, 1677 bp, Bacillus subtilis
atggcagaattacgcagtaatatgatcacacaaggaatcgatagagctccgcaccgcagtttgcttcgtgcagcaggggtaaaagaagaggatttcggcaagccgtttattgcggtgtgtaattcatacattgatatcgttcccggtcatgttcacttgcaggagtttgggaaaatcgtaaaagaagcaatcagagaagcagggggcgttccgtttgaatttaataccattggggtagatgatggcatcgcaatggggcatatcggtatgagatattcgctgccaagccgtgaaattatcgcagactctgtggaaacggttgtatccgcacactggtttgacggaatggtctgtattccgaactgcgacaaaatcacaccgggaatgcttatggcggcaatgcgcatcaacattccgacgatttttgtcagcggcggaccgatggcggcaggaagaacaagttacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcaacatctccggaacgcaaagagtttgtgagaaaatcggctgcgcaattaatggaaacgattcgcaaagatatcaaaccgcgtgatattgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggctaagctggcgcctgcatcggatgtgtttattgaagatcttcacgaagcgggcggcgtttcagcggctctgaatgagctttcgaagaaagaaggagcgcttcatttagatgcgctgactgttacaggaaaaactcttggagaaaccattgccggacatgaagtaaaggattatgacgtcattcacccgctggatcaaccattcactgaaaagggaggccttgctgttttattcggtaatctagctccggacggcgctatcattaaaacaggcggcgtacagaatgggattacaagacacgaagggccggctgtcgtattcgattctcaggacgaggcgcttgacggcattatcaaccgaaaagtaaaagaaggcgacgttgtcatcatcagatacgaagggccaaaaggcggacctggcatgccggaaatgctggcgccaacatcccaaatcgttggaatgggactcgggccaaaagtggcattgattacggacggacgtttttccggagcctcccgtggcctctcaatcggccacgtatcacctgaggccgctgagggcgggccgcttgcctttgttgaaaacggagaccatattatcgttgatattgaaaaacgcatcttggatgtacaagtgccagaagaagagtgggaaaaacgaaaagcgaactggaaaggttttgaaccgaaagtgaaaaccggctacctggcacgttattctaaacttgtgacaagtgccaacaccggcggtattatgaaaatctag

SEQ ID NO: 38 - protein, 571 aa, Artificial sequence
  S. mutans DHAD variant I2V5
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIVLHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 39 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 40 - DNA, 1828 bp, Lactococcus lactis
tttaaataagtcaatatcgttgacttatttagaagaaagagttattctttaaatgtcaagttagttgactaaattaaatataaaatatggaggaatgtgatgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatagatatcaacggatgatgaaaagtaaaatagacaaagtccaataattttataaaaagtaaaaacattaggattttcctaatgttttt

SEQ ID NO: 41 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 42 - DNA, 1954 bp, Lactococcus lactis
ctagagttttctttagtcataattcactccttttattagtctattatacttgataattcaaataagtcaatatcgttgacttatttaaagaaaagcgttattctataaatgtcaagttgattgaccaatatataataaaatatggaggaatgcgatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataatttataaatagtaaaaaacattaggaaatacctaatgtttttttgttgactaaatcaatccctctttatatagaaaaccttagtttctcaaagacaacttaattaagcctgccaaattggaactcgcaaaatgtaatctatcctctgctccta

SEQ ID NO: 43 - protein, 550 aa, Salmonella typhimurium
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 44 - DNA, 1653 bp, Salmonella typhimurium
ttatcccccgttgcgggcttccagcgcccgggtcacggtacgcagtaattccggcagatcggcttttggcaacatcacttcaataaatgacagacgttgtgggcgcgccaaccgttcgaggacctctgccagttggatagcctgcgtcacccgccagcactccgcctgttgcgccgcgtttagcgccggtggtatctgcgtccagttccagctcgcgatgtcgttataccgctgggccgcgccgtgaatggcgcgctctacggtatagccgtcattgttgagcagcaggatgaccggcgcctgcccgtcgcgtaacatcgagcccatctcctgaatcgtgagctgcgccgcgccatcgccgataatcagaatcacccgccgatcgggacaggcggtttgcgcgccaaacgcggcgggcaaggaatagccgatagacccccacagcggctgtaacacaacttccgcgccgtcaggaagcgacagcgcggcagcgccaaaagctgctgtcccctggtcgacaaggataatatctccgggtttgagatactgctgtaaggtttgccagaagctttcctgggtcagttctcctttatcaatccgcactggctgtccggcggaacgcgtcggcggcggcgcaaaagcgcattccaggcacagttcgcgcagcgtagacaccgcctgcgccatcgggaggttgaaccaggtttcgccgatgcgcgacgcgtaaggctgaatctccagcgtgcgttccgccggtaattgttgggtaaatccggccgtaagggtatcgacaaaacgggtgccgacgcagataaccctatcggcgtcctctatggcctgacgcacttctttgctgctggcgccagcgctataggtgccaacgaagttcgggtgctgttcatcaaaaagccccttccccatcagtagtgtcgcatgagcgatgggcgtttccgccatccagcgctgcaacagtggtcgtaaaccaaaacgcccggcaagaaagtcggccaatagcgcaatgcgccgactgttcatcaggcactgacgggcgtgataacgaaaggccgtctccacgccgctttgcgcttcatgcacgggcaacgccagcgcctgcgtaggtgggatggccgtttttttcgccacatcggcgggcaacatgatgtatcctggcctgcgtgcggcaagcatttcacccaacacgcggtcaatctcgaaacaggcgttctgttcatctaatattgcgctggcagcggatatcgcctgactcatgcgataaaaatgacgaaaatcgccgtcaccgagggtatggtgcatcaattcgccacgctgctgcgcagcgctacagggcgcgccgacgatatgcaagaccgggacatattccgcgtaactgcccgcgataccgttaatagcgctaagttctcccacgccaaaggtggtgagtagcgctccagcgcccgacatgcgcgcatagccgtccgcggcataagcggcgttcagctcattggcgcatcccacccaacgcagggtcgggtggtcaatcacatggtcaagaaactgcaagttataatcgcccggtacgccaaaaagatggccaatgccgcatcctgccagtctgtccagcaaatagtcggccacggtataggggttttgcat

SEQ ID NO: 45 - protein, 554 aa, Clostridium acetobutylicum
MKSEYTIGRYLLDRLSELGIRHIFGVPGDYNLSFLDYIMEYKGIDWVGNCNELNAGYAADGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNN

SEQ ID NO: 46 - DNA, 1665 bp, Clostridium acetobutylicum
ttgaagagtgaatacacaattggaagatatttgttagaccgtttatcagagttgggtattcggcatatctttggtgtacctggagattacaatctatcctttttagactatataatggagtacaaagggatagattgggttggaaattgcaatgaattgaatgctgggtatgctgctgatggatatgcaagaataaatggaattggagccatacttacaacatttggtgttggagaattaagtgccattaacgcaattgctggggcatacgctgagcaagttccagttgttaaaattacaggtatccccacagcaaaagttagggacaatggattatatgtacaccacacattaggtgacggaaggtttgatcacttttttgaaatgtttagagaagtaacagttgctgaggcattactaagcgaagaaaatgcagcacaagaaattgatcgtgttcttatttcatgctggagacaaaaacgtcctgttcttataaatttaccgattgatgtatatgataaaccaattaacaaaccattaaagccattactcgattatactatttcaagtaacaaagaggctgcatgtgaatttgttacagaaatagtacctataataaatagggcaaaaaagcctgttattcttgcagattatggagtatatcgttaccaagttcaacatgtgcttaaaaacttggccgaaaaaaccggatttcctgtggctacactaagtatgggaaaaggtgttttcaatgaagcacaccctcaatttattggtgtttataatggtgatgtaagttctccttatttaaggcagcgagttgatgaagcagactgcattattagcgttggtgtaaaattgacggattcaaccacagggggattttctcatggattttctaaaaggaatgtaattcacattgatcctttttcaataaaggcaaaaggtaaaaaatatgcacctattacgatgaaagatgctttaacagaattaacaagtaaaattgagcatagaaactttgaggatttagatataaagccttacaaatcagataatcaaaagtattttgcaaaagagaagccaattacacaaaaacgtttttttgagcgtattgctcactttataaaagaaaaagatgtattattagcagaacagggtacatgcttttttggtgcgtcaaccatacaactacccaaagatgcaacttttattggtcaacctttatggggatctattggatacacacttcctgctttattaggttcacaattagctgatcaaaaaaggcgtaatattcttttaattggggatggtgcatttcaaatgacagcacaagaaatttcaacaatgcttcgtttacaaatcaaacctattatttttttaattaataacgatggttatacaattgaacgtgctattcatggtagagaacaagtatataacaatattcaaatgtggcgatatcataatgttccaaaggttttaggtcctaaagaatgcagcttaacctttaaagtacaaagtgaaactgaacttgaaaaggctcttttagtggcagataaggattgtgaacatttgatttttatagaagttgttatggatcgttatgataaacccgagcctttagaacgtctttcgaaacgttttgcaaatcaaaataattag

SEQ ID NO: 47 - DNA, 1641 bp, Macrococcus caseolyticus
atgaaacaacgtatcgggcaatacttgatcgatgccctacacgttaatggtgtcgataagatctttggagtcccaggtgatttcactttagcctttttggacgatatcataagacatgacaacgtggaatgggtgggaaatactaatgagttgaacgccgcttacgccgctgatggttacgctagagttaatggattagccgctgtatctaccacttttggggttggcgagttatctgctgtgaatggtattgctggaagttacgcagagcgtgttcctgtaatcaaaatctcaggcggtccttcatcagttgctcaacaagagggtagatatgtccaccattcattgggtgaaggaatctttgattcatattcaaagatgtacgctcacataaccgcaacaactacaatcttatccgttgacaacgcagtcgacgaaattgatagagttattcattgtgctttgaaggaaaagaggccagtgcatattcatttgcctattgacgtagccttaactgagattgaaatccctcatgcaccaaaagtttacacacacgaatcccagaacgtcgatgcttacattcaagctgttgagaaaaagttaatgtctgcaaaacaaccagtaatcatagcaggtcatgaaatcaattcattcaagttgcacgaacaactggaacagtttgtcaatcagacaaacatccctgttgcacaactttccttgggtaagtctgctttcaatgaagagaatgaacattaccttggtatctacgatggcaaaatcgcaaaggaaaatgtgagagagtacgtcgacaatgctgatgtcatattgaacataggtgccaaactgactgattctgctacagctggattttcctacaagttcgatacaaacaacataatctacattaaccataatgacttcaaagctgaagatgtgatttctgataatgtttcactgattgatcttgtgaatggcctgaattctattgactatagaaatgaaacacactacccatcttatcaaagatctgatatgaaatacgaattgaatgacgcaccacttacacaatctaactatttcaaaatgatgaacgcttttctagaaaaagatgacatcctactagctgaacaaggtacatcctttttcggcgcatatgacttatccctatacaagggaaatcagtttatcggtcagcctttatgggggtcaatagggtatacttttccatctttactaggaagtcaactagcagacatgcataggagaaacattttgcttataggcgatggtagtttacaacttactgttcaagccctaagtacaatgattagaaaggatatcaaaccaatcattttcgttatcaataacgacggttacaccgtcgaaagacttatccacggcatggaagagccatacaatgatatccaaatgtggaactacaagcaattgccagaagtatttggtggaaaagatactgtaaaagttcatgatgctaaaacctccaacgaactgaaaactgtaatggattctgttaaagcagacaaagatcacatgcatttcattgaagtgcatatggcagtagaggacgccccaaagaagttgattgatatagctaaagcctttagtgatgctaacaagtaa

SEQ ID NO: 48 - DNA, 1647 bp, Listeria grayi
atgtacaccgtcggccaatacttagtagaccgcttagaagagatcggcatcgataaggtttttggtgtcccgggtgactacaacctgacctttttggactacatccagaaccacgaaggtctgagctggcaaggtaatacgaatgaactgaatgccgcgtacgcagctgatggctatgctcgtgaacgcggtgttagcgctttggtcacgaccttcggcgttggtgagctgtccgcaatcaatggcaccgcaggtagcttcgcggagcaagttccggtgattcatatcgtgggcagcccgaccatgaatgttcagagcaacaagaaactggttcatcacagcctgggtatgggcaactttcacaacttcagcgagatggcgaaagaagtcaccgccgcaaccacgatgctgacggaagagaatgcggcgtcggagattgatcgtgttctggaaaccgccctgctggagaaacgcccagtgtacatcaatctgccgatcgacattgctcacaaggcgatcgtcaagccggcgaaagccctgcaaaccgagaagagctctggcgagcgtgaggcacaactggcggagatcattctgagccatctggagaaggctgcacagccgattgtgattgcgggtcacgagatcgcgcgcttccagatccgtgagcgtttcgagaattggattaatcaaacgaaactgccggtgaccaatctggcctacggcaagggtagcttcaacgaagaaaacgagcatttcattggtacctattatcctgcatttagcgataagaacgtgctggactacgtggataactccgactttgtcctgcactttggtggtaaaatcattgataacagcacctccagcttctcccaaggcttcaaaaccgagaacaccctgactgcggcgaacgatatcattatgctgccggacggtagcacgtattctggtattagcctgaatggcctgctggccgagctggaaaaactgaatttcacgtttgccgacaccgcagcaaagcaggcggagttggcggtgtttgagccgcaggctgaaaccccgttgaaacaggaccgttttcaccaggcggtgatgaattttctgcaagctgacgatgtcctggttacggaacagggcacctcttcttttggcttgatgctggcgcctctgaaaaagggtatgaacttgatctcgcaaacgctgtggggtagcattggttacacgttgccggcgatgattggtagccaaattgcggcaccggagcgtcgtcatatcctgagcattggtgatggtagctttcagctgactgcgcaggaaatgagcaccattttccgtgagaaactgaccccagtcatcttcatcattaacaatgatggctataccgttgagcgtgcgatccatggcgaagatgaaagctataacgacattccgacgtggaacttgcaactggtggcggaaaccttcggtggtgacgccgaaaccgtcgacactcacaatgtgttcacggagactgatttcgccaacaccctggcggcaattgacgcgacgccgcagaaagcacacgttgtggaagttcacatggaacaaatggatatgccggagagcctgcgccagatcggtctggcactgtccaagcagaatagctaa

SEQ ID NO: 49 - protein, 312 aa, Saccharomyces cerevisiae
MPATLKNSSATLKLNTGASIPVLGFGTWRSVDNNGYHSVIAALKAGYRHIDAAAIYLNEEEVGRAIKDSGVPREEIFITTKLWGTEQRDPEAALNKSLKRLGLDYVDLYLMHWPVPLKTDRVTDGNVLCIPTLEDGTVDIDTKEWNFIKTWELMQELPKTGKTKAVGVSNFSINNIKELLESPNNKVVPATNQIEIHPLLPQDELIAFCKEKGIVVEAYSPFGSANAPLLKEQAIIDMAKKHGVEPAQLIISWSIQRGYVVLAKSVNPERIVSNFKIFTLPEDDFKTISNLSKVHGTKRVVDMKWGSFPIFQ

SEQ ID NO: 50 - DNA, 939 bp, Saccharomyces cerevisiae
atgcctgctacgttaaagaattcttctgctacattaaaactaaatactggtgcctccattccagtgttgggtttcggcacttggcgttccgttgacaataacggttaccattctgtaattgcagctttgaaagctggatacagacacattgatgctgcggctatctatttgaatgaagaagaagttggcagggctattaaagattccggagtccctcgtgaggaaatttttattactactaagctttggggtacggaacaacgtgatccggaagctgctctaaacaagtctttgaaaagactaggcttggattatgttgacctatatctgatgcattggccagtgcctttgaaaaccgacagagttactgatggtaacgttctgtgcattccaacattagaagatggcactgttgacatcgatactaaggaatggaattttatcaagacgtgggagttgatgcaagagttgccaaagacgggcaaaactaaagccgttggtgtctctaatttttctattaacaacattaaagaattattagaatctccaaataacaaggtggtaccagctactaatcaaattgaaattcatccattgctaccacaagacgaattgattgccttttgtaaggaaaagggtattgttgttgaagcctactcaccatttgggagtgctaatgctcctttactaaaagagcaagcaattattgatatggctaaaaagcacggcgttgagccagcacagcttattatcagttggagtattcaaagaggctacgttgttctggccaaatcggttaatcctgaaagaattgtatccaattttaagattttcactctgcctgaggatgatttcaagactattagtaacctatccaaagtgcatggtacaaagagagtcgttgatatgaagtggggatccttcccaattttccaatga

SEQ ID NO: 51 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 52 - DNA, 1083 bp, Saccharomyces cerevisiae
ctagtctgaaaattctttgtcgtagccgactaaggtaaatctatatctaacgtcacccttttccatcctttcgaaggcttcatggacgccggcttcaccaacaggtaatgtttccacccaaattttgatatctttttcagagactaatttcaagagttggttcaattctttgatggaacctaaagcactgtaagaaatggagacagcctttaagccatatggctttagcgataacatttcgtgttgttctggtatagagattgagacaattctaccaccaaccttcatagcctttggcataatgttgaagtcaatgtcggtaagggaggaagcacagactacaatcaggtcgaaggtgtcaaagtacttttcaccccaatcaccttcttctaatgtagcaatgtagtgatcggcgcccatcttcattgcatcttctctttttctcgaagaacgagaaataacatacgtctctgcccccatggctttggaaatcaatgtacccatactgccgataccaccaagaccaactataccaacttttttacctggaccgcaaccgttacgaaccaatggagagtacacagtcaaaccaccacataatagtggagcagccaaatgtgatggaatattctctgggataggcaccacaaaatgttcatgaactctgacgtagtttgcatagccaccctgcgacacatagccgtcttcataaggctgactgtatgtggtaacaaacttggtgcagtatggttcattatcattcttacaacggtcacattccaagcatgaaaagacttgagcacctacaccaacacgttgaccgactttcaacccactgtttgacttgggccctagcttgacaactttaccaacgatttcatgaccaacgactagcggcatcttcatattgccccaatgaccagctgcacaatgaatatcactaccgcagacaccacatgcttcgatcttaatgtcaatgtcatgatcgtaaaatggttttgggtcatactttgtcttctttgggtttttccaatcttcgtgtgattgaatagcgataccttcaaatttctcaggataagacat

SEQ ID NO: 53 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 54 - protein, 387 aa, Escherichia coli; identical to SEQ ID NO: 53

SEQ ID NO: 55 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 56 - DNA, 1170 bp, Clostridium acetobutylicum
ttaataagattttttaaatatctcaagaacatcctctgcatttattggtcttaaacttcctattgttcctccagaatttctaacagcttgctttgccattagttctagtttatcttttcctattccaacttctctaagctttgaaggaatacccaatgaattaaagtattctctcgtatttttaatagcctctcgtgctatttcatagttatctttgttcttgtctattccccaaacatttattccataagaaacaaatttatgaagtgtatcgtcatttagaatatattccatccaattaggtgttaaaattgcaagtcctacaccatgtgttatatcataatatgcacttaactcgtgttccataggatgacaactccattttctatccttaccaagtgataatagaccatttatagctaaacttgaagcccacatcaaattagctctagcctcgtaatcatcagtcttctccattgctatttttccatactttatacatgttcttaagattgcttctgctataccgtcctgcacataagcaccttcaacaccactaaagtaagattcaaaggtgtgactcataatgtcagctgttcccgctgctgtttgatttttaggtactgtaaaagtatatgtaggatctaacactgaaaatttaggtctcatatcatcatgtcctactccaagcttttcattagtctccatatttgaaattactgcaatttgatccatttcagaccctgttgctgaaagagtaagtatacttgcaattggaagaactttagttattttagatggatctttaaccatgtcccatgtatcgccatcataataaactccagctgcaattaccttagaacagtctattgcacttcctccccctattgctaatactaaatccacattattttctctacatatttctatgcctttttttactgttgttatcctaggatttggctctactcctgaaagttcatagaaagctatattgttttcttttaatatagctgttgctctatcatatataccgttcctttttatacttcctccgccataaactataagcactcttgagccatatttcttaatttcttctccaattacgtctatttttccttttccaaaaaaaactttagttggtattgaataatcaaaacttagcat

SEQ ID NO: 57 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 58 - DNA, 1173 bp, Clostridium acetobutylicum
gtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 59 - protein, 139 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMI

SEQ ID NO: 60 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 61 - protein, 533 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVI

SEQ ID NO: 62 - DNA, 1000 bp, Artificial sequence
  HXK2
ccattccacaactttcatttagcactactgggacaagcagtagtgaaagcgttgatagcactacggcacaaaccccaactgatccagagagttactggtcggataaccggtgcaaacatagtgattgtcaagagcttagtccattcgcatctgtatttgatctcattgaccactatgaccacacgcacgccttcattcctgagacgctggtaaagtacagctacattcatttatataagcctagcgtctgggatttattcgaatactgaacgccatagaagagcaatttccgtcctctactcatgtgattcgaactatgaaacaaagaaaaggcaagagtaaatagcgtgatacctattacgtattacgtatacatcctattattcttgaaaaaaagtgcggggctccagagctccacattggtgaccccagagtatactgctctttctaatgccttttccatcatgttactacgagttttctgaacctcctcgcacattggtacctagaaatggctatcatgccggacggcaccgggcaataaaccggacggcacaaaaaaatcgaagaaaagagatttctttttctcgcgggcagtttttccggtcgatcgacattcgtacggtactttctctgtttcagggacatcatgttgtaaaagaaaaagacagtttaggtaatcgttcttttctttctgaaaaattttccacgacgacgacgacgaccacgaaacacctttgattgcgagatccacgaaattacctcctgctgaggcgagcttgcaaatatcgtgtccaattccgtgatgtctctttgttgcaccttcgccactgtcttatctacaaaactataaaaaagagtaatcctaccccatatctaaaaaaaattccttaacttttataacttaacttcaaagtttcttaatattttttcgctttttctttgaaaaggttgtaggaatataattctccacacataataagtacgctaattaaataaa

SEQ ID NO: 63 - DNA, 1000 bp, Artificial sequence
  IMA1
ggaacggggctgtatgtttatgattgctcgaatcacgtttttcttgttttttcgtcaagaattccagtcaagttttccaccaccttgacccttaaagcatcgacttttgtgctcttgaatgtgtttctaagaatacttgtaaaggacaccctctaatttcgtgtgcacttttcacatattatcaagacaatcgttcctgtactcagatgcactgttactgtaaagactactatacaacaagcgaaaaatgatgttcgaaaacctttatttctattttgaaaggcatgtgtctcgaggtccttgctttattgtgggtggtcatgccattctgtaaaccttacggtactgctccgtctatatctttgaggttgttatttccccacaaatatgcgtttctaaccgaatattcattcagtcggaccggacaatagctcttaactgcgtttaccggagtaaatatcgtaagaatttgcatgcggtgaaatacagggaaaataagaaattacaccctaatacaaaaagaaaactaagtttcacaatacgtaaggatattttagtggggagaatatttcggagaataaagtttccaactccgcggtgtgaacaaccgctcagcacgcagcgttattctcgagaaaagtggccctgaaataaggaaataaagttactaatgttttttcgctgtacgatatcaaatgtgacgaagtaggcaccccacgctataaattggctactaaagtttatgtcagtacttgggatcgttgaaatactcggataaattatgttccttatttttcatggttttcgtcataccacagtttaccccagaatgagaaaggatctccttttgaaataaaaagtacttaagggcaatgatattgagttgctagacgtttggttagacgcctgttttgaaataaaaaagctgtctcaaattaatcgagcaagcacagatcaaacaagatacaaacaaagcttttcaacgtaatatttactatcg

SEQ ID NO: 64 - DNA, 1000 bp, Artificial sequence
  SLT2
gtggtgaaaatgaaggaaatttacaagattgtggatgacgaagttgtcatggacatgagattagtgagtcgggtcattggtaatcccttgttaaaggaatcaaaggagtttcgtcaagatttgaatgccaggccattagctagattggaacgtttgaaaatcttgataaactatgcagttaagatctctccgcataaggaaaaattcccctatgtgaggtggacagtgggtaaaaacaagtacatacatgagctcatggtcccagagcgctttcccattgatattcccagagaaaatgtcgggttagaaagaactcagattccattaatgctatgctgggcactgtccattcataaggcacagggtcaaactattcaaagactaaaggtcgacttgaggagaattttcgaagccggccaagtttatgttgcactgtcaagagcggtaactatggacaccttacaggtcctaaactttgatccaggaaagattcgcaccaatgaaagagtaaaagatttctataaacgtttagaaactttgaaatgacttgcaacgaataaatgcatatactctagttgaagttttcttttcttgttctatacaggttcgaatacttgtgagcctatctgtataatttaacagaatcccgaaatattcatctagaagccatctatttagctaagcctacgtatgcggcgatttttatattatctttttttttttttatagaagactgcgaaatgttggcagaatggaaagtttcagtgttaaaaatagaaactgaaaaaggagatctagccaggaatatatcgaaaaaaaaagtgagggaaatcagatcctacacaaatatttagatttaattgaagaccctggtctgccagatatatatatatattagacgaactgtgcattcagtcagcaaatctaggccacagattttcttattgaagctatcaaaatagtagaaataattgaagggcgtgtataacaattctgggag

SEQ ID NO: 65 - DNA, 1000 bp, Artificial sequence
  YHR210c
cataaaactgtaagaattgtaaagcaaaactggtgcactgctacatcaactcttatcttcttgcatataaatttaaatgaaaaggaactactttgtaattatcgatattacattgaaacctatgaggaagattatgacatgataacactaaactattgctggccagcacccgaaccgagtgtccaaattgagttttgaatcagttttcagctcagaacagattccccttttcctttattaggtcgtatcatcattctatgtgcaactactgcagcatttgcgtataattatggaaataaaattatacgaaagttgatacttggttgtaaatggcctaagaatcggaaaaaatggaattctattggcatgctatcttgtttaatgagattatgaagttgaatatcatttctgcgttttgcattcaaacaatatcatttcaaaggcctatcgctgtgagcaatacgctggatttgctcatcgcacagtaagcctgcactaccccaccaaattacaagaatatagtagagatcagaagcttatgagaagtcacttccaaaattcgcgttgtccaacatccttggaataatttttcctccttatgcatccaggtgattgacactttttgacgggataaatgcaggtggaggtgggggaggggtaaagtttcgtaaagagtgtcaggtcgcatatctttgcactgcgcagaaattctcgcagaaaattacgctacggagaaattctcgggcactaatatcgtacaaaaattaggtttgttacgtgataatatggccttgtacctgttaagtttttccgacaattagttactagctgaatatcttaaaaaaaggtaaaagtaactcctccatggtttggttattcatgcgtaaacaccttggatgctctgagatataaaatcagaatgtactacgaagtactggaactcaattgtacattatgttactgcaataagtaaattcacctaaaaagccaaaaagtgata

SEQ ID NO: 66 - DNA, 1000 bp, Artificial sequence
  YJL171c
atgaaaacaaggaatatacggggaacatataaagggtcagcgtcgttgactagtctgtcatttatattgtgcttgtacataggactacaaaaattcaaagaatgacgacaagaaacatgactactttagcatcaagtattgagcataaaacaaaacatctagcgcgcccttcgagaacgatgagaacccttggatgaaaaaatactgttgccaatgcaaatcatgtaaaatgagtgtgcctgtccaaccttggctgccaaggttttttgtttttggaatcttgtgccccgttttctggttggtgaatcttttagcctggtggttcttacagtattggcaaccacatgaattggaattccacgacttgcaagaagacgaatacccaggattttatgaatatgaagccataactaaaagaacggtgatacccataaaggaagaagtcctccaagagattcgcgtcatgcagaatttcagtgatagcaacagcgaagaatattacgagagcaaagacggtatgccgtcgtctttcctaaacgtgaacacggaacaagtcgaggacgagaatgacaccttgaaaaaatatcgttacgcgtttttgaagaaggtagcccacgatgtgctggagtcgcacgatttattacgtaagaccttccgcgactggaatttacggtctttgcttggcctcctcatcgattctatactcataatatttgtagttctcctatgtaagaaaagccgttaggtagatctaaagacagaaaatgatatcagcctctttcttttccacagcctccatttcccatttttgctgtatctgcaagggttcctttttccatttttttttttcaaagtcttggctccgcccttttcttttgtctattttgccgagcttaggttatttataaacctgaaaatcctcgagaaacaatccttatcgaaggaagcagccataatagaacatactcaagtgcaaacattacaagcttaaataagacaa

SEQ ID NO: 67 - DNA, 1000 bp, Artificial sequence
  PUN1
gcccctctgtcgctgtagaatcttagatttgccatggtaaagacctacggactgatcgataaagatatctttttcaccttctcgaagaagaggttggcccgatgacgtagtttccacataatgccagtactccatcttttgaaaaaaaaagaatgactttcaaaacacacttcctgattcctagccgaggtcccctattctatcttggatgaccttttctctgcttttttgtttttcagtctccatcttttccagatgaaacatgtgaaaccctttttcctcttcgttgatcctaaaaaaagcgcgtctggaaaacaaggcgaagcttcctcacatggaaaaggcttcttaatatgtcgctacgcctcattcccattgcatacagaaagcaaagtagtgcgttttgcgaacgtcacagatcaaaagtttggccatgcattgtcattctttttttcttacatacactgcaccagacatgtcggttccgccccgctaacggttagtcagccacacattgacgtacactgtgaacagcctatttctttccatgtatctcagtgcccagcttatgagaactgtcacagcctcccacttgaccctcagagccctctccactccccccctctttcaacatcgccagatagccgccgttgaatggtgcgggacaacccggcctggcctggccaggcaaaaaaggacgcagcacgcctcgagcgttatttccaaatcgggcgtactatcagccaagcccagctcggtatttttagcgcttctcgcaggaaaattggctgagaagtatatatacgcgagaatgttgctcttccatgtctcagtagtcaatgagtgtccagtggtgtttcattctggaccagttgtttggaagtagaactaaaagaaactagatcaagatcatacaacgctgcgcagtagtgaaacttgattaaagcaatagagaactattaagaaaaaaacaaacacatcatcgaaggacgctataagc

SEQ ID NO: 68 - DNA, 1000 bp, Artificial sequence
  PRE8
tatttttggatagtttcaataggaacatcacgtattatgaaagtccggaaaatacattacaattcgccaatattttatccagccaaacaagcttcataaatttactatcgacgtacaacttaattgcccattcagatcacctcatgaacttcaacgtcggtggaatggcagcaaaagttaaaaaagaaatcctgaatcaaatgctaaacaatttgtatgattccatcagactattgtctcccagcattgaaaatgacaaatcaatgaaggaaaaattgagagaaaaagtaaagaactactgcagattcaaagcttatttgaagtcacctgaattggacatggatgaactgaagaccttggtttcagtcgagtcttttttaaatcctttcactccttcaatgttgttcaataacttgatcgaaactatttatataaacgaacacgcttcctcgttagtccttcagaatgggttgatctattcactccaacagaaaggtctcaataagatactgagctatctagaagaaagcttcatcactagtggaaatgatgccaatattgaaaaggtcagggaattcaggtcccttttaaggaagagcaagcctcttcaagcatgacctttacaatttgatattttatttatttcactgttataacataattacaagtaattaatgtcatataataataataataataataataataataataataataataataataataataataataataataataataataataataataataataataataataataataatagtaataataacgtctatataagaagtatctacaccattgcttacgcttttattttttcctttcgacgtgaaaagcgataatagcttattaaggtggcaaaattgtccccggccccaataagctgagagtggaataggtgaagcattttatatttttagtttcaattagtagtaagcaaccataagacaccaatcaacacagttctataatt

SEQ ID NO: 69 - DNA, 1000 bp, Artificial sequence
  COS3
aatttatatacacttatgccaatattacaaaaaaatcaccactaaaatcacctaaacataaaaatattctatccttcaaccataatacataaacacacttaattgcgtcttaatactatcatggtatcattaacttaaagttccttaatatcgtcataccactatgctctattccatatattgtaatataactgtactctatagtcatacagacgcttttacttcaccccatcttatactattgtcatagaatctcacactgacgcatgattaaaacgaataatttttactgtaagggctgccatccgcgctctatccttttgtttgcaatatttatatacagaatctcaaaacaagcgggagaagtgctaattacccagaggtcatgcatgatctgagtaccaccgtacctctaggttttgctttgatccgttttacagtgacaccgaacataaggggaagctattgacatggtatcgaaaggttgtccacattgggaagtaacttggttctatgaatcttcatgtcagatacgtaggacagactctttcctgtgtaaatatttgtgacagctacgtctattttctactagatgtttacacagttttgtcacaggaaatctacgcttaaaatatgtatttcattcaagcggtaaccgctgtacgagcagtgacattgctggtcgcaccctaaatgtgaaccaacgttacggcacaccgtgatgtacccgcattaaagttttgtaaattcgttattacgattatcgagttggctagatagaaaaccggaaatgtaatggatgcccttttcgaatagctgagtttctttgcctaaaatagcccaatattgttgccctttttctatcacgaggttactgagccattgcatgaacgcgcgcgcctcggcggctttttttttctgctgtgctgtataaaagcgaaaagccagaagttactatctcgaataaaaaacccctcgaactgccatctcactaccgaaa

SEQ ID NO: 70 - DNA, 1000 bp, Artificial sequence
  DIA1
actacgttaaaattgagcagccatgattaccgttttacgatgtaacgcttttttaaccttaatagatagattccctcatatatttataactatgtacccacataagtatacttttggaatgataatactaacgagataaaaaaccgttgaaaaatttctaagttttcttgaactaaaaatagccaaaattctccatccacttgttgttgcaaaatgttacgaaggcgaggttcttggaaatctggatgattatgggaaaattcgttcaacaagaacgccgagcctggacgaaatacttagtcggcatggaactagttatgaatgacttttcctatataagatctacaaccgtttccaattcaccatgagatatatatatgtttaacgaatcaggtactcgtccgaagcatttcgagtaatgcaaccccacaagtgtcccccaagaattcactgggatttttgatgaccgaaagaaagctattgcagctgctacggcgtcctttcatctccctttctttattcacggccttaagagcttgtccgcttcgccccaaaagcctaattgcttagagcatgagagttgaaaacgaccagaagtgtcgttttaggtaagcattcggccataatgaacagtaagtttgtccgaaaaatacaccgctagggttcgataacgaagagcgcatatcaattgcgttgtcgacttaattgctctcttagtacaacggtatgtgtcagtgataaaatcctaccggttccactttttgtgagcgatagatgacggaacgtatgcgcctatttcttttttttggcgcagcaaggacaatggtccctttttgagaaaatgttgtaggcttggccatcggcgtgaacgcatataaaaagaagactgccaacatgacagtatttgaaacaactacagatttggcaaagttcgccaagataaagaagaccgaagcaggtgctaagcttccattaaaagtttttcaagaaaaatctgatt

SEQ ID NO: 71 - DNA, 1000 bp, Artificial sequence
  YNR062C
taaaactgagctattctatatatgtatgcttgagtgtagagctcgaattatataaaaaaaaaaatacattactgtgatccgccaggaaaaggcagtaaaaaaagctgatacatatttacaacggcgaatcgcgaagacacttacttttctcacatgcctttgtctttatgcttacaaaggaccactttacttattcttctagtctttcgcttcgacgtcggaatctcaaaaaaatatgggacagctgtctggaaatgggggacagtctaggtaattagaattaccaaacgcctactgttctcttctattactaaaataagttgattttactattttgatacagaagtatagcgcccaaacaagcctatcccattctaagcggaacaaaatttcctaatctggaaaagaatcgccgtcaataaataatcatcatataaaagatcagttaacattactttctagttgtcaagtactctttcgttcttcttaattgtatctcaattgcattacaaatccatccatttcactcgacggtggtacaataaattaaaatgaaaaaaatcttaggtgtcaagaaacacatgtttttgaaatacttggctgtaattttgatagctgtccaattcagtttcgactcctgtgtatatctctcaagtgtggttgactacatcaaagaagtaagtgttaatgtaaatctaagataatttcaatgaactagtatgctaacaatttagagaacggagaaaccaatccggacaaattcttgttcagaattcaagccatttctgcagcagttcaagtttttgtttcattcattattggggatattgctgcttttcttgggtccattaaatgggtaatagtggggctttactttttatcctttgtgggtaattttttgtattcatgcggcggtgcagtgtcgctaaacacccttctcggtgggcgtattatatgtggtgcagcatctgcttctggtgctatagtgttcagttacatc

SEQ ID NO: 72 - DNA, 1000 bp, Artificial sequence
  PRE10
aaaacattgacagctcagacgatagaaaaacatgtgatacgaatcatttcgccgttgtgtgttttaagaagaaaaatgttaatgttagcaagacgaaatcacacgtataaataaatatagcagcacgaccagcttggaagttcaaaaaaaaaaaaaaagtgaagatagacaatcataggcacaatttctgatggtagtgaacatataatgggcagaccaagctctagcaacttaatgcagagaaagatttgaagctaccgccgcgtgttcagcgtgactctctataataggccatttatgtacaagatcttctgcaatcctatcaagtattcgtactactattttccctgccaattagacgtgatgaaaaatattgctgggctttggtttgaaaacgactatttaaataaatattacctttccttttcttcaattgtttctcatctccttcggcctccgttaagtatatgtttttttacgcgttgtcaatgacaataccgcaggatgcgaatgtgtgacttcgtaatcatcggtgtgaaaaatactctaactttaaacaaaggtaacatctaaactgtctggaaatatgcagctattcccatgtatacacactagaaagaacagtatttcccaggtcccagaagttctctgtatatgtagtcctacccgtaatttaccctttgaagagataatctgagcactcagatttcgaccctgatggggtcacttttctgaatcattgctgacattgcttctgaaaagtgttgcctgtgtctttattccacttatgagttggtaagcttcctcacaatttataatgggttccgatagctaaatcacgtttaacgaagatttcgccgcccctctaaatcggtggcaaaaataagaaagtaagtggtaatagtggcggtagagggtttatttggtagtgcagttgtaaagctttcaaacacattcgagcgtcgcatcatcaaattaacaaaagcataactcttcagca

SEQ ID NO: 73 - DNA, 1000 bp, Artificial sequence
  AIM45
gggaaacatatcagtatcgtgaatccatcctcaaaaatatatcattcatcccataaacagattgttaaaacgcctatccctaagagtggcctttctccaattgagagatgccctttcaatggtcaaaatattaaatgctactcaccaagaccactagatcatgaaagtccccaacgtgatttcaataataactttcagctgagaatactgaagagctcggtgttgcaaaggagacaatcaacacagaatagttgaaaaattcgttggtactagcttcgggtcggttagctgcgctgctatgcatttgttaatatctccatcgaatttttgttgtttcgttcaataactttattacttcccccttggactctttcgttctatcgttctacaagtccagccaaaatttttcccctcttccttttcttttgttcacttcttagctcacttatataattatatactgatatttggattcttttgttgcaaatatgctctcccagatttttctgttgagatgattcatgctttacatggattgagcattagagagtaactatatccaatttcgtaagacgagtatctactttcccttgtccccagtaacctcaggaacgtgacaactacttttcttaaactgtcaacagccaatgataccgtattcaatgcatgtcttgggattaagccactttgattgagttccgtattagtagtgagaattaaatcttgcaagatataagaattactcaacagagacgagatactttcctttttttggttcttttgttgttacttggcttaatggacaaagtctgctcgcatattgtatatgttactcttacgatgtgactccgcccgtttattatgacttttcggtacatttttagggcctcgaacgaaaatctactaactaaaaattaaaaacaattaaaataatcaacaaacaaaatctagtgatataatttactaccattaacggtaaagcagctaattgttaatttct

SEQ ID NO: 74 - DNA, 1000 bp, Artificial sequence
  ZRT1
agacttgagatagatgtacctggagagaaaatctataataaaaaaaaaaatactttcaggtcagatatttctttttttgtccctaataaaaagaaatggtattctgccaaacaccaaagtgccaaataagcattattttacatagtacgaaatggaaattacgtcaattatcgacattatgacataaaattggatttaacaagatctctgaatctgatatgcttctttcattagggtggaaataacagcatttgagagaagcaattgccaagcttctatgaaaattttctagaaggcaagagtatttcagactttcctaatatgaaaggacaaattgacactaatgtctgattatggccaattcctgcggtaaattacacggcgattacggcgacatgagctcacattcatcactctatgggacaaatgtttccaaactgggcgcaacaaacacctgatgtgactcctaccctttggacaatgcagatccacgctacggcaaattagtcaaatgcactagaacatggcgcaagtacttattgtgacctttggggtaccgttaccgtcagttttcttcagctaaggcgcgcgcgccagataactaaaaaaaaatatagttgctgcttaaaaaacaatacacccgtactctcttgcctgtaaaaacctcgaaggaccaaagataccctcaaggttctcatctgtgcggtattcttcaaattacaatgacatttcccaaaattatcagatgtgctcaggtatcttctctccaatgagatgagacagatgaacatatttgaccttgaaggtcatggaaagtaggttgagagcaaatgtgtagaacgaaattaagaaaaaaagaaattacgcacggcattagctcgatgacttagttataaatagaggcctggtatcggctgtcatgatctcatctcttccctatttacaaaaaaactgcaagtatagacaataaaacaacagcacaaatatcaaaaaaggaatt

SEQ ID NO: 75 - DNA, 1000 bp, Artificial sequence
  ZRT2
tccgggtaccattgaccagtacgtcaaggaactacccgacaaactattcgagtgcttataccctaactgtaacaaagtattcaagcgtagatacaacataaggtcgcatattcagacacatttgcaagatagaccgtattcatgcgactttcccggttgcaccaaggcgtttgttcgcaatcatgatttaataagacacaaaatctcccataatgccaagaaatacatctgcccatgcggaaagagatttaatagggaggatgctctaatggtgcatagaagtcggatgatttgcaccggcggtaagaaattagaacattcgatcaacaagaaacttacatctcccaaaaaaagcctgcttgacagcccgcatgacacaagtcccgtaaaagaaactatcgcccgggataaagatgggagcgtcctaatgaaaatggaggaacagctgcgagatgatatgcgcaaacatggattactggatccacccccatccacagcagcgcacgagcaaaactcgaaccgcaccctttcaaacgaaactgatgctctctgacgaacatttatctatgcatgatattaacataataaataatagtaacaataatataatacatttatttctttacgatttacgtacactgtagtcttaagggccaaaaaaggcaatagtcatcgtttatagggacataaccctaaaggttatatgcgactccttctcgaaaatgaaaatttttcacaacctttagggtacattgccctttaactactacaaaccagcttcacacatcctttggtaaaacacagtgtgacgattttaaatgacgcaaccttttgggagacacgatctaaaacccactatatatgatgatactgtttattgaatcatacaccctgttggttagaccggaagaaagctaaagttgatctccgaaaatacaacggtgcatcaaccaaagaaaacattgcaggcgtttccaagtgacactgcatattcgaaagt

SEQ ID NO: 76 - DNA, 1000 bp, Artificial sequence
  PHO84
tcacttcgtttttttaccgtttagtagacagaatgcgagagtgataaagaagaggcggttaatcaatgaaaaaaaaaaaaaaaaatttaaaaaagaaaagagaaaaggaataaaaaagtgtcacgtgataaaaatcactacccggagatgacttcaaacgactcggtatactctgcctaataaaccttaattttcttacaaaaaaaaaagattcaataaaaaaagaaatgagatcaaaaaaaaaaaaaattaaaaaaaaaaagaaactaatttatcagccgctcgtttatcaaccgttattaccaaattatgaataaaaaaaccatattattatgaaaagacacaaccggaaggggagatcacagaccttgaccaagaaaacatgccaagaaatgacagcaatcagtattacgcacgttggtgctgttataggcgccctatacgtgcagcatttgctcgtaagggccctttcaactcatctagcggctatgaagaaaatgttgcccggctgaaaaacacccgttcctctcactgccgcaccgcccgatgccaatttaatagttccacgtggacgtgttatttccagcacgtggggcggaaattagcgacggcaattgattatggttcgccgcagtccatcgaaatcagtgagatcggtgcagttatgcaccaaatgtcgtgtgaaaggctttccttatccctcttctcccgttttgcctgcttattagctagattaaaaacgtgcgtattactcattaattaaccgacctcatctatgagctaattattattcctttttggcagcatgatgcaaccacattgcacaccggtaatgccaacttagatccacttactattgtggctcgtatacgtatatatataagctcatcctcatctcttgtataaagtaaagttctaagttcacttctaaattttatctttcctcatctcgtagatcaccagggcacacaacaaacaaaactccacgaatacaatccaa

SEQ ID NO: 77 - DNA, 1000 bp, Artificial sequence
  PCL1
tcagtgacgacgttatctatgaatgctgtggggcacccagaccaagtgacttaaaggcagtattgaagtcgatactggaggacgattggggtaccgcccactacacacttaataaggtacgcagtgccaagggtctcgcgttgatcgacctaatcgagggcatagtgaagatactggaagactacgaacttcaaaatgaggaaacaagagtgcatttgcttaccaaactggccgatatagagtactcgatatccaagggtggcaacgaccagattcagggcagcgcggtcattggcgccatcaaggccagcttcgagaacgaaactgttaaagccaacgtataatcgacgcaaatatgtatagatacaatatgtacagaacaactgcattgtgcaatataacaacataacacaacgcccagaacgaaataaaaaaaataaaagaaatagatgaaagcattttcaatttgcataccggaaaccgtaaatcaattggcgtctagctaacaactgagaatgcgaatcgccaaattgttacagaaagtagcattccgttacgtgatctgtactttaacctcttggacgtaaagaatggcagaactctggctctagtgttctgcgaatgccagatcgggaaatatttcccaaaagggcaacagcggcacgaacaagaatttcgtgttaaaaatcgcgtccggcgcgaaatttttcgcggaggcatgcgacgcaaaagcgactcgaaatgtcgggagccaaatgaggctacaaggctgtgggcagattttggcgttattatggagaataaaaggaatggtagcttccatatagcgataggaaaacatatataaggtcaataggcctacatggtaatgggattgataagcttgtccttaactcttgtgtcttggaattactattgcgtacatcagcaatcatccattgtcaagaacaaaaacgataacaaaaacaatcaattatacaaataacagtaaagtaataaa

SEQ ID NO: 78 - DNA, 1000 bp, Artificial sequence
  ARG1
aggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatagacactctccccccccctccccctctgatctttcctgttgcctctttttcccccaaccaatttatcattatacacaagttctacaactactactagtaacattactacagttattataattttctattctctttttctttaagaatctatcattaacgttaatttctatatatacataactaccattatacacgctattatcgtttacatatcacatcaccgttaatgaaagatacgacaccctgtacactaacacaattaaataatcgccataaccttttctgttatctatagcccttaaagctgtttcttcgagctttttcactgcagtaattctccacatgggcccagccactgagataagagcgctatgttagtcactactgacggctctccagtcatttatgtgattttttagtgactcatgtcgcatttggcccgtttttttccgctgtcgcaacctatttccattaacggtgccgtatggaagagtcatttaaaggcaggagagagagattactcatcttcattggatcagattgatgactgcgtacggcagatagtgtaatctgagcagttgcgagacccagactggcactgtctcaatagtatattaatgggcatacattcgtactcccttgttcttgcccacagttctctctctctttacttcttgtatcttgtctccccattgtgcagcgataaggaacattgttctaatatacacggatacaaaagaaatacacataattgcataaaata

SEQ ID NO: 79 - DNA, 1000 bp, Artificial sequence
  ZPS1
cttcttgctagtatatatgacatactggtgcaaatatggcataacatcaagaggcagtcatcatatctaaaggatacagaaatggaaggattgataatgtaacaaggtaatgaacgacaatatgtaaaatgaatgaaagcctcaataatatcatacagacaaaaatcgattcccttttgtgaatttttttttttgtatcctccaggagaaattcatacctaatgagaacagtggaatcccaacacaattatctaattttttcatctatttctcatagataaaatgaaatttcaataattgaccaatattcacctggcccataatcaatcgctgtaataaaatctccaaaaagggtaaaactaaacaacttttaaaatgtgaaaaattaagtaaagaaaaagataagaataaagtccaacaagagtcaactgcttgaaattttcagctgaagtggaaaagaggtgctgatcattgagtgccaaacggaagtttgttgctgccgcaatgtcaatgaagcattaagctctgacgttttgccgtttctttttgggcagtatgcgatcaatttatgctagccaaaagaaaaaaatcgtatgcgcgctgaatcagccgtagctagctgtcgacaatgacatggcggaagcgctgtttttaaaggcttcttataattgaccttcagggttaggaccctgaaggttttttgtagccatactgcttacaagaatgtaacctctttggaggaaaaagaaattatactttcttttccagatcacgaatctgttgaagtactgttcttttccgtggcttgaaccctctacaagagatctatagacgccgcacatgctttcaaatagagctacattctggtccttcaatatatcaattagaaacgtataaaaacaagacaaaattactgtcattgggggagaacaacccatctaggaattgggattgagcaattaagatagaaaaccaaatccacacacaactactaaacatt

SEQ ID NO: 80 - DNA, 1000 bp, Artificial sequence
  FIT2
acgcaagacaacaggcaaaataatttcgtttctcagtaccgaaatgacgaaatatactgaggcaaatgcgatcatcatgcctttgcgccaagaaactcccttgtgaagaacttcaaaccgaaatgggaaaactttgagttattgacagggaatacggaggggaagatcacacttaaatccgtatgagccgcgcacataatggtattcaaatacacaagaacattcatgagctatttttcatccgtgcaaacgaatttactacaattggaccagagggcaccataactggagactttgctactgactcaacgttgatgatgcgagtagtgggtgtactgtgatttgctcatttttttttttatagaaagattcgattaatgaaagtcacaggagacatttttacatagacattccgtatatgttgcgggtatcgcggatgcggattagtgatgcctttaactacatttcatagatttctgtataccaattgaaatgagtgaagtaagctcctacagtgaaatatctgggtgctactgacgccaagccctacagcgatcggaatgcgggaacggaagttaacggggcttccagaacggcggaagcgaattgaacgaggacggcaaacaaaaacacccaaaatttcattacttagaatgaccctcaagagcagggtgcaatttatcaagcgatcattgaactaactaagttcatatcctgtataggatttaaaacaatgcaccctaagttcaaatgcaccccccctcgccccgcagcggacccttgaacagagaactgtttcgaggttcacccaattggatcacttgtataatttgtaatcgagttcggataagatgtatacgaatctaactgggtgcagtataattagcattttatattacctagcaatatatgtataaaacaggaatgtgtgcgtgcttcaggcagaattttacggtccttgtaaaaaagtctatcataaagccatcacaaaacaataata

SEQ ID NO: 81 - DNA, 1000 bp, Artificial sequence
  FIT3
tccataaacatttcctttgtctatattatccaggggcccaaaataagcccatacccacctatctagagaggctttattgggagtgacgggatatcgtatgttttcctttacctcatcaagaggccggatgaaaagggatgcattggcaaaaattcgataatactcctcattgctgacgacctcatctttgtgataatcgtggcaatactgattaattttgctaaacgttttttcgaatatttttttacttttcttcctactgtcgagaacatccttcgcgcaatgtaaccaagaccctagtgctggttcataagtgcaaagagtagcgtgccgacccttcacatccgtgtcaaatttgtgtgattccaattctttagcacaagcatcaattgctatctggtcccattgcgttcttttcttagttgatgctggttttgctaaagaacctggtgccaaatacaccaacagcagcactaatctagcgaaaagcattcgaaatctgggtgaattaaattaactgcgacaaaattggtgatgtgacataccgaaaacgctacattacacatttgccaagtaaaaaattgacgctccttttttaatgatcgctaatgtaatgaaaagtaaaaatccaattaaacgaggaagcggtaaaatatgattgctgtgtagtaatgccacagtcttcttagtcctacctatgtcctagatttttttttgcaccctgtttgttccgcaccccgctatcccatattttttgatgtatgtgaaggaaagcaatctaagggctcaagagccgagttccgccattctaggaaagggtgcaaaaaaatggggttcaattatcgttcgagatttgttatttttttttttgtaaaagttgatttatggggtatttaagccaagaactatttgaagtattaggaggtaatatgaatgttcgaacagatacaaacaagtacttccactcgcttaaaaataactaataacaataatccctaaa

SEQ ID NO: 82 - DNA, 1000 bp, Artificial sequence
  FRE5
ttcagcagtggtggttgcagtggttgaagcagaagaggtagcagcttctgagctggtagaagtactacttggagaccaggtgttgctgctgccttcaccagtccagacaaaagtagcatcttgggtgacagtcttagtgtagacgtgaccgttcttggtggcagtgatagtggtagtgatactagaaccagaaccttcatcagcagaagaagtctcagcagcagaagaagtctcagcagcagaagtggtagcggcagcagaagtggtagcggcagcagaggtttcggcggcagaagtttcggcggcagaagattcagcggcagaagtgctgctggcgtaagagtcttcaccaccccaaacaaaagtagcatcttgggtgacagtcttagtgtagacatgaccgttcttggtggcagtgatggtggtggtgatactctcagcaagagcagtagcggcaacagcagatagaaccaaagcggaagagaatttcattttagggattattgttattagttatttttaagcgagtggaagtacttgtttgtatctgttcgaacattcatattacctcctaatacttcaaatagttcttggcttaaataccccataaatcaacttttacaaaaaaaaaaataacaaatctcgaacgataattgaaccccatttttttgcaccctttcctagaatggcggaactcggctcttgagcccttagattgctttccttcacatacatcaaaaaatatgggatagcggggtgcggaacaaacagggtgcaaaaaaaaatctaggacataggtaggactaagaagactgtggcattactacacagcaatcatattttaccgcttcctcgtttaattggatttttacttttcattacattagcgatcattaaaaaaggagcgtcaattttttacttggcaaatgtgtaatgtagcgttttcggtatgtcacatcaccaattttgtcgcagttaatttaattcacccagatttcga

SEQ ID NO: 83 - DNA, 1000 bp, Artificial sequence
  CSM4
acctgcccagacatttaatatagataggtggctttgatgagaggaagttgaaggatccttcaaaagcaagtgattgtttacgtttccgttcaagtcgcagtttagctttaacaactttccattctgaaatccaactaaaatttgaatggtgtttgaattataaccaaaattgcagtccatacatattattgcgtccgatagcagtaaattcaactgcccgtaaaattgctgcttaataaaaatcaaatgttggccgttaaagtcatacagcagtaattttttgtcctctgcttgattagaataaaaaatccagtacttgctattgaaatttctaaaacttttgaaactttgtaaatcgtgggtagtaaaatagttattaatccgtacaaattccaacccacggttacttcgaaaaacatcttcaagttccttttcttcgtcaccatcatcgtcttcttcagaatagtttccaacatctccaccatcctctccgacattgctaaaggtaagctggtcgatatgttcctttatcgtatctggaagctgtgggtaatcattattcaccgtgtagttgccatacttgctcgcccgttcgtcaaatacgttctgatatctcgtgtggtaaaattgcacccccttcccatcctggtatatttgcataggtatacccatgatttcgttatgattattttctttcttgagggtgtttagcaatacattattatccttgtgatttcgcttatacacatgtaggatttcctccttatatattacaagtgtgtcaccatttccggcccaccaatagccgccgacctccaaattaccccaaataaaaaaaaagaaaactccggaaatgcggggttgagctgcaatataataataatatggcaactttagccgcgcaatcgaagatacagtaaatatagtaaaaacgtgtctgcaaatggctactttcgatccttcttcccaaaaggcaatattgcagaagaagaactagaaa

SEQ ID NO: 84 - DNA, 1000 bp, Artificial sequence
  SAM3
ttgagtttggggagaaaaggctgcttttgttccatagtgcgtcttttaaaaattttcatccttgttattatcaaactttttcctgggcggtaacctagctgaataagaacgatttggctgcaatcgccgagaaaacaaccaagccaagaattcccaaaagggatatgcttgcatccgcattgttccctcggttctactaataagaaagagtaagagacaatgtttagcgctattaagttatgctatggttacatcattttggcggactggatcatgtctgtcgagaaaaacttgaccgataattcaacgaagtattggtgcctaagctttgcatcataatatcttacaagttttgctgagaacagatagaggcataaatgaatgatttacgtcttattttttgataaggaggtatcgacgtctgccagcttaagaatatgaaatctggcgcatgatttcttcaaaataactcagatattgtcaagaacagcccactattgcttggaagtgaaaatatacgccaaaagtgcattttactttctacaattcaccacagtcttcgagatatatttatcatttaaatcacatggacacccaccgaactggttacaagctatattcgcatcacacagaatgatgtggcgtctatgctatacggcgatgttactatatcattaacctctcttttcggttccgagcgctttcggctcaagaactggggatgactaaaaaaaaagaactgtgtacgtgatttctttgtcccctgcggttgcatagacatcgctgtcgcacggcaagagcgcacacagtcatcagtctacaaaacctaacttttcaagagcaaccagtataaatattttgagccattgagggcataaagcgaaaggcacattttcaaaattggtcttaggttcatcttctgatgttatgtcgagagctctgaaaaccaattattttgaaagctaacatttcaaaaggctatttcttctgaaatatcaag

SEQ ID NO: 85 - DNA, 1000 bp, Artificial sequence
  FDH2
tgtcctcctaatatcttttatctttaatactgtaggggcgcaagtttctttttttttttttttttatgttgcgtttagtttttctcttggcaaaagtttttcgcaccccgatctttttttgcatacgtagttcactgccgctgcttacggcagcgtttcactttgtttggagaacggttgttaacttgtatttgatatggtgtcgagacaatgtcattgcaagttatataaacattgtaatacatcacctcgatgaaagagaaactggaatgatagatctctttttctcaaaatttcgttaatatgtaataataaggttcctgatgtaatttgtttttgtacaaattattttagattctggaggttcaaataaaatatatattacagccaacgattaggggggacaagacttgattacacatttttcgttggtaacttgactcttttatgaaaagaaaacattaagttgaaggtgcacgcttgaggcgctcctttttcatggtgcttagcagcagatgaaagtgtcagaagttacctattttgtcaccatttgagaataagcttgaaagaaagttgtaaccccaacttttctatcttgcacttgtttggaccaacagccaaacggcttatcccttttcttttcccttataatcgggaatttccttactaggaaggcaccgatactataactccgaatgaaaaagacatgccagtaataaaaataattgatgttatgcggaatatactattcttggattattcactgttaactaaaagttggagaaatcactctgcactgtcaatcattgaaaaaaagaacatataaaagggcacaaaatcgagtcttttttaatgagttcttgctgaggaaaatttagttaatatatcatttacataaaacatgcatattattgtgttgtactttctttattcattttaagcaggaataattacaagtattgcaacgctaatcaaatcgaaataacagctgaaaattaat

SEQ ID NO: 86 - DNA, 6903 bp, Artificial sequence
  pLA71
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcgatctgaaatgaataacaatactgacagtagatctgaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaatctagagctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttaggttttgggacgctcgaaggctttaatttggatccgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 87 - DNA, 90 bp, Artificial Sequence
  primer 895
tctcaattattattttctactcataacctcacgcaaaataacacagtcaaatcaatcaaaatgttgacaaaagcaacaaaagaacaaaaa

SEQ ID NO: 88 - DNA, 81 bp, Artificial Sequence
  primer 679
gtggagcatcgaagactggcaacatgatttcaatcattctgatcttagagcaccttggctaactcgttgtatcatcactgg

SEQ ID NO: 89 - DNA, 20 bp, Artificial Sequence
  primer 681
ttattgcttagcgttggtag

SEQ ID NO: 90 - DNA, 22 bp, Artificial Sequence
  primer 92
gagaagatgcggccagcaaaac

SEQ ID NO: 91 - DNA, 25 bp, Artificial Sequence
  primer N245
agggtagcctccccataacataaac

SEQ ID NO: 92 - DNA, 25 bp, Artificial Sequence
  primer N246
tctccaaatatatacctcttgtgtg

SEQ ID NO: 93 - DNA, 7523 bp, Artificial sequence
  pLA34
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaatgtccaatttactgcccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtccgccaccgcggtggagct

SEQ ID NO: 94 - DNA, 6924 bp, Artificial Sequence
  pLA78
gatccgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttccaattaccgtcgctcgtgatttgtttgcaaaaagaacaaaactgaaaaaacccagacacgctcgacttcctgtcttcctattgattgcagcttccaatttcgtcacacaacaaggtcctgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaagtttaaacatgtataccgtaggacagtacttggtagatagactagaagagattggtatcgataaggttttcggtgtgccaggggattacaatttgacttttctagattacattcaaaatcacgaaggactttcctggcaagggaatactaatgaactaaacgcagcatatgcagcagatggctacgcccgtgaaagaggcgtatcagctcttgttactacattcggagtgggtgaactgtcagccattaacggaacagctggtagttttgcagaacaagtccctgtcatccacatcgtgggttctccaactatgaatgtgcaatccaacaaaaagctggttcatcattccttaggaatgggtaactttcataactttagtgaaatggctaaggaagtcactgccgctacaaccatgcttactgaagagaatgcagcttcagagatcgacagagtattagaaacagccttgttggaaaagaggccagtatacatcaatcttccaattgatatagctcataaagcaatagttaaacctgcaaaagcactacaaacagagaaatcatctggtgagagagaggcacaacttgcagaaatcatactatcacacttagaaaaggccgctcaacctatcgtaatcgccggtcatgagatcgcccgtttccagataagagaaagatttgaaaactggataaaccaaacaaagttgccagtaaccaatttggcatatggcaaaggctctttcaatgaagagaacgaacatttcattggtacctattacccagctttttctgacaaaaacgttctggattacgttgacaatagtgacttcgttttacattttggtgggaaaatcattgacaattctacctcctcattttctcaaggctttaagactgaaaacactttaaccgctgcaaatgacatcattatgctgccagatgggtctacttactctgggatttctcttaacggtcttttggcagagctggaaaaactaaactttacttttgctgatactgctgctaaacaagctgaattagctgttttcgaaccacaggccgaaacaccactaaagcaagacagatttcaccaagctgttatgaactttttgcaagctgatgatgtgttggtcactgagcaggggacatcatctttcggtttgatgttggcacctctgaaaaagggtatgaatttgatcagtcaaacattatggggctccataggatacacattacctgctatgattggttcacaaattgctgccccagaaaggagacacattctatccatcggtgatggatcttttcaactgacagcacaggaaatgtccaccatcttcagagagaaattgacaccagtgatattcattatcaataacgatggctatacagtcgaaagagccatccatggagaggatgagagttacaatgatataccaacttggaacttgcaattagttgctgaaacatttggtggtgatgccgaaactgtcgacactcacaacgttttcacagaaacagacttcgctaatactttagctgctatcgatgctactcctcaaaaagcacatgtcgttgaagttcatatggaacaaatggatatgccagaatcattgagacagattggcttagccttatctaagcaaaactcttaagtttaaactaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacg

SEQ ID NO: 95 - DNA, 90 bp, Artificial Sequence
  Primer 896
ttttatatacagtataaataaaaaacccacgtaatatagcaaaaacatattgccaacaaaaattaccgtcgctcgtgatttgtttgcaaa

SEQ ID NO: 96 - DNA, 90 bp, Artificial Sequence
  Primer 897
caaactgtgtaagtttatttatttgcaacaataattcgtttgagtacactactaatggccaccttggctaactcgttgtatcatcactgg

SEQ ID NO: 97 - DNA, 28 bp, Artificial Sequence
  Primer 365
ctctatctccgctcaggctaagcaattg

SEQ ID NO: 98 - DNA, 26 bp, Artificial Sequence
  Primer 366
cagccgactcaacggcctgtttcacg

SEQ ID NO: 99 - DNA, 28 bp, Artificial Sequence
  N638
aaaagatagtgtagtagtgataaactgg

SEQ ID NO: 100 - DNA, 22 bp, Artificial Sequence
  Primer
cgataatcctgctgtcattatc

SEQ ID NO: 101 - DNA, 6761 bp, Artificial Sequence
  pLA65
gatccgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttatgaaagcattagtgtataggggcccaggccagaagttggtggaagagagacagaagccagagcttaaggaacctggtgacgctatagtgaaggtaacaaagactacaatttgcggaaccgatctacacattcttaaaggtgacgttgcgacttgtaaacccggtcgtgtattagggcatgaaggagtgggggttattgaatcagtcggatctggggttactgctttccaaccaggcgatagagttttgatatcatgtatatcgagttgcggaaagtgctcattttgtagaagaggaatgttcagtcactgtacgaccgggggttggattctgggcaacgaaattgatggtacccaagcagagtacgtaagagtaccacatgctgacacatccctttatcgtattccggcaggtgcggatgaagaggccttagtcatgttatcagatattctaccaacgggttttgagtgcggagtcctaaacggcaaagtcgcacctggttcttcggtggctatagtaggtgctggtcccgttggtttggccgccttactgacagcacaattctactccccagctgaaatcataatgatcgatcttgatgataacaggctgggattagccaaacaatttggtgccaccagaacagtaaactccacgggtggtaacgccgcagccgaagtgaaagctcttactgaaggcttaggtgttgatactgcgattgaagcagttgggatacctgctacatttgaattgtgtcagaatatcgtagctcccggtggaactatcgctaatgtcggcgttcacggtagcaaagttgatttgcatcttgaaagtttatggtcccataatgtcacgattactacaaggttggttgacacggctaccaccccgatgttactgaaaactgttcaaagtcacaagctagatccatctagattgataacacatagattcagcctggaccagatcttggacgcatatgaaacttttggccaagctgcgtctactcaagcactaaaagtcatcatttcgatggaggcttgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtg

SEQ ID NO: 102 - DNA, 83 bp, Artificial Sequence
  Primer 856
gcttatttagaagtgtcaacaacgtatctaccaacgatttgacccttttccacaccttggctaactcgttgtatcatcactgg

SEQ ID NO: 103 - DNA, 79 bp, Artificial Sequence
  Primer 857
gcacaatatttcaagctataccaagcatacaatcaactatctcatatacaatgaaagcattagtgtataggggcccagg

SEQ ID NO: 104 - DNA, 80 bp, Artificial Sequence
  BK415
gcacaatatttcaagctataccaagcatacaatcaactatctcatatacaatgaaagcattagtgtataggggcccaggc

SEQ ID NO: 105 - DNA, 26 bp, Artificial Sequence
  N1092
agagttttgatatcatgtatatcgag

SEQ ID NO: 106 - DNA, 25 bp, Artificial Sequence
  Primer 413
ggacataaaatacacaccgagattc

SEQ ID NO: 107 - DNA, 87 bp, Artificial Sequence
  Primer 906
aaaaagattcaatgccgtctcctttcgaaacttaataatagaacaatatcatccttcaccttggctaactcgttgtatcatcactgg

SEQ ID NO: 108 - DNA, 70 bp, Artificial Sequence
  Primer 907
tctcctttcgaaacttaataatagaacaatatcatccttttgtaaaacgacggccagtgaattcaccttg

SEQ ID NO: 109 - DNA, 25 bp, Artificial Sequence
  Primer 749
caagtcttttgtgccttcccgtcgg

SEQ ID NO: 110 - DNA, 4242 bp, Artificial Sequence
  pLA59
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcaatgcggatccgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 111 - DNA, 90 bp, Artificial Sequence
  LA512
gtattttggtagattcaattctctttccctttccttttccttcgctccccttccttatcagcattgcggattacgtattctaatgttcag

SEQ ID NO: 112 - DNA, 90 bp, Artificial Sequence
  LA513
ttggttgggggaaaaagaggcaacaggaaagatcagagggggagggggggggagagtgtcaccttggctaactcgttgtatcatcactgg

SEQ ID NO: 113 - DNA, 29 bp, Artificial Sequence
  LA516
ctcgaaacaataagacgacgatggctctg

SEQ ID NO: 114 - DNA, 20 bp, Artificial Sequence
  LA135
cttggcagcaacaggactag

SEQ ID NO: 115 - DNA, 30 bp, Artificial Sequence
  LA514
cactatctggtgcaaacttggcaccggaag

SEQ ID NO: 116 - DNA, 29 bp, Artificial Sequence
  LA515
tgtttgtagccactcgtgaacttctctgc

SEQ ID NO: 117 - DNA, 7223 bp, Artificial Sequence
  pBP3836
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 118 - DNA, 7398 bp, Artificial Sequence
  pBP3840
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgacagcgcaaaggattatgacactgttgcattgagtcaaaagtttttccgaagtgacccagtgctctttttttttttccgtgaaggactgacaaatatgcgcacaagatccaatacgtaatggaaattcggaaaaactaggaagaaatgctgcagggcattgccgtgcgcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 119 - DNA, 7271 bp, Artificial Sequence
  pBP3933
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgactgtttgtttgaagagactaatcaaagaatcgttttctcaaaaaatttaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaggcagaggagagcataaaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacaacactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 120 - DNA, 7560 bp, Artificial Sequence
  pBP3935
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgactcaatgcttctttcttgcgttctttttgttgcgcccatatattattttatttattacattcatatagcaatatttaccttattttattggttacttttctatacgcaaaatcactacactatgttatgttaaggtctccgatacgggaatataccaatcaatacttatcacttcggattttttatgggtcttatccccactgttccattttcttgtttaaggcatcccggaggataaactaaaaaggtggcccatcccacccgaaatgaaagtaatcatctgctagcaaaaagtaaagaaatgagagcatgctgtgatgtactggtggacgaaattgtgacccatacccaccgaagaaacatccgcatgacgtgttactgttacttcccggattaagggatgcattctaactctgtgcgcccttttctctgcagttgatccgcattccccgtggctgtgcacattaggggacagtaagtaattcgctttctgattccgcactcatagcgatggaataatataccggatttcacaccttgctattgagtgaagtactgcttggtgaaatgatatctttatgttcaatattaatggtcgtgtggatgaatatatgggcatgggttaattagttttaggggcacggagtaaacaagaaaggagggccagaatcattagtacagtacctcaagtttgatttctttttgatttcacgtataaaagagtctctctcttttcctttcatgctagtcgaacggttctccctctcagaataagaaactatcgaaaagaaagacaaaagtcgattgaataatttatctatatataatatacgcaaacaagattcgctttcactttgcaattttacttcatagctttgttaaaaccagcaaaaaatattatttttctagaaaaaagaatatattagaggtaaagaaagaactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 121 - DNA, 7622 bp, Artificial Sequence
  pBP3937
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacaatagtactctcatcgctaagatcatttggggttgttaagcatgccctgctaaacacgccctactaaacacttcaaaagcaacttaaaatatttttatctaattatagctaaaacccaatgtgaaagacatatcatactgtaaaagtgaaaaagcagcaccgttgaacgccgcaagagtgctcccataacgctttactagagggctagattttaatggccccttcatggagaagttatgaggacaaatcccactacagaaagcgcaacaaatttttttttccgtaacaacaaacatctcatctagtttctgccttaaacaaagccgcagccagagccgtttttccgccatatttatccaggattgttccatacggctccgtcagaggctgctacgggatgttttttttttaccccgtggaaatgaggggtatgcaggaatttgtgcggggtaggaaatcttttttttttttaggaggaacaactggtggaagaatgcccacacttctcagaaatgcatgcagtggcagcacgctaattcgaaaaaattctccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaaactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 122 - DNA, 7572 bp, Artificial Sequence
  pBP3940
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacagcctccccataacataaactcaataaaatatatagtcttcaacttgaaaaaggaacaagctcatgcaaagaggtggtacccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattatcttctactcataacctcacgcaaaataacacagtcaaatcaatcaaaactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 123 - DNA, 12319 bp, Artificial Sequence
  pLH689::I2V5
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgcggaggagtggaaaagagctgaagaacaaggtttcgaagtctacaccgctgctgaagctgctaagaaggctgacatcattatgatcttgatcccagatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaactgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctcgcgaactccaaaatgagctatcaaaaacgatagatcgattaggatgactttgaaatgactccgcagtggactggccgttaatttcaagcgtgagtaaaatagtgcatgacaaaagatgagctaggcttttgtaaaaatatcttacgttgtaaaattttagaaatcattatttccttcatatcattttgtcattgaccttcagaagaaaagagccgaccaataatataaataaataaataaaaataatattccattatttctaaacagattcaatactcattaaaaaactatatcaattaatttgaattaacgcggccgcttaaccacagcaaccaggacaacattttttgccagtttcttcaggcttccaaaagtctgttacggctcccctagaagcagacgaaacgatgtgagcatatttaccaaggataccgcgtgaatagagcggtggcaattcaatggtctcttgacgatgttttaactcttcatcggagatatcaaagtgtaattccttagtgtcttggtcaatagtgactatgtctcctgtttgcaggtaggcgattggaccgccatcttgtgcttcaggagcgatatgacccacgacaagaccataagtaccacctgagaagcggccatctgtcagaagggcaactttttcaccttgccctttaccaacaatcattgatgaaagggaaagcatttcaggcataccaggaccgccctttggtcctacaaaacgtacgacaacaacatcaccatcaacaatatcatcattcaagacagcttcaatggcttcttcttcagaattaaagaccttagcaggaccgacatgacgacgcacttttacaccagaaactttggcaacggcaccgtctggagccaagttaccatggagaacaatgagcggaccatcttcacgtttaggattttcaagcggcataataaccttttgaccaggtgttaaatcatcaaaagccttcaaattttcagcgactgttttgccagtacaagtgatacggtcaccatgaaggaagccatttttaaggagatatttcataactgctggtacccctccgaccttgtaaaggtcttggaatacatattgaccagaaggtttcaaatcagccaaatgaggaactttttcttggaaagtattgaaatcatcaagtgtcaattccacattagcagcatgggcaatagctaagaggtgaagggttgagttggttgaacctcccagagccatagttacagtaatagcatcttcaaaagcttcacgcgttaaaatgtcagaaggttttaagcccatttcgagcattttgacaacagcgcgaccagcttcttcaatatctgctttcttttctgcggattcagccgggtgagaagatgaacccggaaggctaagtcccaaaacttcaatagctgtcgccattgtgttagcagtatacataccaccgcagcctccaggaccgggacaagcattacattccaaagctttaacttcttctttggtcatatcgccgtggttccaatggccgacaccttcaaagacagagactaaatcgatatctttgccgtctaaattaccaggtgcaattgttccgccgtaagcaaaaatggctgggatatccatgttagccatagcgataacagaaccgggcatgtttttatcacaaccgccaatggctacaaaagcatccgcattatgacctcccatggctgcttcaatagaatctgcaataatatcacgagatgtcaaggagaaacgcattccttgggttcccatggcgattccatcagaaaccgtgattgttccgaactgaactggccaagcaccagcttccttaacaccgactttggctagtttaccaaagtcatgtaagtggatattacaaggtgtgttttcagcccaagttgaaatgacaccgacgataggtttttcaaagtcttcatcttgcataccagttgcacgcaacatagcacgattaggtgatttaaccattgaatcgtaaacagaactacgatttcttaagtctttaagagtttttttgtcagtcatactcacgtgaaacttagattagattgctatgctttctttccaatgagcaagaagtaaaaaaagttgtaatagaacaggaaaaatgaagctgaaacttgagaaattgaagaccgtttgttaactcaaatatcaatgggaggtcgtcgaaagagaacaaaatcgaaaaaaaagttttcaagagaaagaaacgtgataaaaatttttattgccttctccgacgaagaaaaagggacgaggcggtctctttttccttttccaaacctttagtacgggtaattaacggcaccctagaggaaggaggagggggaatttagtatgctgtgcttgggtgttttgaagtggtacggcggtgcgcggagtccgagaaaatctggaagagtaaaaaaggagtagagacattttgaagctatgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 124 - DNA, 18 bp, Artificial Sequence
  HAP4-32F
ccgctagtcgccctcgta

SEQ ID NO: 125 - DNA, 20 bp, Artificial Sequence
  HAP4-157R
tgccatcgttttcgaattcc

SEQ ID NO: 126 - DNA, 20 bp, Artificial Sequence
  HAP4-89T
cgcctgtaccgatcgcccca

SEQ ID NO: 127 - DNA, 18 bp, Artificial Sequence
  CYC1-64F
caatgccacaccgtggaa

SEQ ID NO: 128 - DNA, 22 bp, Artificial Sequence
  CYC1-130R
tgccaaagataccatgcaagtt

SEQ ID NO: 129 - DNA, 25 bp, Artificial Sequence
  CYC1-83T
agggtggcccacataaggttggtcc

SEQ ID NO: 130 - DNA, 20 bp, Artificial Sequence
  PDA1-11F
cttcattcaaacgccaacca

SEQ ID NO: 131 - DNA, 19 bp, Artificial Sequence
  PDA1-75R
ggtgggagtgcgaagaaca

SEQ ID NO: 132 - DNA, 24 bp, Artificial Sequence
  PDA1-32T
cacaattggtccgcgggttaggag

SEQ ID NO: 133 - DNA, 19 bp, Artificial Sequence
  MDH1-329F
ccatcaacgcaagcatcgt

SEQ ID NO: 134 - DNA, 18 bp, Artificial Sequence
  MDH1-391R
cagcattgggagcggatt

SEQ ID NO: 135 - DNA, 21 bp, Artificial Sequence
  MDH1-351T
cgatttggcagcagcaaccgc

SEQ ID NO: 136 - DNA, 21 bp, Artificial Sequence
  NDE1-1263F
tgctatcggcgattgtacctt

SEQ ID NO: 137 - DNA, 21 bp, Artificial Sequence
  NDE1-1329R
accttcttggtgggcaacttg

SEQ ID NO: 138 - DNA, 20 bp, Artificial Sequence
  NDE1-1288T
cctggcttgttccctaccgc

SEQ ID NO: 139 - DNA, 21 bp, Artificial Sequence
  18S-396F
agaaacggctaccacatccaa

SEQ ID NO: 140 - DNA, 25 bp, Artificial Sequence
  18S-468R
tcactacctccctgaattaggattg

SEQ ID NO: 141 - DNA, 21 bp, Artificial Sequence
  18S-420T
aaggcagcaggcgcgcaaatt

SEQ ID NO: 142 - DNA, 7505 bp, Saccharomyces cerevisiae
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcggccgcggcgcgccttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaagtttaaacatgaccgcaaagacttttctactacaggcctccgctagtcgccctcgtagtaaccattttaaaaatgagcataataatattccattggcgcctgtaccgatcgccccaaataccaaccatcataacaatagttcgctggaattcgaaaacgatggcagtaaaaagaagaagaagtctagcttggtggttagaacttcaaaacattgggttttgcccccaagaccaagacctggtagaagatcatcttctcacaacactctacctgccaacaacaccaataatattttaaatgttggccctaacagcaggaacagtagtaataataataataataataacatcatttcgaataggaaacaagcttccaaagaaaagaggaaaataccaagacatatccagacaatcgatgaaaagctaataaacgactcgaattacctcgcatttttgaagttcgatgacttggaaaatgaaaagtttcattcttctgcctcctccatttcatctccatcttattcatctccatctttttcaagttatagaaatagaaaaaaatcagaattcatggacgatgaaagctgcaccgatgtggaaaccattgctgctcacaacagtctgctaacaaaaaaccatcatatagattcttcttcaaatgttcacgcaccacccacgaaaaaatcaaagttgaacgactttgatttattgtccttatcttccacatcttcatcggccactccggtcccacagttgacaaaagatttgaacatgaacctaaattttcataagatccctcataaggcttcattccctgattctccagcagatttctctccagcagattcagtctcgttgattagaaaccactccttgcctactaatttgcaagttaaggacaaaattgaggatttgaacgagattaaattctttaacgatttcgagaaacttgagtttttcaataagtatgccaaagtcaacacgaataacgacgttaacgaaaataatgatctctggaattcttacttacagtctatggacgatacaacaggtaagaacagtggcaattaccaacaagtggacaatgacgataatatgtctttattgaatctgccaattttggaggaaaccgtatcttcagggcaagatgataaggttgagccagatgaagaagacatttggaattatttaccaagttcaagttcacaacaagaagattcatcacgtgctttgaaaaaaaatactaattctgagaaggcgaacatccaagcaaagaacgatgaaacctatctgtttcttcaggatcaggatgaaagcgctgattcgcatcaccatgacgagttaggttcagaaatcactttggctgacaataagttttcttatttgcccccaactctagaagagttgatggaagagcaggactgtaacaatggcagatcttttaaaaatttcatgttttccaacgataccggtattgacggtagtgccggtactgatgacgactacaccaaagttctgaaatccaaaaaaatttctacgtcgaagtcgaacgctaacctttatgacttaaacgataacaacaatgatgcaactgccaccaatgaacttgatcaaagcagtttcatcgacgaccttgacgaagatgtcgattttttaaaggtacaagtattttgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtcgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 143 - DNA, 6005 bp, Saccharomyces cerevisiae
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcggccgcggcgcgccgcttgcatttagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaatagtttaaacttaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtcgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 144 - DNA, 12298 bp, Saccharomyces cerevisiae
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgcggaggagtggaaaagagctgaagaacaaggtttcgaagtctacaccgctgctgaagctgctaagaaggctgacatcattatgatcttgatcccagatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaactgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctcgcgaactccaaaatgagctatcaaaaacgatagatcgattaggatgactttgaaatgactccgcagtggactggccgttaatttcaagcgtgagtaaaatagtgcatgacaaaagatgagctaggcttttgtaaaaatatcttacgttgtaaaattttagaaatcattatttccttcatatcattttgtcattgaccttcagaagaaaagagccgaccaataatataaataaataaataaaaataatattccattatttctaaacagattcaatactcattaaaaaactatatcaattaatttgaattaacttaattaattattttttgccagtttcttcaggcttccaaaagtctgttacggctcccctagaagcagacgaaacgatgtgagcatatttaccaaggataccgcgtgaatagagcggtggcaattcaatggtctcttgacgatgttttaactcttcatcggagatatcaaagtgtaattccttagtgtcttggtcaatagtgactatgtctcctgtttgcaggtaggcgattggaccgccatcttgtgcttcaggagcgatatgacccacgacaagaccataagtaccacctgagaagcggccatctgtcagaagggcaactttttcaccttgccctttaccaacaatcattgatgaaagggaaagcatttcaggcataccaggaccgccctttggtcctacaaaacgtacgacaacaacatcaccatcaacaatatcatcattcaagacagcttcaatggcttcttcttcagaattaaagaccttagcaggaccgacatgacgacgcacttttacaccagaaactttggcaacggcaccgtctggagccaagttaccatggagaataatgaccggaccatcttcacgtttaggattttcaagcggcataataaccttttgaccaggtgttaaatcatcaaaagccttcaaattttcagcgactgttttgccagtacaagtgatacggtcaccatgaaggaagccatttttaaggagatatttcataactgctggtacccctccgaccttgtaaaggtcttggaatacatattgaccagaaggtttcaaatcagccaaatgaggaactttttcttggaaagtattgaaatcatcaagtgtcaattccacattagcagcatgggcaatagctaagaggtgaagggttgagttggttgaacctcccagagccatagttacagtaatagcatcttcaaaagcttcacgcgttaaaatgtcagaaggttttaagcccatttcgagcattttgacaacagcgcgaccagcttcttcaatatctgctttcttttctgcggattcagccgggtgagaagatgaacccggaaggctaagtcccaaaacttcaatagctgtcgccattgtgttagcagtatacataccaccgcagcctccaggaccgggacaagcattacattccaaagctttaacttcttctttggtcatatcgccgtggttccaatggccgacaccttcaaagacagagactaaatcgatatctttgccgtctaaattaccaggtgcaattgttccgccgtaagcaaaaatggctgggatatccatgttagccatagcgataacagaaccgggcatgtttttatcacaaccgccaatggctacaaaagcatccgcattatgacctcccatggctgcttcaatagaatctgcaataatatcacgagatgtcaaggagaaacgcattccttgggttcccatggcgattccatcagaaaccgtgattgttccgaactgaactggccaagcaccagcttccttaacaccgactttggctagtttaccaaagtcatgtaagtggatattacaaggtgtgttttcagcccaagttgaaatgacaccgacgataggtttttcaaagtcttcatcttgcataccagttgcacgcaacatagcacgattaggtgatttaaccattgaatcgtaaacagaactacgatttcttaagtctttaagagtttttttgtcagtcatactcacgtgaaacttagattagattgctatgctttctttccaatgagcaagaagtaaaaaaagttgtaatagaacaggaaaaatgaagctgaaacttgagaaattgaagaccgtttgttaactcaaatatcaatgggaggtcgtcgaaagagaacaaaatcgaaaaaaaagttttcaagagaaagaaacgtgataaaaatttttattgccttctccgacgaagaaaaagggacgaggcggtctctttttccttttccaaacctttagtacgggtaattaacggcaccctagaggaaggaggagggggaatttagtatgctgtgcttgggtgttttgaagtggtacggcggtgcgcggagtccgagaaaatctggaagagtaaaaaaggagtagagacattttgaagctatgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 145 - DNA, 7564 bp, Saccharomyces cerevisiae
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcggccgcggcgcgcctgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacagtttaaacatgaccgcaaagacttttctactacaggcctccgctagtcgccctcgtagtaaccattttaaaaatgagcataataatattccattggcgcctgtaccgatcgccccaaataccaaccatcataacaatagttcgctggaattcgaaaacgatggcagtaaaaagaagaagaagtctagcttggtggttagaacttcaaaacattgggttttgcccccaagaccaagacctggtagaagatcatcttctcacaacactctacctgccaacaacaccaataatattttaaatgttggccctaacagcaggaacagtagtaataataataataataataacatcatttcgaataggaaacaagcttccaaagaaaagaggaaaataccaagacatatccagacaatcgatgaaaagctaataaacgactcgaattacctcgcatttttgaagttcgatgacttggaaaatgaaaagtttcattcttctgcctcctccatttcatctccatcttattcatctccatctttttcaagttatagaaatagaaaaaaatcagaattcatggacgatgaaagctgcaccgatgtggaaaccattgctgctcacaacagtctgctaacaaaaaaccatcatatagattcttcttcaaatgttcacgcaccacccacgaaaaaatcaaagttgaacgactttgatttattgtccttatcttccacatcttcatcggccactccggtcccacagttgacaaaagatttgaacatgaacctaaattttcataagatccctcataaggcttcattccctgattctccagcagatttctctccagcagattcagtctcgttgattagaaaccactccttgcctactaatttgcaagttaaggacaaaattgaggatttgaacgagattaaattctttaacgatttcgagaaacttgagtttttcaataagtatgccaaagtcaacacgaataacgacgttaacgaaaataatgatctctggaattcttacttacagtctatggacgatacaacaggtaagaacagtggcaattaccaacaagtggacaatgacgataatatgtctttattgaatctgccaattttggaggaaaccgtatcttcagggcaagatgataaggttgagccagatgaagaagacatttggaattatttaccaagttcaagttcacaacaagaagattcatcacgtgctttgaaaaaaaatactaattctgagaaggcgaacatccaagcaaagaacgatgaaacctatctgtttcttcaggatcaggatgaaagcgctgattcgcatcaccatgacgagttaggttcagaaatcactttggctgacaataagttttcttatttgcccccaactctagaagagttgatggaagagcaggactgtaacaatggcagatcttttaaaaatttcatgttttccaacgataccggtattgacggtagtgccggtactgatgacgactacaccaaagttctgaaatccaaaaaaatttctacgtcgaagtcgaacgctaacctttatgacttaaacgataacaacaatgatgcaactgccaccaatgaacttgatcaaagcagtttcatcgacgaccttgacgaagatgtcgattttttaaaggtacaagtattttgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtcgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 146 - DNA, 7915 bp, Saccharomyces cerevisiae
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcggccgcggcgcgccaatagtactctcatcgctaagatcatttggggttgttaagcatgccctgctaaacacgccctactaaacacttcaaaagcaacttaaaatatttttatctaattatagctaaaacccaatgtgaaagacatatcatactgtaaaagtgaaaaagcagcaccgttgaacgccgcaagagtgctcccataacgctttactagagggctagattttaatggccccttcatggagaagttatgaggacaaatcccactacagaaagcgcaacaaatttttttttccgtaacaacaaacatctcatctagtttctgccttaaacaaagccgcagccagagccgtttttccgccatatttatccaggattgttccatacggctccgtcagaggctgctacgggatgttttttttttaccccgtggaaatgaggggtatgcaggaatttgtgcggggtaggaaatcttttttttttttaggaggaacaactggtggaagaatgcccacacttctcagaaatgcatgcagtggcagcacgctaattcgaaaaaattctccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaagtttaaacatgaccgcaaagacttttctactacaggcctccgctagtcgccctcgtagtaaccattttaaaaatgagcataataatattccattggcgcctgtaccgatcgccccaaataccaaccatcataacaatagttcgctggaattcgaaaacgatggcagtaaaaagaagaagaagtctagcttggtggttagaacttcaaaacattgggttttgcccccaagaccaagacctggtagaagatcatcttctcacaacactctacctgccaacaacaccaataatattttaaatgttggccctaacagcaggaacagtagtaataataataataataataacatcatttcgaataggaaacaagcttccaaagaaaagaggaaaataccaagacatatccagacaatcgatgaaaagctaataaacgactcgaattacctcgcatttttgaagttcgatgacttggaaaatgaaaagtttcattcttctgcctcctccatttcatctccatcttattcatctccatctttttcaagttatagaaatagaaaaaaatcagaattcatggacgatgaaagctgcaccgatgtggaaaccattgctgctcacaacagtctgctaacaaaaaaccatcatatagattcttcttcaaatgttcacgcaccacccacgaaaaaatcaaagttgaacgactttgatttattgtccttatcttccacatcttcatcggccactccggtcccacagttgacaaaagatttgaacatgaacctaaattttcataagatccctcataaggcttcattccctgattctccagcagatttctctccagcagattcagtctcgttgattagaaaccactccttgcctactaatttgcaagttaaggacaaaattgaggatttgaacgagattaaattctttaacgatttcgagaaacttgagtttttcaataagtatgccaaagtcaacacgaataacgacgttaacgaaaataatgatctctggaattcttacttacagtctatggacgatacaacaggtaagaacagtggcaattaccaacaagtggacaatgacgataatatgtctttattgaatctgccaattttggaggaaaccgtatcttcagggcaagatgataaggttgagccagatgaagaagacatttggaattatttaccaagttcaagttcacaacaagaagattcatcacgtgctttgaaaaaaaatactaattctgagaaggcgaacatccaagcaaagaacgatgaaacctatctgtttcttcaggatcaggatgaaagcgctgattcgcatcaccatgacgagttaggttcagaaatcactttggctgacaataagttttcttatttgcccccaactctagaagagttgatggaagagcaggactgtaacaatggcagatcttttaaaaatttcatgttttccaacgataccggtattgacggtagtgccggtactgatgacgactacaccaaagttctgaaatccaaaaaaatttctacgtcgaagtcgaacgctaacctttatgacttaaacgataacaacaatgatgcaactgccaccaatgaacttgatcaaagcagtttcatcgacgaccttgacgaagatgtcgattttttaaaggtacaagtattttgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtcgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

(19) United States
(12) Patent Application Publication
(10) Pub. No.: US 2016/0326552 A1
(43) Pub. Date: Nov. 10, 2016

(54) EXPRESSION OF A HAP TRANSCRIPTIONAL COMPLEX SUBUNIT

(71) Applicant: BUTAMAX ADVANCED BIOFUELS LLC, Wilmington, DE (US)

(72) Inventors: Michael DAUNER, Wilmington, DE (US); Brian James PAUL, Wilmington, DE (US)

(21) Appl. No.: 15/108,882

(22) PCT Filed: Dec. 22, 2014
(86) PCT No.: PCT/US14/71903

§ 371 Date: Jun. 29, 2016

Related U.S. Application Data
(60) Provisional application No. 61/922,593, filed on Dec. 31, 2013.

(51) Int. Cl. C12P 7/16; C12N 9/88; C12N 9/04; C07K 14/395

(52) U.S. Cl. CPC C12P 7/16; C07K 14/395; C12N 9/88; C12N 9/0006; C12Y 101/01008; C12Y 401/01001

(57) ABSTRACT

The invention relates, for example, to recombinant yeast cells for differential gene expression during the propagation and production phases of a fermentation-based production process, as well as methods for using the same.

### CROSS REFERENCE TO RELATED APPLICATION

This application claims benefit of priority from U.S. Provisional Application No. 61/922,593, filed Dec. 31, 2013, which is hereby incorporated by reference in its entirety.

### REFERENCE TO SEQUENCE LISTING SUBMITTED ELECTRONICALLY

The content of the electronically submitted sequence listing in ASCII text file (Name: 20141212_CL6087WPPCT_SequenceListing_ascii.txt; Size: 398,151 bytes; and Date of Creation: Dec. 4, 2013) filed with the application is incorporated herein by reference in its entirety.

### FIELD OF THE INVENTION

The invention relates to the fields of industrial microbiology and higher alcohol production. Embodiments of the invention relate to recombinant yeast cells for use in differential regulation of the expression of genes during propagation and production phases to achieve, for example, increased growth rate, μcrit, and/or biomass via an engineered pathway in the recombinant yeast cell, as well as methods for using the same.

### BACKGROUND OF THE INVENTION

Technologies which allow utilization of renewable resources instead of fossil fuels for production of useful materials will mitigate depletion of oil reserves and minimize net CO₂ emissions. Thus, there is a need for materials and processes for efficient conversion of plant derived raw materials to a valuable product stream, for example liquid transportation fuel.

Under high glucose concentrations, S. cerevisiae naturally produces ethanol under aerobic conditions. This phenomenon is known as the Crabtree effect. However, the formation of ethanol under aerobic conditions can be overcome by growing yeast cells under conditions of sugar limitation, usually a fed-batch regime. Nevertheless, if the cells grow faster than a critical growth rate (“μcrit”), even under glucose-limited conditions, the ethanol formation commences leading to lower biomass yields and the accumulation of ethanol. Because industrial production with yeast may employ a stage of biomass production in order to provide appropriate mass of biocatalyst for desired yield and production rate, it may be desirable to optimize biomass production on the substrate.

### BRIEF SUMMARY OF THE INVENTION

Provided herein is a recombinant yeast cell. In embodiments, the recombinant yeast cell comprises (a) a recombinant polynucleotide encoding a gene for a subunit of the HAP transcriptional complex; and (b) an engineered higher alcohol biosynthetic pathway. In embodiments, the subunit of the HAP transcriptional complex is Hap2, Hap3, Hap4, or Hap5. In embodiments, the subunit of the Hap transcriptional complex comprises an amino acid sequence at least 80%, at least 85%, at least 90%, at least 95%, at least 96%, at least 97%, at least 98%, or at least 99% identical to any of SEQ ID NOs:2, 4, 6, or 8. In embodiments, the polynucleotide comprises a nucleic acid sequence at least 80%, at least 85%, at least 90%, at least 95%, at least 96%, at least 97%, at least 98% or at least 99% identical to any of SEQ ID NOs:1, 3, 5, or 7. In embodiments, the gene is expressed during propagation phase of a fermentation-based production process. In embodiments, the gene is down-regulated or not expressed during production phase of a fermentation-based production process. In embodiments, the gene is operably linked to a conditional promoter. In embodiments, the activity of the conditional promoter is greater during propagation phase of a fermentation-based production process when compared to during production phase. In embodiments, the conditional promoter is ADH2, HXT5 or HXT7. In embodiments, the conditional promoter comprises a nucleic acid sequence at least 80%, at least 85%, at least 90%, at least 95%, at least 96%, at least 97%, at least 98% or at least 99% identical to any of SEQ ID NOs:9, 10, or 11. In embodiments, the recombinant yeast cell further comprises (c) at least one genetic modification that reduces or eliminates activity of an endogenous pyruvate decarboxylase. In embodiments, the at least one genetic modification that reduces or eliminates activity of an endogenous pyruvate decarboxylase is a deletion, disruption, or mutation in an endogenous gene encoding pyruvate decarboxylase. In embodiments, the pyruvate decarboxylase is PDC1, PDC5, PDC6 or combination thereof. In embodiments, the engineered higher alcohol biosynthetic pathway is an isobutanol biosynthetic pathway, a butanol biosynthetic pathway, or a 2-butanone biosynthetic pathway. In embodiments, the isobutanol biosynthetic pathway comprises one or more of (a) at least one genetic construct encoding an acetolactate synthase; (b) at least one genetic construct encoding acetohydroxy acid isomeroreductase; (c) at least one genetic construct encoding acetohydroxy acid dehydratase; (d) at least one genetic construct encoding branched-chain keto acid decarboxylase; and (e) at least one genetic construct encoding branched-chain alcohol dehydrogenase. In embodiments, the yeast is from the genus Saccharomyces, Schizosaccharomyces, Hansenula, Kluyveromyces, Candida, Pichia, or Yarrowia. In embodiments, the yeast is Saccharomyces cerevisiae, Schizosaccharomyces pombe, Kluyveromyces lactis, Kluyveromyces thermotolerans, Candida glabrata, Candida albicans, Pichia stipitis, or Yarrowia lipolytica. In embodiments, the recombinant yeast cell has at least a 10% improvement in growth rate. In embodiments, the recombinant yeast cell has at least a 10% improvement in maximum specific growth rate.

Also provided herein is a method for generating a recombinant yeast cell, comprising introducing into a yeast cell (a) a recombinant polynucleotide encoding a gene for a subunit of the HAP transcriptional complex, and (b) an engineered higher alcohol biosynthetic pathway.

Also provided herein is a method for increasing maximum specific growth rate of a yeast cell, comprising introducing into a yeast cell (a) a recombinant polynucleotide encoding a gene for a subunit of the HAP transcriptional complex, and (b) an engineered higher alcohol biosynthetic pathway; wherein growth rate of the yeast cell is greater when compared to growth rate or maximum specific growth rate of a yeast cell that does not contain a recombinant polynucleotide encoding a gene for a subunit of the HAP transcriptional complex. In some embodiments, overexpression of Hap4p would occur during the biocatalyst production phase, and Hap4p expression would be down-regulated or even completely abolished during the butanol production phase. This controlled mode of expression can be realized e.g. with the help of a “genetic switch”, in particular with promoters that are “on” or highly expressed during the biocatalyst production phase, and “off” or expressed at low levels during the butanol production phase. For example, promoters are regulated by the presence of glucose. Promoters, such as the Saccharomyces cerevisiae ADH2, HXT5, and HXT7 promoters, are “on” or highly expressed during glucose limitation, and “off” or expressed at low levels during glucose excess.

In embodiments, the gene is expressed during propagation phase of a fermentation-based production process. In embodiments, the gene is down-regulated or not expressed during production phase of a fermentation-based production process. In embodiments, the gene is operably linked to a conditional promoter. In embodiments, the activity of the conditional promoter is higher during a propagation phase of a fermentation-based production process when compared to during production phase. In embodiments, the conditional promoter is ADH2, HXT5 or HXT7. In embodiments, the conditional promoter comprises a nucleic acid sequence at least 80%, at least 85%, at least 90%, at least 95%, at least 96%, at least 97%, at least 98% or at least 99% identical to any of SEQ ID NOs:9, 10, or 11. In embodiments, the fermentation-based production process is under aerobic conditions. In embodiments, the fermentation-based production process is under anaerobic or microaerobic conditions. In embodiments, glucose is present during the fermentation-based production process. In embodiments, glucose is not present during the fermentation-based production process. In embodiments, ethanol is present during the fermentation-based production process. In embodiments, sodium acetate is present during the fermentation-based production process.

Also provided herein is a method for production of isobutanol, comprising (a) providing a recombinant yeast cell; and (b) culturing the cell of (a) under conditions wherein isobutanol is produced. In embodiments, the method further comprises (c) recovering the isobutanol

### BRIEF DESCRIPTION OF THE FIGURES

FIG. 1A-1B depict the effect on growth rate of overexpressing HAP4 in yeast strains in the presence of glucose with or without ethanol compared to a control strain.

FIG. 2A-2B depict the effect on growth rate of overexpressing HAP4 in yeast strains in the presence of low glucose with or without ethanol compared to a control strain.

FIG. 3A-3B depict the growth rates of yeast strains overexpressing HAP4 compared to a control strain with only ethanol as the carbon source.

FIG. 4 shows the growth of yeast strains overexpressing HAP4 compared to a control strain in serum vials.

FIG. 5 shows the amount of glucose consumed and isobutanol produced by yeast strains overexpressing HAP4 compared to a control strain.

FIG. 6 shows the isobutanol molar yield for yeast strains overexpressing HAP4 or a control strain.

FIG. 7A-7F show the effect of addition of 3% glucose on promoter-GFP fusions in yeast strains PNY1631, PNY1632, PNY1633, PNY1634, PNY1635, and PNY1636.

FIG. 8 demonstrates the overexpression of HAP4 mRNA in PNY1650/PNY1651 (HAP4) and its effect on the expression of select genes compared to PNY1648/PNY1649 (control) in the presence of glucose with or without ethanol.

FIG. 9 shows the average and standard deviation of relative mRNA expression of HAP4 and CYC1 in yeast strains overexpressing HAP4 with different promoters in high glucose or low glucose conditions compared to a control strain.

FIG. 10A-10D show the growth rates of yeast strains overexpressing HAP4 with different promoters compared to a control strain under low and high glucose conditions in the presence of ethanol.

FIG. 11A-11B show the average growth rate and standard deviation for yeast strains overexpressing HAP4 with the FBA1 or ADH2 promoter compared to a control strain in the presence of sodium acetate.

FIG. 12 shows the serum vial growth of yeast strains overexpressing HAP4 with the FBA1 or ADH2 promoter compared to a control strain.

FIG. 13 shows glucose consumed and isobutanol produced by yeast strains overexpressing HAP4 with the FBA1 or ADH2 promoter compared to a control strain.

FIG. 14 shows the isobutanol molar yield for yeast strains overexpressing HAP4 with the FBA1 or ADH2 promoter compared to a control strain.

### DETAILED DESCRIPTION OF THE INVENTION

The invention is directed to recombinant yeast cells that comprise a recombinant polynucleotide encoding a gene for a subunit of the HAP transcriptional complex. The cells further comprise promoter sequences that provide differential expression in the propagation vs. production phases of a process, as well as methods for using the same. In embodiments, the cells have increased growth rate, μcrit and/or biomass production. In other embodiments, the cells produce a fermentation product.

Native S. cerevisiae is a Crabtree-positive yeast, in which the fraction of respiratory metabolism on overall metabolism is negatively correlated with increasing extracellular glucose concentration. For example, when glucose concentration is high, many genes involved in respiration, gluconeogenesis and utilization of non-glucose carbon sources are expressed at low levels or not at all. Therefore, in S. cerevisiae alcoholic fermentation occurs even under aerobic conditions if glucose concentration exceeds a certain value and at high growth rate. In industrial processes for biomass production alcoholic fermentation can be avoided by glucose-limited fed-batch cultivation and intensive aeration and mixing. However, control of specific growth rate at or below μcrit results in a lower productivity of biocatalyst production as compared to growth at μmax or at least above μcrit.

In order to construct a yeast strain in which the metabolism is diverted from alcoholic fermentation to respiration and/or the strain exhibits a higher μcrit, one could block the fermentative pathway or stimulate the respiratory pathway. The former approach includes deletion or mutation of genes encoding pyruvate decarboxylase (PDC). An alternative is to up-regulate the expression of a regulator that has a global effect on respiration. However, if high productivity for alcohol production in fermentation is desired, either under high-glucose aerobic conditions or under anaerobic conditions, expression of genes to stimulate the respiratory pathway may not be advantageous, rather deleterious for alcohol formation.

Provided herein are engineered yeast recombinant cells comprising a recombinant polynucleotide encoding a gene for a subunit of the HAP transcriptional complex and an engineered higher alcohol biosynthetic pathway. Also provided herein is a differential expression of genes under different conditions, thus providing a strategy for differential expression during biocatalyst propagation and fermentation product production phases. In embodiments, the gene is expressed during propagation phase of a fermentation-based production process, and is down-regulated or not expressed during production phase of a fermentation-based production process. In embodiments, the gene is operably linked to a conditional promoter. In embodiments, the recombinant yeast cells further comprise at least one genetic modification that reduces or eliminates activity of an endogenous pyruvate decarboxylase. Also provided herein are methods for generating a recombinant yeast cell, comprising introducing into a yeast cell (a) a recombinant polynucleotide encoding a gene for a subunit of the HAP transcriptional complex, and (b) an engineered higher alcohol biosynthetic pathway. The inventors have also provided a method for increasing maximum specific growth rate of a yeast cell, comprising introducing into a yeast cell (a) a recombinant polynucleotide encoding a gene for a subunit of the HAP transcriptional complex, and (b) an engineered higher alcohol biosynthetic pathway; wherein growth rate of the yeast cell during fermentation-based production process is greater when compared to growth rate of a yeast cell that does not contain a recombinant polynucleotide encoding a gene for a subunit of the HAP transcriptional complex. The inventors have also provided a method for increasing μcrit of a yeast cell, comprising introducing into a yeast cell (a) a recombinant polynucleotide encoding a gene for a subunit of the HAP transcriptional complex, and (b) an engineered higher alcohol biosynthetic pathway; wherein μcrit of the yeast cell during fermentation-based production process is greater when compared to μcrit a yeast cell that does not contain a recombinant polynucleotide encoding a gene for a subunit of the HAP transcriptional complex. The inventors have also provided a method for increasing biomass yield of a yeast cell, comprising introducing into a yeast cell (a) a recombinant polynucleotide encoding a gene for a subunit of the HAP transcriptional complex, and (b) an engineered higher alcohol biosynthetic pathway; wherein biomass of the yeast cell during fermentation-based production process is greater when compared to biomass of a yeast cell that does not contain a recombinant polynucleotide encoding a gene for a subunit of the HAP transcriptional complex.

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

A used herein, the terms “comprises,” “comprising,” “includes,” “including,” “has,” “having,” “contains,” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements not expressly listed or inherent to only those elements but can include other elements not expressly listed or inherent to such composition, mixture, process, method, article, or apparatus. Further, unless expressly stated to the contrary, “or” refers to an inclusive or and not to exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences of the element or component. Therefore, “a” or “an” should be read to include one or at least one, and the singular word form of the element or component also includes the plural unless the number is obviously meant to be singular.

The term “invention” as used herein is a non-limiting term and is not intended to refer to any single embodiment of the particular invention but encompasses all possible embodiments as described in the application.

The term “propagation phase” refers to the fermentation-based production process steps during which cell biomass is produced and inoculum build-up occurs.

The term “production phase” refers to the fermentation-based production process steps during which a desired fermentation product, including, but not limited to butanol, isobutanol, 1-butanol, 2-butanol and/or 2-butanone production, occurs.

The term “fermentation-based production process” refers to any process that uses living cells or their components to obtain a desired product(s). A fermentation-based production process can include, but is not limited to, propagation of the yeast to produce desired biomass concentration, fermentation of yeast to obtain desired products, and, optionally, recovery of the desired product.

In some instances, “biomass” as used herein refers to the cell biomass of the fermentation product-producing microorganism, typically provided in units g/l dry cell weight (dcw).

The term “fermentation product” includes any desired product of interest, including lower alkyl alcohols including, but not limited to butanol, lactic acid, 3-hydroxy-propionic acid, acrylic acid, acetic acid, succinic acid, citric acid, fumaric acid, malic acid, itaconic acid, 1,3-propane-diol, ethylene, glycerol, isobutyrate, etc.

A recombinant host cell comprising an “engineered higher alcohol biosynthetic pathway” (such as an engineered butanol or isobutanol biosynthetic pathway) refers to a host cell containing a modified pathway that produces alcohol in a manner different than that normally present in the host cell. Such differences include production of an alcohol not typically produced by the host cell, or increased or more efficient production.

The term “higher alcohol” refers to any straight-chain or branched, saturated or unsaturated, alcohol molecule with 4 or more carbon atoms. Higher alcohols include, but are not limited to, 1-butanol, 2-butanol, isobutanol, pentanol, or mixtures thereof.

The term “butanol” refers to 1-butanol, 2-butanol, isobutanol, or mixtures thereof. Isobutanol is also known as 2-methyl-1-propanol.

The term “butanol biosynthetic pathway” as used herein refers to an enzyme pathway to produce 1-butanol, 2-butanol, or isobutanol. For example, isobutanol biosynthetic pathways are disclosed in U.S. Pat. No. 7,851,188, which is incorporated by reference herein. Components of the pathways consist of all substrates, cofactors, byproducts, intermediates, end-products, and enzymes in the pathways.

The term “2-butanone biosynthetic pathway” as used herein refers to an enzyme pathway to produce 2-butanone.

A “recombinant yeast cell” is defined as a yeast cell that has been genetically manipulated. In embodiments, recombinant yeast cells have been genetically manipulated to express a biosynthetic production pathway, wherein the yeast cell either produces a biosynthetic product in greater quantities relative to an unmodified yeast cell or produces a biosynthetic product that is not ordinarily produced by an unmodified yeast cell.

The term “aerobic conditions” as used herein means conditions in the presence of oxygen.

The term “microaerobic conditions” as used herein means conditions with low levels of dissolved oxygen. For example, the oxygen level may be less than about 1% of air-saturation.

As used herein, the term “yield” refers to the amount of product per amount of carbon source in g/g. The yield may be exemplified for glucose as the carbon source. It is understood unless otherwise noted that yield is expressed as a percentage of the theoretical yield. In reference to a microorganism or metabolic pathway, “theoretical yield” is defined as the maximum amount of product that can be generated per total amount of substrate as dictated by the stoichiometry of the metabolic pathway used to make the product. For example, the theoretical yield for one typical conversion of glucose to isopropanol is 0.33 g/g. As such, a yield of isopropanol from glucose of 29.7 g/g would be expressed as 90% of theoretical or 90% theoretical yield. It is understood that while in the present disclosure the yield is exemplified for glucose as a carbon source, the invention can be applied to other carbon sources and the yield may vary depending on the carbon source used. One skilled in the art can calculate yields on various carbon sources.

The term “μcrit” refers to the specific growth rate above which fermentation products accumulate in the extracellular medium. Frequently exceeding “μcrit” at the specific growth rate corresponds to a transition of the metabolic regime at which the microorganism transitions from respiration to fermentation and gene expression is reprogrammed. This is achieved by repression of glucose-repressed genes and genes involved in gluconeogenesis, metabolism of alternate carbon sources, and respiration, etc.

The term “maximum specific growth rate” or “μₘₐₓ” refers to a maximal value of increased cell mass over time. Specific growth rate is expressed, for example, in grams of cells (g) per grams of cells (g) over time, by the symbol μ (mu), or in reciprocal time, such as hours (h-¹).

The terms “acetohydroxyacid synthase,” “acetolactate synthase” and “acetolactate synthetase” (abbreviated “ALS”, “AlsS”, “alsS” and/or “AHAS” herein) are used interchangeably herein to refer to an enzyme that catalyzes the conversion of pyruvate to acetolactate and CO₂. Example acetolactate synthases are known by the EC number 2.2.1.6 (Enzyme Nomenclature 1992, Academic Press, San Diego). These enzymes are available from a number of sources, including, but not limited to, Bacillus subtilis (GenBank Nos: CAB07802.1 (SEQ ID NO:12), Z99122 (SEQ ID NO:13), NCBI (National Center for Biotechnology Information) amino acid sequence, NCBI nucleotide sequence, respectively), CAB15618 (SEQ ID NO:14), Klebsiella pneumoniae (GenBank Nos: AAA25079 (SEQ ID NO:15), M73842 (SEQ ID NO:16)), and Lactococcus lactis (GenBank Nos: AAA25161 (SEQ ID NO:17), L16975 (SEQ ID NO:18)).

The term “ketol-acid reductoisomerase” (“KARI”), and “acetohydroxy acid isomeroreductase” will be used interchangeably and refer to enzymes capable of catalyzing the reaction of (S)-acetolactate to 2,3-dihydroxyisovalerate. Example KARI enzymes may be classified as EC number EC 1.1.1.86 (Enzyme Nomenclature 1992, Academic Press, San Diego), and are available from a vast array of microorganisms, including, but not limited to, Escherichia coli (GenBank Nos: NP_418222 (SEQ ID NO: 19), NC_000913 (SEQ ID NO:20)), Saccharomyces cerevisiae (GenBank Nos: NP_013459 (SEQ ID NO:21), NC_001144 (SEQ ID NO:22)), Methanococcus maripaludis (GenBank Nos: CAF30210 (SEQ ID NO:23), BX957220 (SEQ ID NO:24)), and Bacillus subtilis (GenBank Nos: CAB14789 (SEQ ID NO:25), Z99118 (SEQ ID NO:26)). KARIs include Anaerostipes caccae KARI variants “K9G9”, “K9D3”, and “K9JB4P” (SEQ ID NOs:27, 28, and 29 respectively). In some embodiments, KARI utilizes NADH. In some embodiments, KARI utilizes NADPH.

The term “acetohydroxy acid dehydratase” (“DHAD”) refers to an enzyme that catalyzes the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, E. coli (GenBank Nos: YP_026248 (SEQ ID NO:30), NC_000913 (SEQ ID NO:31)), S. cerevisiae (GenBank Nos: NP_012550 (SEQ ID NO:32), NC 001142 (SEQ ID NO:33)), M. maripaludis (GenBank Nos: CAF29874 (SEQ ID NO:34), BX957219 (SEQ ID NO:35)), B. subtilis (GenBank Nos: CAB14105 (SEQ ID NO:36), Z99115 (SEQ ID NO:37)), L. lactis, N. crassa, and S. mutans. DHADs include S. mutans variant “I2V5” (SEQ ID NO:38)

The term “branched-chain keto acid decarboxylase” refers to an enzyme that catalyzes the conversion of α-ketoisovalerate to isobutyraldehyde and CO₂. Example branched-chain keto acid decarboxylases are known by the EC number 4.1.1.72 and are available from a number of sources, including, but not limited to, Lactococcus lactis (GenBank Nos: AAS49166 (SEQ ID NO:39), AY548760 (SEQ ID NO:40); CAG34226 (SEQ ID NO:41), AJ746364 (SEQ ID NO:42), Salmonella typhimurium (GenBank Nos: NP_461346 (SEQ ID NO:43), NC_003197 (SEQ ID NO:44)), Clostridium acetobutylicum (GenBank Nos: NP_149189 (SEQ ID NO:45), NC_001988 (SEQ ID NO:46)), M. caseolyticus (SEQ ID NO:47), and L. grayi (SEQ ID NO:48).

The term “branched-chain alcohol dehydrogenase” (“ADH”) refers to an enzyme that catalyzes the conversion of isobutyraldehyde to isobutanol. Example branched-chain alcohol dehydrogenases are known by the EC number 1.1.1.265, but may also be classified under other alcohol dehydrogenases (specifically, EC 1.1.1.1 or 1.1.1.2). Alcohol dehydrogenases may be NADPH dependent or NADH dependent. Such enzymes are available from a number of sources, including, but not limited to, S. cerevisiae (GenBank Nos: NP_010656 (SEQ ID NO:49), NC_001136 (SEQ ID NO:50); NP_014051 (SEQ ID NO:51) NC_001145 (SEQ ID NO:52)), E. coli (GenBank Nos: NP_417484 (SEQ ID NO:53), NC_000913 (SEQ ID NO:54)), C. acetobutylicum (GenBank Nos: NP_349892 (SEQ ID NO:55), NC_003030 (SEQ ID NO:56); NP_349891 (SEQ ID NO:57), NC_003030 (SEQ ID NO:58)), A. xylosoxidans, and B. indica.

The term “butanol dehydrogenase” refers to a polypeptide (or polypeptides) having an enzyme activity that catalyzes the conversion of isobutyraldehyde to isobutanol or the conversion of 2-butanone and 2-butanol. Butanol dehydrogenases are a subset of a broad family of alcohol dehydrogenases. Butanol dehydrogenase may be NAD- or NADP-dependent. The NAD-dependent enzymes are known as EC 1.1.1.1 and are available, for example, from Rhodococcus ruber (GenBank Nos: CAD36475, AJ491307). The NADP dependent enzymes are known as EC 1.1.1.2 and are available, for example, from Pyrococcus furiosus (GenBank Nos: AAC25556, AF013169). Additionally, a butanol dehydrogenase is available from Escherichia coli (GenBank Nos: NP_417484, NC_000913) and a cyclohexanol dehydrogenase is available from Acinetobacter sp. (GenBank Nos: AAG10026, AF282240). The term “butanol dehydrogenase” also refers to an enzyme that catalyzes the conversion of butyraldehyde to 1-butanol, using either NADH or NADPH as cofactor. Butanol dehydrogenases are available from, for example, C. acetobutylicum (GenBank NOs: NP_149325, NC_001988; note: this enzyme possesses both aldehyde and alcohol dehydrogenase activity); NP_349891, NC_003030; and NP_349892, NC_003030) and E. coli (GenBank NOs: NP_417484, NC_000913).

The term “pyruvate decarboxylase” refers to an enzyme that catalyzes the decarboxylation of pyruvic acid to acetaldehyde and carbon dioxide. Pyruvate dehydrogenases are known by the EC number 4.1.1.1. These enzymes are found in a number of yeast, including Saccharomyces cerevisiae (GenBank Nos: CAA97575 (SEQ ID NO:59), CAA97705 (SEQ ID NO:60), CAA97091 (SEQ ID NO:61)).

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. In embodiments, the polypeptides provided herein, including, but not limited to biosynthetic pathway polypeptides, cell integrity polypeptides, propagation polypeptides, and other enzymes comprise full-length polypeptides and active fragments thereof.

The term “gene” refers to a nucleic acid fragment that is capable of being expressed as a specific protein, optionally including regulatory sequences preceding (5′ non-coding sequences) and following (3′ non-coding sequences) the coding sequence.

As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ non-translated regions, and the like, are not part of a coding region. “Suitable regulatory sequences” refer to nucleotide sequences located upstream (5′ non-coding sequences), within, or downstream (3′ non-coding sequences) of a coding sequence that influence the transcription, RNA processing or stability, or translation of the associated coding sequence. Regulatory sequences can include promoters, translation leader sequences, introns, polyadenylation recognition sequences, RNA processing sites, effector binding sites and stem-loop structures.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters.” It is further recognized that since in most cases the exact boundaries of regulatory sequences have not been completely defined, DNA fragments of different lengths may have identical promoter activity.

In certain embodiments, the polynucleotide or nucleic acid is DNA. In the case of DNA, a polynucleotide comprising a nucleic acid, which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” or “operably linked” or “coupled” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. Other transcription control elements, besides a promoter, for example enhancers, operators, repressors, and transcription termination signals, can be operably associated with the polynucleotide. Suitable promoters and other transcription control regions are disclosed herein. An “expression construct”, as used herein, comprises a promoter nucleic acid sequence operably linked to a coding region for a polypeptide and, optionally, a terminator nucleic acid sequence.

Polynucleotide and nucleic acid coding regions of the present invention may be associated with additional coding regions which encode secretory or signal peptides, which direct the secretion of a polypeptide encoded by a polynucleotide of the present invention.

As used herein, the term “transformation” refers to the transfer of a nucleic acid fragment into the genome of a host organism, resulting in genetically stable inheritance. Host organisms containing the transformed nucleic acid fragments are referred to as “transformed” organisms or a “transformant”.

The term “expression,” “expressed,” “overexpress,” “overexpression,” or “overexpress,” or “over-expression” as used herein, refers to the transcription and stable accumulation of sense (mRNA) derived from the nucleic acid fragment of the invention. Expression may also refer to translation of mRNA into a polypeptide.

The terms “plasmid,” “vector,” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence.

As a practical matter, whether any particular nucleic acid molecule or polypeptide is at least 80%, 85%, 90%, 95%, 96%, 97%, 98%, or 99% identical to a nucleotide sequence or polypeptide sequence of the present invention can be determined conventionally using known computer programs. An exemplary method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment the query and subject sequences are both DNA sequences. An RNA sequence can be compared by converting U's to T's. The result of said global sequence alignment is in percent identity. Exemplary parameters used in a FASTDB alignment of DNA sequences to calculate percent identity are: Matrix=Unitary, k-tuple=4, Mismatch Penalty=1, Joining Penalty-30, Randomization Group Length=0, Cutoff Score=1, Gap Penalty=5, Gap Size Penalty=0.05, Window Size=500 or the length of the subject nucleotide sequences, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

Polypeptides used in the invention are encoded by nucleic acid sequences that are at least about 80%, 85%, 90%, 95%, 96%, 97%, 98%, 99%, or 100% identical to the nucleotide sequences described elsewhere in the specification, including active variants, fragments or derivatives thereof.

### Promoter Nucleic Acid Sequences—“Genetic Switches”

In some embodiments, the promoter activity is sensitive to one or more physiochemical differences between propagation and production stages of a fermentation-based production process. In embodiments, the promoter activity is sensitive to the glucose concentration. In some embodiments, the promoter activity is sensitive to the source of the fermentable carbon substrate. In still a further embodiment, the promoter activity is sensitive to the concentration of butanol in fermentation medium. In still a further embodiment, the promoter activity is sensitive to the pH in the fermentation medium. In still a further embodiment, the promoter activity is sensitive to the temperature in the fermentation medium. In embodiments, the promoter activity provides for differential expression in propagation and production stages of fermentation-based production process.

### Production and Propagation

Promoter nucleic acid sequences useful in the invention include those identified using methods known in the art such as “promoter prospecting” (described and exemplified in International Publication No. WO 2013/102147 A2 which is incorporated by reference herein in its entirety) including those that comprise nucleic acid sequences which are at least about 80%, 85%, 90%, 95%, 96%, 97%, 98%, 99%, or 100% identical to the nucleotide sequences of SEQ ID NOs:62-85, including variants, fragments or derivatives thereof that confer or increase sensitivity to fermentation conditions, such as, the concentration of oxygen, butanol, isobutyraldehyde, isobutyric acid, acetic acid, or a fermentable carbon substrate in the fermentation medium. A subset of these suitable promoter nucleic acid sequences are set forth in Tables 1 and 2 below.

TABLE 1
Promoters - Upregulated in Corn Mash Production Fermentor Compared to
Propagation Tank

| Gene/ORF | | |
|---|---|---|
| Associated | Promoter | |
| with | Polynucleotide | |
| Promoter | SEQ ID NO: | Description** |
| HXK2 | 62 | Hexokinase isoenzyme 2 that catalyzes phosphorylation of glucose in |
| | | the cytosol; predominant hexokinase during growth on glucose; |
| | | functions in the nucleus to repress expression of HXK1 and GLK1 and |
| | | to induce expression of its own gene. |
| IMA1 | 63 | Major isomaltase (alpha-1,6-glucosidase) required for isomaltose |
| | | utilization; has specificity for isomaltose, palatinose, and methyl-alpha- |
| | | glucoside; member of the IMA isomaltase family |
| SLT2 | 64 | Serine/threonine MAP kinase involved in regulating the maintenance of |
| | | cell wall integrity and progression through the cell cycle; regulated by |
| | | the PKC1-mediated signaling pathway. |
| YHR210c | 65 | Putative protein of unknown function; non-essential gene; highly |
| | | expressed under anaeorbic conditions; sequence similarity to aldose 1- |
| | | epimerases such as GAL10. |
| YJL171c | 66 | GPI-anchored cell wall protein of unknown function; induced in |
| | | response to cell wall damaging agents and by mutations in genes |
| | | involved in cell wall biogenesis; sequence similarity to |
| | | YBR162C/TOS1, a covalently bound cell wall protein. |
| PUN1 | 67 | Plasma membrane protein with a role in cell wall integrity; co-localizes |
| | | with Sur7p in punctate membrane patches; null mutant displays |
| | | decreased thermotolerance; transcription induced upon cell wall damage |
| | | and metal ion stress |
| PRE8 | 68 | Alpha 2 subunit of the 20S proteasome |
| COS3 | 69 | Protein involved in salt resistance; interacts with sodium:hydrogen |
| | | antiporter Nha1p; member of the DUP380 subfamily of conserved, |
| | | often subtelomerically-encoded proteins. |
| DIA1 | 70 | Protein of unknown function, involved in invasive and pseudohyphal |
| | | growth; green fluorescent protein (GFP)-fusion protein localizes to the |
| | | cytoplasm in a punctate pattern. |
| YNR062C | 71 | Putative membrane protein of unknown function |
| PRE10 | 72 | Alpha 7 subunit of the 20S proteasome. |
| AIM45 | 73 | Putative ortholog of mammalian electron transfer flavoprotein complex |
| | | subunit ETF-alpha; interacts with frataxin, Yfh1p; null mutant displays |
| | | elevated frequency of mitochondrial genome loss; may have a role in |
| | | oxidative stress response |

TABLE 2
Promoters Strongly-Downregulated in Corn Mash Production Fermentor
Compared to Propagation Tank

| Gene/ORF | | |
|---|---|---|
| Associated | Promoter | |
| with | Polynucleotide | |
| Promoter | SEQ ID NO: | Description** |
| ZRT1 | 74 | High-affinity zinc transporter of the plasma membrane, responsible for |
| | | the majority of zinc uptake; transcription is induced under low-zinc |
| | | conditions by the Zap1p transcription factor. |
| ZRT2 | 75 | Low-affinity zinc transporter of the plasma membrane; transcription is |
| | | induced under low-zinc conditions by the Zap1p transcription factor. |
| PHO84 | 76 | High-affinity inorganic phosphate (Pi) transporter and low-affinity |
| | | manganese transporter; regulated by Pho4p and Spt7p; mutation confers |
| | | resistance to arsenate; exit from the ER during maturation requires |
| | | Pho86p. |
| PCL1 | 77 | Cyclin, interacts with cyclin-dependent kinase Pho85p; member of the |
| | | Pcl1,2-like subfamily, involved in the regulation of polarized growth |
| | | and morphogenesis and progression through the cell cycle; localizes to |
| | | sites of polarized cell growth. |
| ARG1 | 78 | Arginosuccinate synthetase, catalyzes the formation of L- |
| | | argininosuccinate from citrulline and L-aspartate in the arginine |
| | | biosynthesis pathway; potential Cdc28p substrate. |
| ZPS1 | 79 | Putative GPI-anchored protein; transcription is induced under low-zinc |
| | | conditions, as mediated by the Zap1p transcription factor, and at |
| | | alkaline pH. |
| FIT2 | 80 | Mannoprotein that is incorporated into the cell wall via a |
| | | glycosylphosphatidylinositol (GPI) anchor, involved in the retention of |
| | | siderophore-iron in the cell wall. |
| FIT3 | 81 | Mannoprotein that is incorporated into the cell wall via a |
| | | glycosylphosphatidylinositol (GPI) anchor, involved in the retention of |
| | | siderophore-iron in the cell wall. |
| FRE5 | 82 | Putative ferric reductase with similarity to Fre2p; expression induced by |
| | | low iron levels; the authentic, non-tagged protein is detected in highly |
| | | purified mitochondria in high-throughput studies. |
| CSM4 | 83 | Protein required for accurate chromosome segregation during meiosis; |
| | | involved in meiotic telomere clustering (bouquet formation) and |
| | | telomere-led rapid prophase movements. |
| SAM3 | 84 | High-affinity S-adenosylmethionine permease, required for utilization |
| | | of S-adenosylmethionine as a sulfur source; has similarity to S- |
| | | methylmethionine permease Mmp1p. |
| FDH2 | 85 | NAD(+)-dependent formate dehydrogenase, may protect cells from |
| | | exogenous formate; YPL275W and YPL276W comprise a continuous |
| | | open reading frame in some S. cerevisiae strains but not in the genomic |
| | | reference strain S288C. |

**Descriptions for Tables 1 and 2 from Saccharomyces Genome Database (www.yeastgenome.org).

In embodiments of the invention, promoter nucleic acid sequences suitable for use in the invention comprise nucleotide sequences that are at least about 80%, 85%, 90%, 95%, 96%, 97%, 98%, 99% or 100% identical to a sequence selected from the group consisting of: SEQ ID NOs:62-85 or a variant, fragment or derivative thereof.

In embodiments, promoter nucleic acid sequences suitable for use in the invention are selected from the group consisting of: SEQ ID NOs:62-85 or a variant, fragment or derivative thereof.

### Glucose

In embodiments, a distinguishing condition between the propagation and production phases is the presence of low glucose concentrations during the propagation phase and the presence of excess glucose during the production phase. Consequently “high” vs. “low” glucose concentrations could be used to express/repress biocatalyst polypeptide expression in the propagation vs. production phase.

The hexose transporter gene family in S. cerevisiae contains the sugar transporter genes HXT1 to HXT17, GAL2 and the glucose sensor genes SNF3 and RGT2. The proteins encoded by HXT1 to HXT4 and HXT6 to HXT7 are considered to be the major hexose transporters in S. cerevisiae. The expression of most of the HXT glucose transporter genes is known to depend on the glucose concentration (Ozcan, S. and M. Johnston (1999). “Function and regulation of yeast hexose transporters.” Microbiol. Mol. Biol. Rev. 63(3): 554-69). Consequently their promoters are provided herein for differential expression of genes under “high” or “low” glucose concentrations.

In embodiments, promoter nucleic acid sequences comprising sequences from the promoter region of, HXT5 (SEQ ID NO:10), HXT7 (SEQ ID NO:11) or ADH2 (SEQ ID NO:9) are employed for higher expression under glucose-limiting conditions, and lower expression under glucose-excess conditions. HXT5, HXT6 and HXT7 show also strong expression with growth on ethanol, in contrast to HXT2 (Diderich, J. A., Schepper, M., et al. (1999). “Glucose uptake kinetics and transcription of HXT genes in chemostat cultures of Saccharomyces cerevisiae.” J. Biol. Chem. 274(22): 15350-9. It has been reported that under different oxygen conditions, HXT5 and HXT6 expression showed variability (Rintala, E., M. G. Wiebe, et al. (2008). “Transcription of hexose transporters of Saccharomyces cerevisiae is affected by change in oxygen provision.” BMC Microbiol. 8: 53.), however, equipped with this disclosure, one of skill in the art is readily able to make and test such promoter constructs under conditions relevant for a desired production process. Promoter nucleic acid sequences useful in the invention comprise those provided herein and those that comprise nucleic acid sequences which are at least about 80%, 85%, 90%, 95%, 96%, 97%, 98%, 99%, or 100% identical to the nucleotide sequences of HXT5 (SEQ ID NO:10), HXT7 (SEQ ID NO:11) or ADH2 (SEQ ID NO:9), including variants, fragments or derivatives thereof that confer or increase sensitivity to the concentration of oxygen. In embodiments, the promoter nucleic acid sequence comprises at least about 80%, 85%, 90%, 95%, 96%, 97%, 98%, 99%, or 100% identity to SEQ ID NO:10 [HXT5], 11 [HXT7] or 9 [ADH2] a fragment or derivative thereof.

### Biosynthetic Pathways

Biosynthetic pathways for the production of higher alcohols of the present invention include, for example, butanol. Butanol biosynthetic pathways that may be used include those described in U.S. Pat. Nos. 7,851,188 and 7,993,889, which are incorporated herein by reference. In embodiments, the butanol biosynthetic pathway is an isobutanol biosynthetic pathway which comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by
acetohydroxy acid reductoisomerase;
c) 2,3-dihydroxyisovalerate to α-ketoisovalerate, which may be catalyzed, for example, by acetohydroxy acid dehydratase;
d) α-ketoisovalerate to isobutyraldehyde, which may be catalyzed, for example, by a branched-chain keto acid decarboxylase; and,
e) isobutyraldehyde to isobutanol, which may be catalyzed, for example, by a branched-chain alcohol dehydrogenase.

In another embodiment, the isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by ketol-acid reductoisomerase;
c) 2,3-dihydroxyisovalerate to α-ketoisovalerate, which may be catalyzed, for example, by dihydroxyacid dehydratase;
d) α-ketoisovalerate to valine, which may be catalyzed, for example, by transaminase or valine dehydrogenase;
e) valine to isobutylamine, which may be catalyzed, for example, by valine decarboxylase;
f) isobutylamine to isobutyraldehyde, which may be catalyzed by, for example, omega transaminase; and,
g) isobutyraldehyde to isobutanol, which may be catalyzed, for example, by a branched-chain alcohol dehydrogenase.

In another embodiment, the isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
d) α-ketoisovalerate to isobutyryl-CoA, which may be catalyzed, for example, by branched-chain keto acid dehydrogenase;
e) isobutyryl-CoA to isobutyraldehyde, which may be catalyzed, for example, by acelylating aldehyde dehydrogenase; and,
f) isobutyraldehyde to isobutanol, which may be catalyzed, for example, by a branched-chain alcohol dehydrogenase.

Biosynthetic pathways for the production of 1-butanol that may be used include those described in U.S. Appl. Pub. No. 2008/0182308, which is incorporated herein by reference. In one embodiment, the 1-butanol biosynthetic pathway comprises the following substrate to product conversions:
a) acetyl-CoA to acetoacetyl-CoA, which may be catalyzed, for example, by acetyl-CoA acetyl transferase;
b) acetoacetyl-CoA to 3-hydroxybutyryl-CoA, which may be catalyzed, for example, by 3-hydroxybutyryl-CoA dehydrogenase;
c) 3-hydroxybutyryl-CoA to crotonyl-CoA, which may be catalyzed, for example, by crotonase;
d) crotonyl-CoA to butyryl-CoA, which may be catalyzed, for example, by butyryl-CoA dehydrogenase;
e) butyryl-CoA to butyraldehyde, which may be catalyzed, for example, by butyraldehyde dehydrogenase; and,
f) butyraldehyde to 1-butanol, which may be catalyzed, for example, by butanol dehydrogenase.

Biosynthetic pathways for the production of 2-butanol that may be used include those described in U.S. Appl. Pub. No. 2007/0259410 and U.S. Appl. Pub. No. 2009/0155870, which are incorporated herein by reference. In one embodiment, the 2-butanol biosynthetic pathway comprises the following substrate to product conversions:
a) pyruvate to alpha-acetolactate, which may be catalyzed, for example, by acetolactate synthase;
b) alpha-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase;
e) 3-amino-2-butanol phosphate to 2-butanone, which may be catalyzed, for example, by aminobutanol phosphate phosphorylase; and,
f) 2-butanone to 2-butanol, which may be catalyzed, for example, by butanol dehydrogenase.

In another embodiment, the 2-butanol biosynthetic pathway comprises the following substrate to product conversions:
a) pyruvate to alpha-acetolactate, which may be catalyzed, for example, by acetolactate synthase;
b) alpha-acetolactate to acetoin, which may be catalyzed, for example, by acetolactate decarboxylase;
c) acetoin to 2,3-butanediol, which may be catalyzed, for example, by butanediol dehydrogenase;
d) 2,3-butanediol to 2-butanone, which may be catalyzed, for example, by dial dehydratase; and,
e) 2-butanone to 2-butanol, which may be catalyzed, for example, by butanol dehydrogenase.

Biosynthetic pathways for the production of 2-butanone that may be used include those described in U.S. Appl. Pub. No. 2007/0259410 and U.S. Appl. Pub. No. 2009/0155870, which are incorporated herein by reference. In one embodiment, the 2-butanone biosynthetic pathway comprises the following substrate to product conversions:
a) pyruvate to alpha-acetolactate, which may be catalyzed, for example, by acetolactate synthase;
b) alpha-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase; and,
e) 3-amino-2-butanol phosphate to 2-butanone, which may be catalyzed, for example, by aminobutanol phosphate phosphorylase.

In another embodiment, the 2-butanone biosynthetic pathway comprises the following substrate to product conversions:
a) pyruvate to alpha-acetolactate, which may be catalyzed, for example, by acetolactate synthase;
b) alpha-acetolactate to acetoin which may be catalyzed, for example, by acetolactate decarboxylase;
c) acetoin to 2,3-butanediol, which may be catalyzed, for example, by butanediol dehydrogenase; and,
d) 2,3-butanediol to 2-butanone, which may be catalyzed, for example, by diol dehydratase.

### Recombinant Yeast Host Cells

Standard recombinant DNA and molecular cloning techniques are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley Interscience (1987). Additional methods are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Molecular tools and techniques are known in the art and include splicing by overlapping extension polymerase chain reaction (PCR) (Yu, et al. (2004) Fungal Genet. Biol. 41:973-981), positive selection for mutations at the URA3 locus of Saccharomyces cerevisiae (Boeke, J. D. et al. (1984) Mol. Gen. Genet. 197, 345-346; M A Romanos, et al. Nucleic Acids Res. 1991 Jan. 11; 19(1): 187), the cre-lox site-specific recombination system as well as mutant lox sites and FLP substrate mutations (Sauer, B. (1987) Mol Cell Biol 7: 2087-2096; Senecoff, et al. (1988) Journal of Molecular Biology, Volume 201, Issue 2, Pages 405-421; Albert, et al. (1995) The Plant Journal. Volume 7, Issue 4, pages 649-659), “seamless” gene deletion (Akada, et al. (2006) Yeast; 23(5):399-405), and gap repair methodology (Ma et al., Genetics 58:201-216; 1981).

The genetic manipulations of a recombinant host cell disclosed herein can be performed using standard genetic techniques and screening and can be made in any host cell that is suitable to genetic manipulation (Methods in Yeast Genetics, 2005, Cold Spring Harbor Laboratory Press, Cold Spring Harbor, N.Y., pp. 201-202).

Non-limiting examples of host cells for use in the invention include filamentous fungi and yeasts. In one embodiment, the recombinant yeast cell comprises or is selected from the group consisting of: Saccharomyces cerevisiae, Schizosaccharomyces pombe, Kluyveromyces lactis, Kluyveromyces thermotolerans, Candida glabrata, Candida albicans, Pichia stipitis, or Yarrowia lipolytica.

In some embodiments, the yeast is Crabtree-positive. Crabtree-positive yeast cells demonstrate the Crabtree effect, which is a phenomenon whereby cellular respiration is inhibited when a high concentration of glucose is present in aerobic culture medium. Suitable Crabtree-positive yeast are viable in culture and include, but are not limited to, Saccharomyces, Schizosaccharomyces, and Issatchenkia. Suitable species include, but are not limited to, Saccharomyces cerevisiae, Schizosaccharomyces pombe, Kluyveromyces thermotolerans, Candida glabrata, Issatchenkia orientalis.

Crabtree-positive yeast cells may be grown with high aeration and in low glucose concentration to maximize respiration and cell mass production, as known in the art, rather than butanol production. Typically the glucose concentration is kept to less than about 0.2 g/L. The aerated culture can grow to a high cell density and then be used as the present production culture. Alternatively, yeast cells that are capable of producing butanol may be grown and concentrated to produce a high cell density culture.

In some embodiments, the yeast is Crabtree-negative. Crabtree-negative yeast cells do not demonstrate the Crabtree effect when a high concentration of glucose is added to aerobic culture medium, and therefore, in Crabtree-negative yeast cells, alcoholic fermentation is absent after an excess of glucose is added. Suitable Crabtree-negative yeast genera are viable in culture and include, but are not limited to, Hansenula, Debaryomyces, Yarrowia, Rhodotorula, and Pichia. Suitable species include, but are not limited to, Candida utilis, Hansenula nonfermentans, Kluyveromyces marxianus, Kluyveromyces lactis, Pichia stipitis, and Pichia pastoris.

Suitable microbial hosts may include, but are not limited to, members of the genera Clostridium, Zymomonas, Escherichia, Salmonella, Rhodococcus, Pseudomonas, Bacillus, Vibrio, Lactobacillus, Enterococcus, Alcaligenes, Klebsiella, Paenibacillus, Arthrobacter, Corynebacterium, Brevibacterium, Pichia, Candida, Issatchenkia, Hansenula, Kluyveromyces, and Saccharomyces. Suitable hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae. In some embodiments, the host cell is Saccharomyces cerevisiae. S. cerevisiae yeast are known in the art and are available from a variety of sources, including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

Recombinant microorganisms containing the necessary genes that will encode the enzymatic pathway for the conversion of a fermentable carbon substrate to a desired product (e.g. butanol) can be constructed using techniques well known in the art. For example, genes encoding the enzymes of one of the isobutanol biosynthetic pathways of the invention, for example, acetolactate synthase, acetohydroxy acid isomeroreductase, acetohydroxy acid dehydratase, branched-chain α-keto acid decarboxylase, and branched-chain alcohol dehydrogenase, can be obtained from various sources, as described above.

Methods of obtaining desired genes from a genome are common and well known in the art of molecular biology. For example, if the sequence of the gene is known, suitable genomic libraries can be created by restriction endonuclease digestion and can be screened with probes complementary to the desired gene sequence. Once the sequence is isolated, the DNA can be amplified using standard primer-directed amplification methods such as polymerase chain reaction (U.S. Pat. No. 4,683,202) to obtain amounts of DNA suitable for transformation using appropriate vectors. Tools for codon optimization for expression in a heterologous host are readily available (described elsewhere herein).

Once the relevant pathway genes are identified and isolated they can be transformed into suitable expression hosts by means well known in the art. Vectors or cassettes useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions can be derived from genes homologous to the transformed host cell, although it is to be understood that such control regions can also be derived from genes that are not native to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements in a given host cell, including those used in the Examples, is suitable for the present invention including, but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, 1PL, 1PR, T7, tac, and trc (useful for expression in Escherichia coli, Alcaligenes, and Pseudomonas) as well as the amy, apr, npr promoters and various phage promoters useful for expression in Bacillus subtilis, Bacillus licheniformis, and Paenibacillus macerans. For yeast recombinant host cells, a number of promoters can be used in constructing expression cassettes for genes, including, but not limited to, the following constitutive promoters suitable for use in yeast: FBA1, TDH3 (GPD), ADH1, ILV5, and GPM1; and the following inducible promoters suitable for use in yeast: GAL1, GAL10, OLE1, and CUP1. Other yeast promoters include hybrid promoters UAS(PGK1)-FBA1p, UAS(PGK1)-ENO2p, UAS(FBA1)-PDC1p, UAS(PGK1)-PDC1p, and UAS(PGK)-OLE1p.

Promoters, transcriptional terminators, and coding regions can be cloned into a yeast 2 micron plasmid and transformed into yeast cells (Ludwig et al. Gene, 132: 33-40, 1993; US Appl. Pub. No. 20080261861A1).

Adjusting the amount of gene expression in a given host may be achieved by varying the level of transcription, such as through selection of native or artificial promoters. In addition, techniques such as the use of promoter libraries to achieve desired levels of gene transcription are well known in the art. Such libraries can be generated using techniques known in the art, for example, by cloning of random cDNA fragments in front of gene cassettes (Goh et al. (2002) AEM 99, 17025), by modulating regulatory sequences present within promoters (Ligr et al. (2006) Genetics 172, 2113), or by mutagenesis of known promoter sequences (Alper et al. (2005) PNAS, 12678; Nevoigt et al. (2006) AEM 72, 5266).

Termination control regions can also be derived from various genes native to the hosts. Optionally, a termination site can be unnecessary or can be included.

Certain vectors are capable of replicating in a broad range of host bacteria and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid, 50: 74-79, 2003). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to effect gene replacement in a range of Gram-positive bacteria (Maguin et al., J. Bacteriol., 174: 5633-5638, 1992). Additionally, in vitro transposomes are available to create random mutations in a variety of genomes from commercial sources such as EPICENTRE®.

The expression of a biosynthetic pathway in various microbial hosts is described in more detail in the Examples herein and in the art. U.S. Pat. No. 7,851,188 and PCT App. No. WO2012/129555, both incorporated by reference, which disclose the engineering of recombinant microorganisms for production of isobutanol. U.S. Appl. Pub. No. 2008/0182308A1, incorporated by reference, discloses the engineering of recombinant microorganisms for production of 1-butanol. U.S. Appl. Pub. Nos. 2007/0259410A1 and 2007/0292927A1, both incorporated by reference, disclose the engineering of recombinant microorganisms for production of 2-butanol. Multiple pathways are described for biosynthesis of isobutanol and 2-butanol. The methods disclosed in these publications can be used to engineer the recombinant host cells of the present invention. The information presented in these publications is hereby incorporated by reference in its entirety.

### Modifications

In some embodiments, the host cells comprising a biosynthetic pathway as provided herein may further comprise one or more additional modifications. U.S. Appl. Pub. No. 2009/0305363 (incorporated herein by reference) discloses increased conversion of pyruvate to acetolactate by engineering yeast for expression of a cytosol-localized acetolactate synthase and substantial elimination of pyruvate decarboxylase activity. Modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Appl. Pub. No. 2009/0305363 (incorporated herein by reference), modifications to a host cell that provide for increased carbon flux through an Entner-Doudoroff Pathway or reducing equivalents balance as described in U.S. Appl. Pub. No. 2010/0120105 (incorporated herein by reference). Other modifications include integration of at least one polynucleotide encoding a polypeptide that catalyzes a step in a pyruvate-utilizing biosynthetic pathway. Other modifications are described in PCT. Pub. No. WO2012/129555, incorporated herein by reference. Modifications include at least one deletion, mutation, and/or substitution in an endogenous polynucleotide encoding a polypeptide having acetolactate reductase activity. In embodiments, the polypeptide having acetolactate reductase activity is YMR226C of Saccharomyces cerevisiae or a homolog thereof. Additional modifications include a deletion, mutation, and/or substitution in an endogenous polynucleotide encoding a polypeptide having aldehyde dehydrogenase and/or aldehyde oxidase activity. In embodiments, the polypeptide having aldehyde dehydrogenase activity is ALD6 from Saccharomyces cerevisiae or a homolog thereof. A genetic modification which has the effect of reducing glucose repression wherein the yeast production host cell is pdc− is described in U.S. Appl. Pub. No. 2011/0124060, incorporated herein by reference. In some embodiments, the pyruvate decarboxylase that is deleted or downregulated is selected from the group consisting of: PDC1, PDC5, PDC6, or combinations thereof. In some embodiments, host cells contain a deletion or downregulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

Recombinant host cells may further comprise (a) at least one heterologous polynucleotide encoding a polypeptide having dihydroxy-acid dehydratase activity; and (b)(i) at least one deletion, mutation, and/or substitution in an endogenous gene encoding a polypeptide affecting Fe—S cluster biosynthesis; and/or (ii) at least one heterologous polynucleotide encoding a polypeptide affecting Fe—S cluster biosynthesis, described in PCT Publication No. WO2011/103300, incorporated herein by reference. In embodiments, the polypeptide affecting Fe—S cluster biosynthesis is encoded by AFT1, AFT2, FRA2, GRX3, or CCC1. In embodiments, the polypeptide affecting Fe—S cluster biosynthesis is constitutive mutant AFT1 L99A, AFT1 L102A, AFT1 C291F, or AFT1 C293F.

### Differential Expression

As demonstrated in the Examples, a recombinant host cell comprising promoter nucleic acid sequences may be subjected to different conditions, such as conditions corresponding to those in propagation vs. production phase, and differential expression of a target polynucleotide or its encoded polypeptide may be confirmed using methods known in the art and/or provided herein. Differential expression of a polynucleotide encoding a biocatalyst polypeptide can be confirmed by comparing transcript levels under different conditions using reverse transcriptase polymerase chain reaction (RT-PCR) or real-time PCR using methods known in the art and/or exemplified herein. In some embodiments, a reporter, such as green fluorescent protein (GFP) can be used in combination with flow cytometry to confirm the capability of a promoter nucleic acid sequence to affect expression under different conditions. Furthermore, the activity of a biocatalyst polypeptide may be determined under different conditions to confirm the differential expression of the polypeptide using methods known in the art. For example, where ALS is the biocatalyst polypeptide, the activity of ALS present in host cells subjected to different conditions may be determined (using, for example, methods described in W. W. Westerfeld (1945), J. Biol. Chem. 161:495-502, modified as described in the Examples herein). A difference in ALS activity can be used to confirm differential expression of the ALS. It is also envisioned that differential expression of a biocatalyst polypeptide can be confirmed indirectly by measurement of downstream products or byproducts. For example, a decrease in production of isobutyraldehyde may be indicative of differential ALS expression.

It will be appreciated that other useful methods to confirm differential expression include measurement of biomass and/or measurement of biosynthetic pathway products under different conditions. For example, spectrophotometric measurement of optical density (O.D.) can be used as an indicator of biomass. Measurement of pathway products or by-products, including, but not limited to butanol concentration, DHMB concentration, or isobutyric acid can be carried out using methods known in the art and/or provided herein such as high pressure liquid chromatography (HPLC; for example, see PCT. Pub. No. WO2012/129555, incorporated herein by reference) Likewise, the rate of biomass increase, the rate of glucose consumption, or the rate of butanol production can be determined, for example by using the indicated methods. Biomass yield and product (e.g. butanol) yield can likewise be determined using methods disclosed in the art and/or herein.

### Methods for Producing Fermentation Products

Another embodiment of the present invention is directed to methods for producing various fermentation products including, but not limited to, higher alcohols. These methods employ the recombinant host cells of the invention. In one embodiment, the method of the present invention comprises providing a recombinant yeast cell as discussed above, contacting the recombinant yeast cell with a fermentable carbon substrate in a fermentation medium under conditions whereby the fermentation product is produced and, optionally, recovering the fermentation product.

It will be appreciated that a process for producing fermentation products may comprise multiple phases. For example, process may comprise a first biomass production phase, a second biomass production phase, a fermentation production phase, and an optional recovery phase. In embodiments, processes provided herein comprise more than one, more than two, or more than three phases. It will be appreciated that process conditions may vary from phase to phase. For example, one phase of a process may be substantially aerobic, while the next phase may be substantially anaerobic. Other differences between phases may include, but are not limited to, source of carbon substrate (e.g. feedstock from which the fermentable carbon is derived), carbon substrate (e.g. glucose) concentration, dissolved oxygen, pH, temperature, or concentration of fermentation product (e.g. butanol). Promoter nucleic acid sequences and nucleic acid sequences encoding biocatalyst polypeptides and recombinant host cells comprising such promoter nucleic acid sequences may be employed in such processes. In embodiments, a biocatalyst polypeptide is expressed in at least one phase.

The propagation phase generally comprises at least one process by which biomass is increased. In embodiments, the temperature of the propagation phase may be at least about 20° C., at least about 30° C., at least about 35° C., or at least about 40° C. In embodiments, the pH in the propagation phase may be at least about 4, at least about 5, at least about 5.5, at least about 6, or at least about 6.5. In embodiments, the propagation phase continues until the biomass concentration reaches at least about 5, at least about 10, at least about 15 g/L, at least about 20 g/L, at least about 30 g/L, at least about 50 g/L, at least about 70 g/L, or at least about 100 g/L. In embodiments, the average glucose or sugar concentration is about or less than about 2 g/L, about or less than about 1 g/L, about or less than about 0.5 g/L or about or less than about 0.1 g/L. In embodiments, the dissolved oxygen concentration may average as undetectable, or as at least about 10%, at least about 20%, at least about 30%, or at least about 40%.

In one non-limiting example, a stage of the propagation phase comprises contacting a recombinant yeast host cell with at least one carbon substrate at a temperature of about 30° C. to about 35° C. and a pH of about 4 to about 5.5, until the biomass concentration is in the range of about 20 g/L to about 100 g/L. The dissolved oxygen level over the course of the contact may average from about 20% to 40% (0.8-3.2 ppm). The source of the carbon substrate may be molasses or corn mash, or pure glucose or other sugar, such that the glucose or sugar concentration is from about 0 to about 1 g/L over the course of the contacting or from about 0 g/L to about 0.1 g/L. In a subsequence or alternate stage of the propagation phase, a recombinant yeast host cell may be subjected to a further process whereby recombinant yeast at a concentration of about 0.1 g/L to about 1 g/L is contacted with at least one carbon substrate at a temperature of about 25° C. to about 35° C. and a pH of about 4 to about 5.5 until the biomass concentration is in the range of about 5 g/L to about 15 g/L. The dissolved oxygen level over the course of the contact may average from undetectable to about 30% (0-2.4 ppm). The source of the carbon substrate may be corn mash such that the glucose concentration averages about 2 g/L to about 30 g/L over the course of contacting.

It will be understood that the propagation phase may comprise one, two, three, four, or more stages, and that the above non-limiting example stages may be practiced in any order or combination.

The production phase typically comprises at least one process by which a product is produced. In embodiments, the average glucose concentration during the production phase is at least about 0.1 g/L, at least about 1 g/L, at least about 5 g/L, at least about 10 g/L, at least about 30 g/L, at least about 50 g/L, or at least about 100 g/L. In embodiments, the temperature of the production phase may be at least about 20° C., at least about 30° C., at least about 35° C., or at least about 40° C. In embodiments, the pH in the production phase may be at least about 4, at least about 5, or at least about 5.5. In embodiments, the production phase continues until the product titer reaches at least about 10 g/L, at least about 15 g/L, at least about 20 g/L, at least about 25 g/L, at least about 30 g/L, at least about 35 g/L or at least about 40 g/L. In embodiments, the dissolved oxygen concentration may average as less than about 5%, less than about 1%, or as negligible such that the conditions are substantially anaerobic.

In one non-limiting example production phase, recombinant yeast cells at a concentration of about 0.1 g/L to about 6 g/L are contacted with at least one carbon substrate at a concentration of about 5 g/L to about 100 g/L, temperature of about 25° C. to about 30° C., pH of about 4 to about 5.5. The dissolved oxygen level over the course of the contact may be negligible on average, such that the contact occurs under substantially anaerobic conditions. The source of the carbon substrate may mash such as corn mash, such that the glucose concentration averages about 10 g/L to about 100 g/L over the course of the contacting, until it is substantially completely consumed.

In embodiments, the glucose concentration is about 100-fold to about 1000-fold higher in the production phase than in the propagation phase. In embodiments, the glucose concentration in production is at least about 5×, at least about 10×, at least about 50×, at least about 100×, or at least about 500× higher than that in propagation. In embodiments, the temperature in the propagation phase is about 5 to about 10 degrees lower in the production phase than in the propagation phase. In embodiments, the average dissolved oxygen concentration is anaerobic in the production phase and microaerobic to aerobic in the propagation phase.

One of skill in the art will appreciate that the conditions for propagating a host cell and/or producing a fermentation product utilizing a host cell may vary according to the host cell being used. In one embodiment, the method for producing a fermentation product is performed under anaerobic conditions. In one embodiment, the method for producing a fermentation product is performed under microaerobic conditions.

Further, it is envisioned that once a recombinant host cell comprising a suitable genetic switch has been selected, the process may be further refined to take advantage of the differential expression afforded thereby. For example, if the genetic switch provides preferential expression in high glucose conditions, one of skill in the art will be able to readily determine the glucose levels necessary to maintain minimal expression. As such, the glucose concentration in the phase of the process under which minimal expression is desired can be controlled so as to maintain minimal expression. In one non-limiting example, polymer-based slow-release feed beads (available, for example, from Kuhner Shaker, Basel, Switzerland) may be used to maintain a low glucose condition. A similar strategy can be employed to refine the propagation or production phase conditions relevant to the differential expression using the compositions and methods provided herein.

Carbon substrates may include, but are not limited to, monosaccharides (such as fructose, glucose, mannose, rhamnose, xylose or galactose), oligosaccharides (such as lactose, maltose, or sucrose), polysaccharides such as starch, maltodextrin, or cellulose, fatty acids, or mixtures thereof and unpurified mixtures from renewable feedstocks such as corn mash, cheese whey permeate, cornsteep liquor, sugar beet molasses, and barley malt. Other carbon substrates may include ethanol, lactate, succinate, or glycerol.

Additionally, the carbon substrate may also be a one carbon substrate such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth Cl Compd., [Int. Symp.], 7th (1993), 415 32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence, it is contemplated that the source of carbon utilized in the present invention may encompass a wide variety of carbon containing substrates and will only be limited by the choice of organism.

Although it is contemplated that all of the above mentioned carbon substrates and mixtures thereof may be suitable in the present invention, exemplary carbon substrates are glucose, fructose, and sucrose, or mixtures of these with C5 sugars such as xylose and/or arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in U.S. Appl. Pub. No. 2007/0031918 A1, which is herein incorporated by reference. Biomass in reference to a carbon source refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

The carbon substrates may be provided in any media that is suitable for host cell growth and reproduction. Non-limiting examples of media that can be used include M122C, MOPS, SOB, TSY, YMG, YPD, 2XYT, LB, M17, or M9 minimal media. Other examples of media that can be used include solutions containing potassium phosphate and/or sodium phosphate. Suitable media can be supplemented with NADH or NADPH.

In one embodiment, the method for producing a fermentation product results in a titer of at least about 20 g/L of a fermentation product. In another embodiment, the method for producing a fermentation product results in a titer of at least about 30 g/L of a fermentation product. In another embodiment, the method for producing a fermentation product results in a titer of at least about 10 g/L, 15 g/L, 20 g/L, 25 g/L, 30 g/L, 35 g/L or 40 g/L of fermentation product.

Non-limiting examples of lower alkyl alcohols which may be produced by the methods of the invention include butanol (for example, isobutanol), propanol, isopropanol, and ethanol. In one embodiment, isobutanol is produced.

In one embodiment, the recombinant host cell of the invention produces a fermentation product at a yield of greater than about 25%, 30%, 35%, 40%, 45%, 50%, 55%, 60%, 65%, 70%, or 75% of theoretical. In one embodiment, the recombinant host cell of the invention produces a fermentation product at a yield of greater than about 25% of theoretical. In another embodiment, the recombinant host cell of the invention produces a fermentation product at a yield of greater than about 40% of theoretical. In another embodiment, the recombinant host cell of the invention produces a fermentation product at a yield of greater than about 50% of theoretical. In another embodiment, the recombinant host cell of the invention produces a fermentation product at a yield of greater than about 75% of theoretical.

Non-limiting examples of lower alkyl alcohols produced by the recombinant host cells of the invention include butanol, isobutanol, propanol, isopropanol, and ethanol. In one embodiment, the recombinant host cells of the invention produce isobutanol. In another embodiment, the recombinant host cells of the invention do not produce ethanol.

Methods for Butanol Isolation from the Fermentation Medium

Bioproduced butanol may be isolated from the fermentation medium using methods known in the art for ABE fermentations (see, e.g., Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the butanol may be isolated from the fermentation medium using methods such as distillation, azeotropic distillation, liquid-liquid extraction, adsorption, gas stripping, membrane evaporation, or pervaporation.

Because butanol forms a low boiling point, azeotropic mixture with water, distillation can be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the butanol. In this method, the butanol containing fermentation broth is distilled to near the azeotropic composition. Then, the azeotropic mixture is condensed, and the butanol is separated from the fermentation medium by decantation. The decanted aqueous phase may be returned to the first distillation column as reflux. The butanol-rich decanted organic phase may be further purified by distillation in a second distillation column.

The butanol can also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption can also be used to isolate butanol from the fermentation medium. In this method, the fermentation broth containing the butanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the butanol from the fermentation medium. In this method, the fermentation broth containing the butanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids fatty acids, C₁₂ to C₂₂ fatty acids fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, an ester can be formed by contacting the alcohol in a fermentation medium with an organic acid (e.g., fatty acids) and a catalyst capable of esterfiying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

### Example 1

### Construction of Strains PNY1647, PNY1648, PNY1649, PNY1650, PNY1651, and PNY1652

Hap4p over-expression strains and control strains were constructed. Plasmid pBP3443 is based on the yeast centromere vector pRS413. pBP3443 (SEQ ID NO:142) was constructed to contain a chimeric gene having the coding region of the HAP4 gene from Saccharomyces cerevisiae (nt 2717-4381) expressed from the yeast FBA1 promoter (nt 2119-2708) and followed by the ADH1 terminator (nt 4390-4705) for over-expression of Hap4p. Plasmid pBP2642 (SEQ ID NO:143), also based on the yeast centromere vector pRS413, does not contain the HAP4 gene and was used for the control strain. pLH804::L2V4 (SEQ ID NO:144) was constructed to contain a chimeric gene having the coding region of the K9JB4P mutant ilvC gene from Anaeropstipes cacae (nt 1628-2659) expressed from the yeast ILV5 promoter (nt 427-1620) and followed by the ILV5 terminator (nt 2685-3307) for expression of KARI and a chimeric gene having the coding region of the L2V4 mutant ilvD gene from Streptococcus mutans (nucleotides 5356-3641) expressed from the yeast TEF1 mutant 7 promoter (nt 5766-5366; Nevoigt et al. 2006. Applied and Environmental Microbiology, v72 p5266) and followed by the FBA1 terminator (nt 3632-3320) for expression of DHAD. PNY2145 (constructed from PNY0827, deposited at the ATCC under the Budapest Treaty on Sep. 22, 2011 at the American Type Culture Collection, Patent Depository 10801 University Boulevard, Manassas, Va. 20110-2209 and has the patent deposit designation PTA-12105. Construction of PNY 2145 is described in U.S. Patent Appl. Pub. No. 2013/0252296, incorporated herein by reference) was transformed with pLH804::L2V4 and control vector pBP2642. Three transformants were selected and designated as PNY1647, PNY1648, and PNY1649 (isobutanologen control strains). PNY2145 was transformed with pLH804::L2V4 and Hap4p over-expression plasmid pBP3443. Three transformants were selected and designated PNY1650, PNY1651, and PNY1652 (isobutanologen Hap4p over-expression strains).

### Effect of Hap4p Overexpression on Growth Rate

Strains were grown to determine the effect of Hap4p overexpression on growth rate. The strains were first tested in media containing initial high glucose concentration (3%) with or without ethanol. PNY1647-PNY1652 were grown in synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 1% glucose, and with or without 0.2% ethanol. Overnight cultures were grown in 12 mL of medium in a 125 mL vented Erlenmeyer flask at 30° C., 250 RPM in a New Brunswick Scientific I24 shaker. The overnight cultures were sub-cultured into the same media, but containing 3% glucose instead of 1%, to an OD 0.02 in a final volume of 25 ml in a 250 ml vented Erlenmeyer flask and grown at 30° C., 250 RPM in a New Brunswick Scientific I24 shaker.

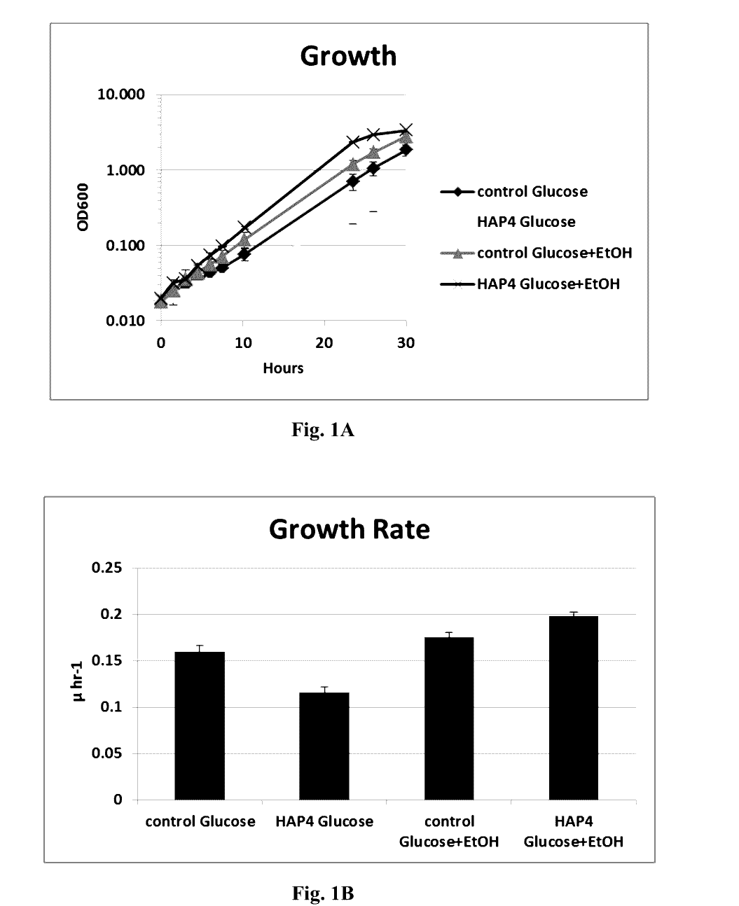

The growth rate was calculated from the growth occurring from 4.5 hours to 23.5 hours after inoculation. FIG. 1 shows that over-expression of Hap4p in the presence of the medium containing both glucose and ethanol led to an increase in the growth rate by 13%. In the medium containing only glucose, the growth rate was decreased by 28%.

### Example 2

### The Effect of Low Glucose on the Growth Rate of Yeast Strains Overexpressing Hap4 or Control Plasmid in the Presence or Absence of Ethanol

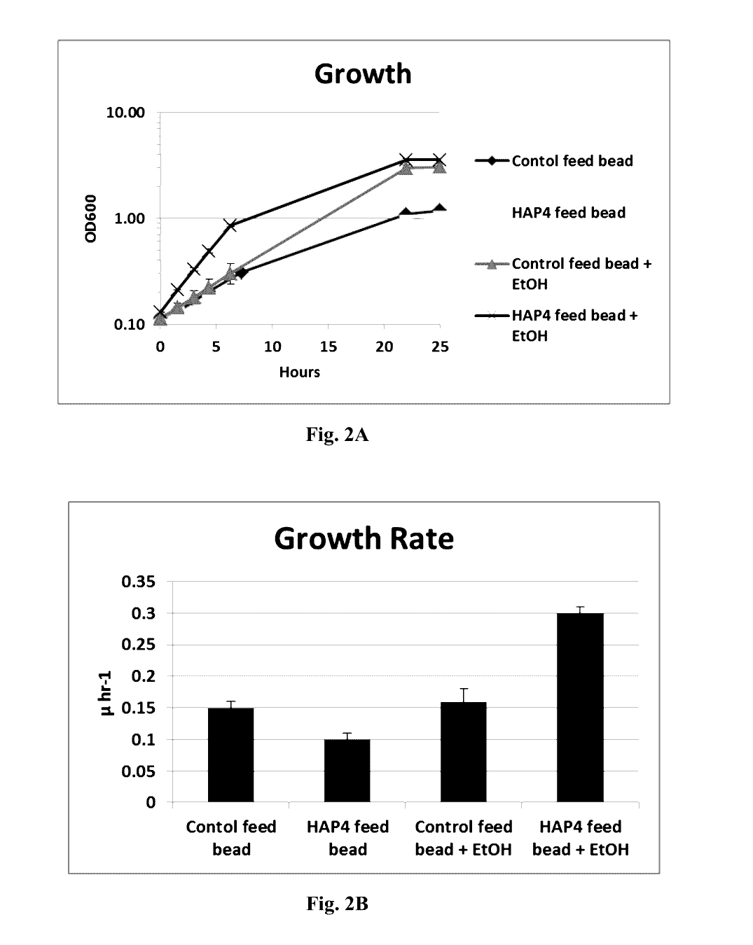

The growth of the strains was tested in a low glucose/glucose-limited condition with or without the presence of ethanol. PNY1647-PNY1652 were grown in synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, and with or without 0.1% ethanol. Strains were grown overnight in 30 ml of the above media containing one 12 mm Kuhner Shaker FeedBead Glucose disc in a 250 ml vented Erlenmeyer flask at 30° C., 250 RPM in a New Brunswick Scientific I24 shaker. The overnight cultures were sub-cultured into the same media containing one 12 mm Kuhner Shaker FeedBead Glucose disc to an OD 0.1 in a final volume of 30 ml in a 250 ml vented Erlenmeyer flask and grown at 30° C., 250 RPM in a New Brunswick Scientific I24 shaker. The growth rate was calculated from the growth occurring from approximately 1.5 hours to 7.25 hours after inoculation. FIG. 2 shows that overexpression of Hap4p in the presence of the medium containing both the glucose feed bead and ethanol led to an increase in the growth rate by 87%. In the medium containing only the glucose feed bead, the growth rate was decreased by 33%.

### Example 3

### The Growth Rate of Yeast Strains Overexpressing Hap4p or a Control Plasmid with Only Ethanol as the Carbon Source

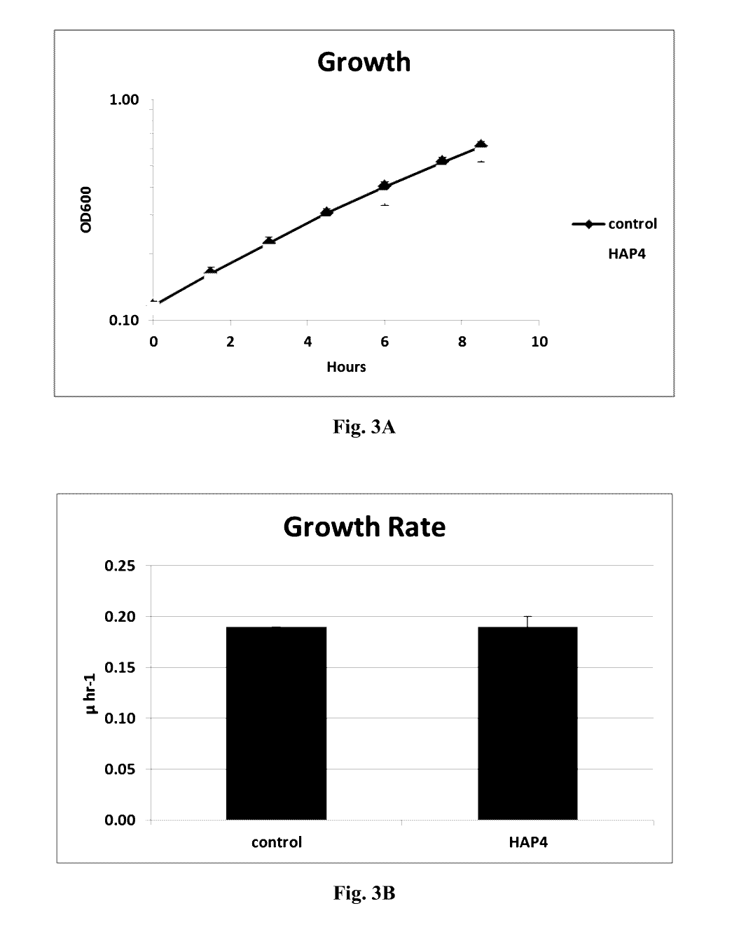

The growth of the strains was tested in media containing only ethanol as the carbon source. PNY1647-PNY1652 were grown in synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/l tryptophan, 380 mg/l leucine, 100 mM MES pH5.5, and 0.5% ethanol. Overnight cultures were grown in 10 ml of medium in a 125 ml vented Erlenmeyer flask at 30° C., 250 RPM in a New Brunswick Scientific I24 shaker. The overnight cultures were sub-cultured into the same medium to an OD 0.1 in a final volume of 20 ml in a 250 ml vented Erlenmeyer flask and grown at 30° C., 250 RPM in a New Brunswick Scientific I24 shaker. The growth rate was calculated from the growth occurring from 1.5 hours to 8.5 hours after inoculation. FIG. 3 shows that the strain with over-expression of Hap4p had the same growth rate as the control strain in the medium with only ethanol as the carbon source.

### Example 4

### The Effect of Hap4 Overexpression on Isobutanol Production

The strains were tested to determine the effect of Hap4p overexpression on isobutanol production in serum vials. PNY1647-PNY1652 were grown in synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/l tryptophan, 380 mg/l leucine, 100 mM MES pH5.5, 1% glucose, and 0.1% ethanol. Overnight cultures were grown in 10 ml of medium in a 125 ml vented Erlenmeyer flask at 30° C., 250 RPM in a New Brunswick Scientific I24 shaker. The overnight cultures were centrifuged at 4,000×g for 5 minutes at room temperature and resuspended in the above medium, but containing 3% glucose, 0.2% ethanol, and 1× vitamin mix (B6891, Sigma-Aldrich, St. Louis, Mo.). 125 mL vented Erlenmeyer flasks with the same medium (11 ml final volume) were inoculated to a final OD 600 0.07 and grown at 30° C., 250 RPM in a New Brunswick Scientific I24 shaker for 7 hours. Cultures were used to inoculate a final volume of 12 ml to an OD600 0.1 in 20 ml serum vials (Kimble Chase, Vineland, N.J.). The vials were sealed, and cultures grown at 30° C., 250 RPM in a New Brunswick Scientific I24 shaker for 42 hours.

The cultures were sampled at 0, 16, and 42 hours. Culture supernatants (collected using Spin-X centrifuge tube filter units, Costar Cat. No. 8169) were analyzed by HPLC (method described in U.S. Patent Appl. Pub. No. US 2007/0092957, incorporated by reference in its entirety) to determine the concentration of glucose and isobutanol.

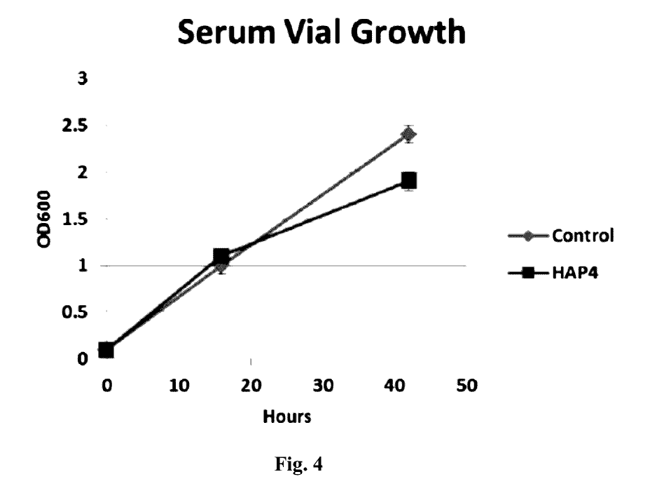

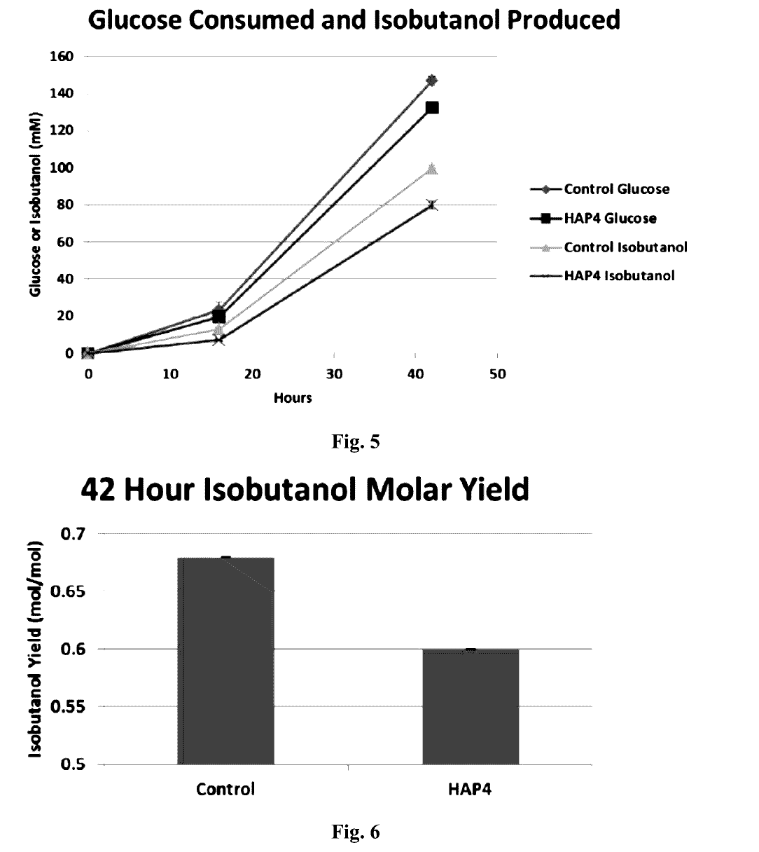

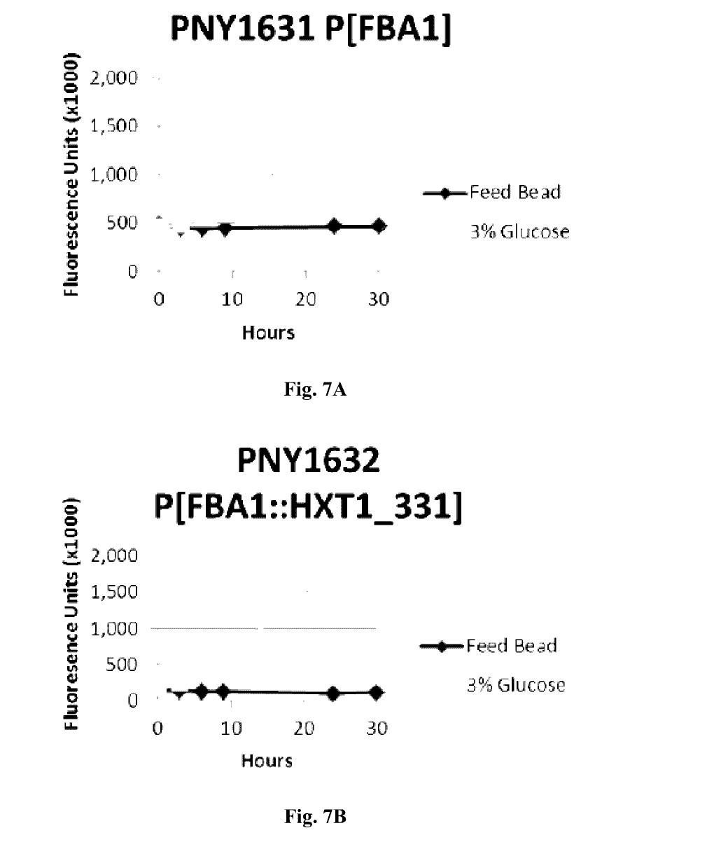

FIG. 4 shows growth of the strains in serum vials. FIG. 5 shows the amount of glucose consumed and isobutanol produced by the strains. FIG. 6 shows that the isobutanol molar yield is lower for the strains overexpressing Hap4p compared to the controls strains.

### Example 5

### Construction of Strains PNY1631, PNY1632, PNY1633, PNY1634, PNY1635, and PNY1636

Isobutanologen strains that also contain promoter-GFP (green fluorescent protein) fusions were constructed. Plasmids containing promoter-GFP fusions were based on pRS413 (ATCC#87518), a centromeric shuttle vector. The gene for the GFP protein ZsGreen (Clontech, Mountain View, Calif.) was cloned downstream of different promoters in pRS413.

Construction of PNY2115 from PNY2050

Construction of PNY2115 [MATa ura3ΔL:loxP his3Δ pdc5Δ::loxP66/71 fra2Δ 2-micron plasmid (CEN.PK2) pdc1Δ::P[PDC1]-ALS|alsS_Bs-CYClt-loxP71/66 pdc6Δ::(UAS)PGK1-P[FBA1]-KIVD|Lg(y)-TDH3t-loxP71/66 adh1Δ::P[ADH1]-ADH|Bi(y)-ADHt-loxP71/66 fra2Δ::P[ILV5]-ADH|Bi(y)-ADHt-loxP71/66 gpd2Δ::loxP71/66] from PNY2050 was as follows. PNY2050 has the genotype: MATa ura3Δ::loxP-kanMX4-loxP, his3Δ pdc1Δ::loxP71/66 pdc5Δ::loxP71/66fra2Δ 2-micron, and is described in International Publication No. WO 2013/102147 A2, which is incorporated by reference herein in its entirety.

a. pdc1Δ::P[PDC1]-ALS|alsS_Bs-CYClt-loxP71/66

To integrate alsS into the pdc1Δ::loxP66/71 locus of PNY2050 using the endogenous PDC1 promoter, an integration cassette was PCR-amplified from pLA71 (SEQ ID NO:86), which contains the gene acetolactate synthase from the species Bacillus subtilis with a FBA1 promoter and a CYC1 terminator, and a URA3 marker flanked by degenerate loxP sites to allow homologous recombination in vivo and subsequent removal of the URA3 marker. PCR was done by using the KAPA HiFi™ PCR Kit (Kapabiosystems, Woburn, Mass.) and primers 895 (SEQ ID NO:87) and 679 (SEQ ID NO:88). The PDC1 portion of each primer was derived from 60 nucleotides of the upstream of the coding sequence and 50 nucleotides that are 53 nucleotides upstream of the stop codon. The PCR product was transformed into PNY2050 using standard genetic techniques and transformants were selected on synthetic complete media lacking uracil and supplemented with 1% ethanol at 30° C. Transformants were screened to verify correct integration by colony PCR using primers 681 (SEQ ID NO:89), external to the 3′ coding region and 92 (SEQ ID NO:90), internal to the URA3 gene. Positive transformants were then prepped for genomic DNA and screened by PCR using primers N245 (SEQ ID NO:91) and N246 (SEQ ID NO:92). The URA3 marker was recycled by transforming with pLA34 (SEQ ID NO:93) containing the CRE recombinase under the GAL1 promoter and plated on synthetic complete media lacking histidine and supplemented with 1% ethanol at 30° C. Transformants were plated on rich media supplemented with 1% ethanol and 0.5% galactose to induce the recombinase. Marker removal was confirmed by patching colonies to synthetic complete media lacking uracil and supplemented with 1% ethanol to verify absence of growth. The resulting identified strain, called PNY2090 has the genotype MATa ura3ΔL:loxP, his3Δ, pdc1Δ::loxP71/66, pdc5Δ::loxP71/66 fra2Δ 2-micron pdc1Δ::P[PDC1]-ALS|alsS_Bs-CYClt-loxP71/66.

b. pdc6Δ::(UAS)PGK1-P[FBA1]-KIVD|Lg(y)-TDH3t-loxP71/66

To delete the endogenous PDC6 coding region, an integration cassette was PCR-amplified from pLA78 (SEQ ID NO:94), which contains the kivD gene from the species Listeria grayi with a hybrid FBA1 promoter and a TDH3 terminator, and a URA3 marker flanked by degenerate loxP sites to allow homologous recombination in vivo and subsequent removal of the URA3 marker. PCR was done by using the KAPA HiFi™ PCR Kit (Kapabiosystems, Woburn, Mass.) and primers 896 (SEQ ID NO:95) and 897 (SEQ ID NO:96). The PDC6 portion of each primer was derived from 60 nucleotides upstream of the coding sequence and 59 nucleotides downstream of the coding region. The PCR product was transformed into PNY2090 using standard genetic techniques and transformants were selected on synthetic complete media lacking uracil and supplemented with 1% ethanol at 30° C. Transformants were screened to verify correct integration by colony PCR using primers 365 (SEQ ID NO:97) and 366 (SEQ ID NO:98), internal primers to the PDC6 gene. Transformants with an absence of product were then screened by colony PCR N638 (SEQ ID NO:99), external to the 5′ end of the gene, and 740 (SEQ ID NO:100), internal to the FBA1 promoter. Genomic DNA was prepared from positive transformants and screened by PCR with two external primers to the PDC6 coding sequence. Positive integrants would yield a 4720 nucleotide long product, while PDC6 wild type transformants would yield a 2130 nucleotide long product. The URA3 marker was recycled by transforming with pLA34 containing the CRE recombinase under the GAL1 promoter and plated on synthetic complete media lacking histidine and supplemented with 1% ethanol at 30° C. Transformants were plated on rich media supplemented with 1% ethanol and 0.5% galactose to induce the recombinase. Marker removal was confirmed by patching colonies to synthetic complete media lacking uracil and supplemented with 1% ethanol to verify absence of growth. The resulting identified strain is called PNY2093 and has the genotype MATa ura3ΔL:loxP his3Δ pdc5Δ::loxP71/66 fra2Δ 2-micron pdc1Δ::P[PDC1]-ALS|alsS_Bs-CYClt-loxP71/66 pdc6Δ::(UAS)PGK1-P[FBA1]-KIVD|Lg(y)-TDH3t-loxP71/66.

c. adh1Δ::P[ADH1]-ADH|Bi(y)-ADHt-loxP71/66

To delete the endogenous ADH1 coding region and integrate BiADH using the endogenous ADH1 promoter, an integration cassette was PCR-amplified from pLA65 (SEQ ID NO:101), which contains the alcohol dehydrogenase from the species Beijerinckii indica with an ILV5 promoter and a ADH1 terminator, and a URA3 marker flanked by degenerate loxP sites to allow homologous recombination in vivo and subsequent removal of the URA3 marker. PCR was done by using the KAPA HiFi™ PCR Kit (Kapabiosystems, Woburn, Mass.) and primers 856 (SEQ ID NO:102) and 857 (SEQ ID NO:103). The ADH1 portion of each primer was derived from the 5′ region 50 nucleotides upstream of the ADH1 start codon and the last 50 nucleotides of the coding region. The PCR product was transformed into PNY2093 using standard genetic techniques and transformants were selected on synthetic complete media lacking uracil and supplemented with 1% ethanol at 30° C. Transformants were screened to verify correct integration by colony PCR using primers BK415 (SEQ ID NO:104), external to the 5′ coding region and N1092 (SEQ ID NO:105), internal to the BiADH gene. Positive transformants were then screened by colony PCR using primers 413 (SEQ ID NO:106), external to the 3′ coding region, and 92 (SEQ ID NO:90), internal to the URA3 marker. The URA3 marker was recycled by transforming with pLA34 (SEQ ID NO:93) containing the CRE recombinase under the GAL1 promoter and plated on synthetic complete media lacking histidine and supplemented with 1% ethanol at 30° C. Transformants were plated on rich media supplemented with 1% ethanol and 0.5% galactose to induce the recombinase. Marker removal was confirmed by patching colonies to synthetic complete media lacking uracil and supplemented with 1% ethanol to verify absence of growth. The resulting identified strain, called PNY2101 has the genotype MATa ura3ΔL:loxP his3Δ pdc5Δ::loxP71/66 fra2Δ 2-micron pdc1Δ::P[PDC1]-ALS|alsS_Bs-CYClt-loxP71/66 pdc6Δ::(UAS)PGK1-P[FBA1]-KIVD|Lg(y)-TDH3t-loxP71/66 adh1Δ::P[ADH1]-ADH|Bi(y)-ADHt-loxP71/66.

d. fra2Δ::P[ILV5]-ADH|Bi(y)-ADHt-loxP71/66

To integrate BiADH into the fra2Δ locus of PNY2101, an integration cassette was PCR-amplified from pLA65 (SEQ ID NO:101), which contains the alcohol dehydrogenase from the species Beijerinckii indica with an ILV5 promoter and an ADH1 terminator, and a URA3 marker flanked by degenerate loxP sites to allow homologous recombination in vivo and subsequent removal of the URA3 marker. PCR was done by using the KAPA HiFi™ PCR Kit (Kapabiosystems, Woburn, Mass.) and primers 906 (SEQ ID NO:107) and 907 (SEQ ID NO:108). The FRA2 portion of each primer was derived from the first 60 nucleotides of the coding sequence starting at the ATG and 56 nucleotides downstream of the stop codon. The PCR product was transformed into PNY2101 using standard genetic techniques and transformants were selected on synthetic complete media lacking uracil and supplemented with 1% ethanol at 30° C. Transformants were screened to verify correct integration by colony PCR using primers 667 (SEQ ID NO:91), external to the 5′ coding region and 749 (SEQ ID NO:109), internal to the ILV5 promoter. The URA3 marker was recycled by transforming with pLA34 (SEQ ID NO:93) containing the CRE recombinase under the GAL1 promoter and plated on synthetic complete media lacking histidine and supplemented with 1% ethanol at 30° C. Transformants were plated on rich media supplemented with 1% ethanol and 0.5% galactose to induce the recombinase. Marker removal was confirmed by patching colonies to synthetic complete media lacking uracil and supplemented with 1% ethanol to verify absence of growth. The resulting identified strain, called PNY2110 has the genotype MATa ura3Δ::loxP his3Δ pdc5Δ::loxP66/71 2-micron pdc1Δ::P[PDC1]-ALS|alsS_Bs-CYClt-loxP71/66 pdc6Δ::(UAS)PGK1-P[FBA1]-KIVD|Lg(y)-TDH3t-loxP71/66 adh1Δ::P[ADH1]-ADH|Bi(y)-ADHt-loxP71/66 fra2Δ::P[ILV5]-ADH|Bi(y)-ADHt-loxP71/66.

e. GPD2 Deletion

To delete the endogenous GPD2 coding region, a deletion cassette was PCR amplified from pLA59 (SEQ ID NO:110), which contains a URA3 marker flanked by degenerate loxP sites to allow homologous recombination in vivo and subsequent removal of the URA3 marker. PCR was done by using the KAPA HiFi™ PCR Kit (Kapabiosystems, Woburn, Mass.) and primers LA512 (SEQ ID NO:111) and LA513 (SEQ ID NO:112). The GPD2 portion of each primer was derived from the 5′ region 50 nucleotides upstream of the GPD2 start codon and 3′ region 50 nucleotides downstream of the stop codon such that integration of the URA3 cassette results in replacement of the entire GPD2 coding region. The PCR product was transformed into PNY2110 using standard genetic techniques and transformants were selected on synthetic complete medium lacking uracil and supplemented with 1% ethanol at 30° C. Transformants were screened to verify correct integration by colony PCR using primers LA516 (SEQ ID NO:113) external to the 5′ coding region and LA135 (SEQ ID NO:114), internal to URA3. Positive transformants were then screened by colony PCR using primers LA514 (SEQ ID NO:115) and LA515 (SEQ ID NO:116), internal to the GPD2 coding region. The URA3 marker was recycled by transforming with pLA34 (SEQ ID NO:93) containing the CRE recombinase under the GAL1 promoter and plated on synthetic complete medium lacking histidine and supplemented with 1% ethanol at 30° C. Transformants were plated on rich medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, called PNY2115, has the genotype MATa ura3ΔL:loxP his3Δ pdc5Δ::loxP66/71 fra2Δ 2-micron pdc1Δ::P[PDC1]-ALS|alsS_Bs-CYClt-loxP71/66 pdc6Δ::(UAS)PGK1-P[FBA1]-KIVD|Lg(y)-TDH3t-loxP71/66 adh1Δ::P[ADH1]-ADH|Bi(y)-ADHt-loxP71/66 fra2Δ::P[ILV5]-ADH|Bi(y)-ADHt-loxP71/66 gpd2Δ::loxP71/66.

pBP3836 (SEQ ID NO:117) was constructed to contain the coding region of ZsGreen (nt 2716-3411) expressed from the yeast FBA1 promoter (nt 2103-2703) and followed by the FBA1 terminator (nt 3420-4419). pBP3840 (SEQ ID NO:118) was constructed to contain the coding region of ZsGreen (nt 2891-3586) expressed from the engineered promoter FBA1::HXT1_331 (described in International Publication No. WO 2013/102147 A2, which is incorporated by reference herein in its entirety; nt 2103-2878) and followed by the FBA1 terminator (nt 3595-4594). pBP3933 (SEQ ID NO:119) was constructed to contain the coding region of ZsGreen (nt 2764-3459) expressed from the yeast ADH2 promoter (nt 2103-2751) and followed by the FBA1 terminator (nt 3468-4467). pBP3935 (SEQ ID NO:120) was constructed to contain the coding region of ZsGreen (nt 3053-3748) expressed from the yeast HXT5 promoter (nt 2103-3040) and followed by the FBA1 terminator (nt 3757-4756). pBP3937 (SEQ ID NO:121) was constructed to contain the coding region of ZsGreen (nt 3115-3810) expressed from the yeast HXT7 promoter (nt 2103-3102) and followed by the FBA1 terminator (nt 3819-4818). pBP3940 (SEQ ID NO:122) was constructed to contain the coding region of ZsGreen (nt 3065-3760) expressed from the yeast PDC1 promoter (nt 2103-3052) and followed by the FBA1 terminator (nt 3769-4768).

pLH689::I2V5 (SEQ ID NO:123) was constructed to contain a chimeric gene having the coding region of the K9JB4P variant ilvC gene from Anaeropstipes cacae (nt 1628-2659) expressed from the yeast ILV5 promoter (nt 427-1620) and followed by the ILV5 terminator (nt 2685-3307) for expression of KARI and a chimeric gene having the coding region of the I2V5 variant ilvD gene from Streptococcus mutans (nucleotides 5377-3641) expressed from the yeast TEF1 mutant 7 promoter (nt 5787-5387; Nevoigt et al. 2006. Applied and Environmental Microbiology, v72 p5266) and followed by the FBA1 terminator (nt 3632-3320) for expression of DHAD.

PNY2145 was transformed with plasmid pLH689::I2V5 and a plasmid containing one of the promoter-GFP fusions. Transformants were selected for growth on synthetic complete media lacking uracil and histidine and supplemented with 1% ethanol at 30 C. PNY2145 was transformed with plasmids pLH689::I2V5 and pBP3836 and a transformant was designated PNY1631. PNY2145 was transformed with plasmids pLH689::I2V5 and pBP3840 and a transformant was designated PNY1632. PNY2145 was transformed with plasmids pLH689::I2V5 and pBP3933 and a transformant was designated PNY1633. PNY2145 was transformed with plasmids pLH689::I2V5 and pBP3935 and a transformant was designated PNY1634. PNY2145 was transformed with plasmids pLH689::I2V5 and pBP3935 and a transformant was designated PNY1635. PNY2145 was transformed with plasmids pLH689::I2V5 and pBP3940 and a transformant was designated PNY1636.

### Example 6

### Effect of Glucose on Promoter-GFP Fusions in PNY1631, PNY1632, PNY1633, PNY1634, PNY1635, and PNY1636

This example demonstrates the response of selected promoters in isobutanologen strains to the addition of glucose to 3% (final concentration) after cells had been growing under low glucose conditions. Polymer-based slow-release feed beads (Kuhner Shaker, Basel, Switzerland) were used for the low glucose condition.

PNY1631, PNY1632, PNY1633, PNY1634, PNY1635, and PNY1636 were first grown in synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/l leucine, 100 mM MES pH5.5, and 0.5% ethanol. Overnight cultures were grown in 20 ml of medium in a 125 ml vented Erlenmeyer flask at 30° C., 250 RPM in a New Brunswick Scientific 124 shaker. The overnight cultures were centrifuged at 4,000×g for 5 minutes at room temperature and resuspended in the above medium without ethanol. Duplicate 250 ml vented flasks containing the above medium without ethanol were inoculated to an OD600 0.05 in a final volume of 35 ml for each strain. One 12 mm Kuhner Shaker FeedBead Glucose disc was added to each flask and the cultures were grown at 30° C., 250 RPM in a New Brunswick Scientific I24 shaker for 23 hours. After the 23 hours, glucose was added to one of the duplicate flasks for each strain to a final concentration of 3%, while the other duplicate flask was maintained. Growth was continued for 30 hours at 30° C., 250 RPM in a New Brunswick Scientific I24 shaker. Samples were taken prior to the addition of glucose and periodically throughout the 30 hour time period to measure OD600 and monitor promoter activity, as measured by the amount of fluorescence, using a flow cytometer.

Fluorescence was measured on a C6 Flow Cytometer (Accuri Cytometers, Inc., Ann Arbor, Mich.). Fluorescence was measured on the FL1 channel with excitation at a wavelength of 488 nm and emission detection at a wave length of 530 nm. The flow cytometer was set to measure 10,000 events at the medium flow rate (35 μl/min). Prior to loading samples on the flow cytometer, they were diluted in medium to an approximate OD600 0.1 to keep the rate of events lower than 1000 per second to ensure single cell counting.

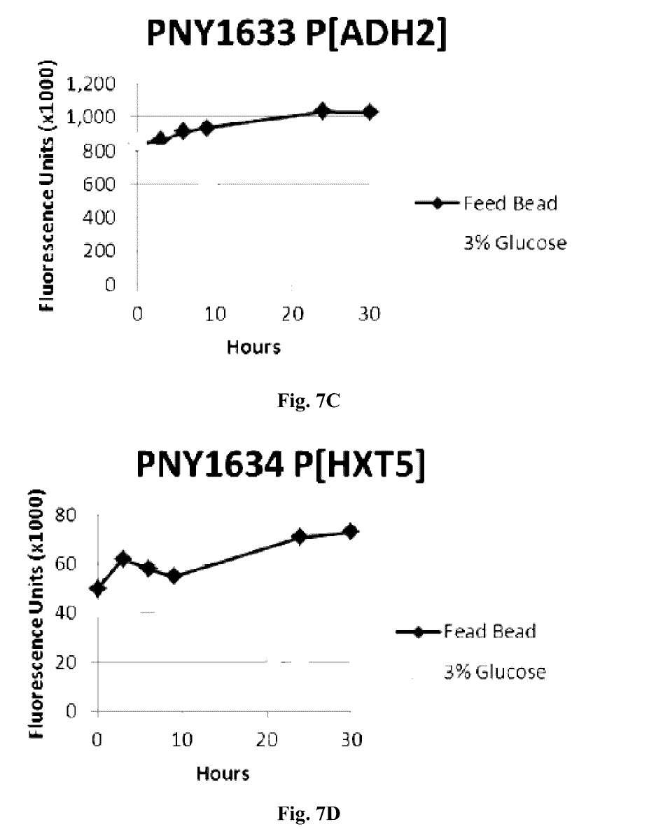

Table 3 shows the cell growth for the strains at time 0 and time 30 hours for the cultures with or without the addition of glucose to 3%. FIG. 7 shows the mean fluorescence for the 10,000 events measured at each time point for each strain with or without the addition of glucose to 3% (FIG. 7A=PNY1631, FIG. 7B=PNY1632, FIG. 7C=PNY1633, FIG. 7D=PNY1634, FIG. 7E=PNY1635, and FIG. 7F=PNY1636). PNY1632, the isobutanologen strain containing a promoter GFP fusion with the FBA1::HXT1_331 promoter engineered to be regulated by glucose in a similar fashion as the native low affinity HXT1 promoter, had fluorescence levels increase up to 11.2-fold after the addition of glucose. PNY1631, with the FBA1 promoter-GFP fusion, displayed a 3.2-fold increase in the mean fluorescence, while the PDC1 promoter-GFP strain, PNY1636, had fluorescence levels increase by only 38%. The three isobutanologen strains PNY1633, PNY1634, and PNY1635 containing the promoter-GFP fusions with the glucose repressed promoters ADH2, HXT5, and HXT7 had decreases in mean fluorescence of 4.5-fold, 2.6-fold, and 6-fold, respectively, after the addition of glucose to 3%.

TABLE 3
OD600 of cultures of strains with or
without the addition of glucose to 3%.

| | | | 0 hr 3% | 30 hr 3% |
|---|---|---|---|---|
| | 0 hr No | | glucose | glucose |
| | glucose | 30 hr No glucose | addition | addition |
| Strain | addition culture | addition culture | culture | culture |
| PNY1631 | 0.31 | 0.75 | 0.23 | 2.60 |
| PNY1632 | 0.33 | 1.00 | 0.25 | 3.22 |
| PNY1633 | 0.46 | 1.02 | 0.24 | 2.68 |
| PNY1634 | 0.29 | 0.86 | 0.24 | 2.37 |
| PNY1635 | 0.36 | 0.85 | 0.24 | 2.54 |
| PNY1636 | 0.32 | 0.80 | 0.24 | 2.40 |

### Example 7

Strains with the regulated over-expression of Hap4p were constructed. Plasmids pBP4022 and pBP4026 are based on the yeast centromere vector pRS413. pBP4022 (SEQ ID NO:145) was constructed to contain a chimeric gene having the coding region of the HAP4 gene from Saccharomyces cerevisiae (nt 2776-4440) expressed from the yeast ADH2 promoter (nt 2119-2767) and followed by the ADH1 terminator (nt 4449-4764) for the regulated over-expression of Hap4p. pBP4026 (SEQ ID NO:146) was constructed to contain a chimeric gene having the coding region of the HAP4 gene from Saccharomyces cerevisiae (nt 3127-4791) expressed from the yeast HXT7 promoter (nt 2119-3118) and followed by the ADH1 terminator (nt 4800-5115) for the regulated over-expression of Hap4p. PNY2145 was transformed with pLH804::L2V4 and pBP4022 to create strains PNY1653 and PNY1654. PNY2145 was transformed with pLH804::L2V4 and pBP4026 to create strains PNY1655 and PNY1656.

### Example 8

### Expression of HAP4 Under High Glucose Conditions with and without Ethanol

Expression levels of the HAP4 transcript were determined in strains PNY1648, PNY1649, PNY1650, and PNY1651, during growth under high glucose (3% initial concentration) conditions in the presence or absence of ethanol (0.2% initial concentration). Expression levels for the PDA1, MDH1, CYC1, and NDE1 transcripts were also determined.

The strains were grown for 24 hours in 30 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, and 1% glucose, with or without 0.2% ethanol, in 250 mL vented Erlenmeyer flasks at 30° C., 250 RPM in a New Brunswick Scientific I24 shaker. The cultures were centrifuged at 4,000×g for 5 minutes at 22° C. and resuspended in the same media, but with 3% glucose. A final volume of 30 ml of the 3% glucose media (with and without 0.2% ethanol) was inoculated with culture in 250 mL vented Erlenmeyer flasks to an OD600 0.4 and grown at 30° C., 250 RPM in a New Brunswick Scientific I24 shaker for 7 hours. Cells were harvested at 6 and 7 hours to extract RNA. 7 ml of culture was added to an ice cold 15 ml conical tube and was centrifuged at 4,000×g for 4 minutes at 4° C. Pellets were immediately resuspended in 1 ml of Trizol (Invitrogen, Carlsbad, Calif.), frozen on dry ice and then stored at −80° C. until RNA extraction.

For RNA extraction, samples were thawed on ice and transferred to 2 ml screw cap tubes containing Lysing Matrix B 0.1 mm silica spheres (MP Biomedicals, Solon, Ohio). The samples were subjected to a bead beater two times at maximum speed for one minute. 200 μl of chloroform was added and samples vortexed. The samples were centrifuged at 13,000×g for 15 minutes at 4° C. 600 μl of aqueous phase was added to 650 μl of 70% ethanol and mixed. The sample was applied to Qiagen RNeasy Kit (Qiagen, Valencia, Calif.) spin columns and the manufacturer's protocol was followed. RNA was eluted from the column with 50 μl RNase-free water. RNA samples were stored at −80° C. until real-time reverse transcription PCR analysis.

Primer Design and Validation:

Prior to expression analysis, real time PCR primers and probes were designed using Primer Express v.2.0 software from ABI/Life Technologies under default conditions. Primers were purchased from Sigma-Genosys, Woodlands, Tex., 77380. Primers were validated for specificity using BLAST analysis and for quantitation by analyzing PCR efficiency across a dilution series of target DNA. Primer efficiencies were validated with efficiencies from 90%-110%. Primer sequences are shown in Table 4 below.

TABLE 4
Primers used for RT-qPCR analysis

| | | | | SEQ ID |
|---|---|---|---|---|
| Target | Primer Name | Direction | Sequence (5′ to 3′) | NO: |
| HAP4 | HAP4-32F | for | CCGCTAGTCGCCCTCGTA | 124 |
| | HAP4-157R | rev | TGCCATCGTTTTCGAATTCC | 125 |
| | HAP4-89T | probe | 6FAM-CGCCTGTACCGATCGCCCCA-TAMRA | 126 |
| CYC1 | CYC1-64F | for | CAATGCCACACCGTGGAA | 127 |
| | CYC1-130R | rev | TGCCAAAGATACCATGCAAGTT | 128 |
| | CYC1-83T | probe | 6FAM-AGGGTGGCCCACATAAGGTTGGTCC-TAMRA | 129 |
| PDA1 | PDA1-11F | for | CTTCATTCAAACGCCAACCA | 130 |
| | PDA1-75R | rev | GGTGGGAGTGCGAAGAACA | 131 |
| | PDA1-32T | probe | 6FAM-CACAATTGGTCCGCGGGTTAGGAG-TAMRA | 132 |
| MDH1 | MDH1-329F | for | CCATCAACGCAAGCATCGT | 133 |
| | MDH1-391R | rev | CAGCATTGGGAGCGGATT | 134 |
| | MDH1-351T | probe | 6FAM-CGATTTGGCAGCAGCAACCGC-TAMRA | 135 |
| NDE1 | NDE1-1263F | for | TGCTATCGGCGATTGTACCTT | 136 |
| | NDE1-1329R | rev | ACCTTCTTGGTGGGCAACTTG | 137 |
| | NDE1-1288T | probe | 6FAM-CCTGGCTTGTTCCCTACCGC-TAMRA | 138 |
| 18S | 18S-396F | for | AGAAACGGCTACCACATCCAA | 139 |
| rRNA | 18S-468R | rev | TCACTACCTCCCTGAATTAGGATTG | 140 |
| | 18S-420T | probe | 6FAM-AAGGCAGCAGGCGCGCAAATT-TAMRA | 141 |

Real Time Reverse Transcription PCR:

2 ug of purified total RNA was treated with DNase (Qiagen PN79254) for 15 min at room temperature followed by inactivation for 5 min at 75 C in the presence of 0.1 mM EDTA. A two-step RT-PCR was then performed using 1 ug of treated RNA. In the first step RNA was converted to cDNA using the High Capacity cDNA Reverse Transcription Kit from ABI/Life Technologies (PN 4368813) according to the manufacturer's recommended protocol. The second step in the procedure was the qPCR or Real Time PCR. This was carried out on an ABI 7900HT SDS instrument. Each 20 ul qPCR reaction contained 1 ng cDNA, 0.2 ul of 100 uM forward and reverse primers, 0.05 ul TaqMan probe, 10 ul TaqMan Universal PCR Master Mix (AppliedBiosystems PN 4326614) and 8.55 ul of water. Reactions were thermal cycled while fluorescence data was collected as follows: 10 min. at 95 C followed by 40 cycles of 95 C for 15 sec and 60 C for 1 minute. A (−) reverse transcriptase RNA control of each sample was run with the 18S rRNA primer set to confirm the absence of genomic DNA. All reactions were run in triplicate.

Relative Expression Calculations:

The relative quantitation of the target genes in the samples was calculated using the ΔΔCt method (see ABI User Bulletin #2 “Relative Quantitation of Gene Expression”). 18S rRNA was used to normalize the quantitation of the target gene for differences in the amount of total RNA added to each reaction. The relative quantitation (RQ) value is the fold difference in expression of the target genes in each sample relative to the calibrator sample which has an expression level of 1.0.

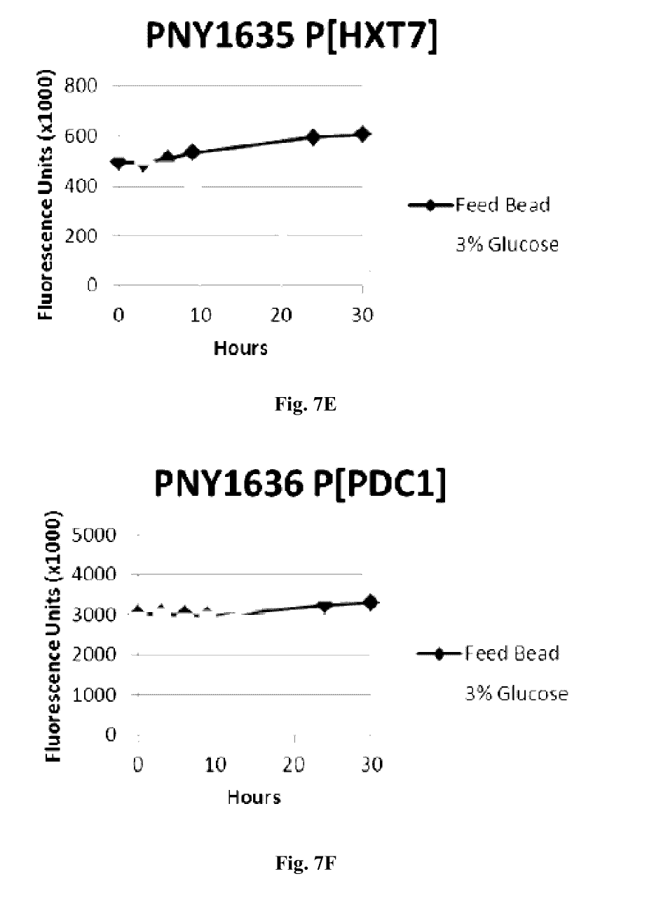

The amount of transcript present in the 6 hour time point for PNY1648 grown in the absence of ethanol was set at 1.0. The expression data in FIG. 8 are the average (both time points and strains averaged) and standard deviation for each strain type grown in either the presence or absence of ethanol. The figure demonstrates the overexpression of HAP4 mRNA in PNY1650/PNY1651 (HAP4) compared to the controls PNY1648/PNY1649 (control). Expression levels of the CYC1 mRNA, previously shown to be regulated by Hap4p (Forsburg and Guarente, 1989, Genes & Development 3:1166), followed the changes in the expression level of HAP4.

### Example 9

### Expression of HAP4 Under Low Glucose and High Glucose Conditions

Expression of the HAP4 transcript was determined in strains PNY1648, PNY1649, PNY1650, PNY1651, PNY1653, and PNY1654 during growth under low glucose and high glucose (3% initial concentration) conditions. Polymer-based slow-release feed beads (Kuhner Shaker, Basel, Switzerland) were used for the low glucose condition.

For the low glucose condition, the strains were grown overnight in 30 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 0.1% ethanol, and one 12 mm Kuhner Shaker FeedBead Glucose disc in 250 mL vented Erlenmeyer flasks at 30° C., 250 RPM in a New Brunswick Scientific I24 shaker. The overnight cultures were sub-cultured into 30 ml of the same medium (with one 12 mm Kuhner Shaker FeedBead Glucose disc) to an OD600 0.1 in 250 mL vented Erlenmeyer flasks and were grown at 30° C., 250 RPM in a New Brunswick Scientific I24 shaker to an approximate OD600 0.4. Cells were harvested to extract RNA. 10 ml of culture was added to an ice cold 15 ml conical tube and was centrifuged at 4,000×g for 4 minutes at 4° C. Pellets were immediately resuspended in 1 ml of Trizol (Invitrogen, Carlsbad, Calif.), frozen on dry ice and then stored at −80° C. until RNA extraction.

For the high glucose condition, the strains were grown overnight in 12 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 0.2% ethanol, and 1% glucose in 125 mL vented Erlenmeyer flasks at 30° C., 250 RPM in a New Brunswick Scientific I24 shaker. The overnight cultures were centrifuged at 4,000×g for 5 minutes at 22° C. and resuspended in the same medium, but with 3% glucose. A final volume of 14 ml of the 3% glucose medium was inoculated with culture in 125 mL vented Erlenmeyer flasks to an OD600 0.1 and were grown at 30° C., 250 RPM in a New Brunswick Scientific I24 shaker to an approximate OD600 0.4. Cells were harvested to extract RNA. 10 ml of culture was added to an ice cold 15 ml conical tube and was centrifuged at 4,000×g for 4 minutes at 4° C. Pellets were immediately resuspended in 1 ml of Trizol (Invitrogen, Carlsbad, Calif.), frozen on dry ice and then stored at −80° C. until RNA extraction.

For RNA extraction, samples were thawed on ice and transferred to 2 ml screw cap tubes containing Lysing Matrix B 0.1 mm silica spheres (MP Biomedicals, Solon, Ohio). The samples were subjected to a bead beater two times at maximum speed for one minute. 200 μl of chloroform was added and samples vortexed. The samples were centrifuged at 13,000×g for 15 minutes at 4° C. 600 μl of aqueous phase was added to 650 μl of 70% ethanol and mixed. The sample was applied to Qiagen RNeasy Kit (Qiagen, Valencia, Calif.) spin columns and the manufacturer's protocol was followed. RNA was eluted from the column with 50 μl RNase-free water. RNA samples were stored at −80° C. until real-time RT-PCR analysis.

Primer Design and Validation:

Prior to expression analysis, real time PCR primers and probes were designed using Primer Express v.2.0 software from ABI/Life Technologies under default conditions. Primers were purchased from Sigma-Genosys, Woodlands, Tex., 77380. Primers were validated for specificity using BLAST analysis and for quantitation by analyzing PCR efficiency across a dilution series of target DNA. Primer efficiencies were validated with efficiencies from 90%-110%. Primer sequences are shown in Table 5 below.

TABLE 5
Primers used for measuring relative mRNA expression

| | | | | SEQ |
|---|---|---|---|---|
| | | | | ID |
| Target | Primer Name | Direction | Sequence (5′ to 3′) | NO |
| HAP4 | HAP4-32F | for | CCGCTAGTCGCCCTCGTA | 124 |
| | HAP4-157R | rev | TGCCATCGTTTTCGAATTCC | 125 |
| | HAP4-89T | probe | 6FAM-CGCCTGTACCGATCGCCCCA-TAMRA | 126 |
| CYC1 | CYC1-64F | for | CAATGCCACACCGTGGAA | 127 |
| | CYC1-130R | rev | TGCCAAAGATACCATGCAAGTT | 128 |
| | CYC1-83T | probe | 6FAM-AGGGTGGCCCACATAAGGTTGGTCC-TAMRA | 129 |
| 18S | 18S-396F | for | AGAAACGGCTACCACATCCAA | 139 |
| rRNA | 18S-468R | rev | TCACTACCTCCCTGAATTAGGATTG | 140 |
| | 18S-420T | probe | 6FAM-AAGGCAGCAGGCGCGCAAATT-TAMRA | 141 |

Real Time Reverse Transcription PCR:

2 ug of purified total RNA was treated with DNase (Qiagen PN79254) for 15 min at room temperature followed by inactivation for 5 min at 75 C in the presence of 0.1 mM EDTA. A two-step RT-PCR was then performed using 1 ug of treated RNA. In the first step RNA was converted to cDNA using the High Capacity cDNA Reverse Transcription Kit from ABI/Life Technologies (PN 4368813) according to the manufacturer's recommended protocol. The second step in the procedure was the qPCR or Real Time PCR. This was carried out on an ABI 7900HT SDS instrument. Each 20 ul qPCR reaction contained 1 ng cDNA, 0.2 ul of 100 uM forward and reverse primers, 0.05 ul TaqMan probe, 10 ul TaqMan Universal PCR Master Mix (AppliedBiosystems PN 4326614) and 8.55 ul of water. Reactions were thermal cycled while fluorescence data was collected as follows: 10 min. at 95° C. followed by 40 cycles of 95° C. for 15 sec and 60° C. for 1 minute. A reverse transcriptase RNA control of each sample was run with the 18S rRNA primer set to confirm the absence of genomic DNA. All reactions were run in triplicate.

Relative Expression Calculations:

The relative quantitation of the target genes in the samples was calculated using the ΔΔCt method (see ABI User Bulletin #2 “Relative Quantitation of Gene Expression”). 18S rRNA was used to normalize the quantitation of the target gene for differences in the amount of total RNA added to each reaction. The relative quantitation (RQ) value is the fold difference in expression of the target genes in each sample relative to the calibrator sample which has an expression level of 1.0.

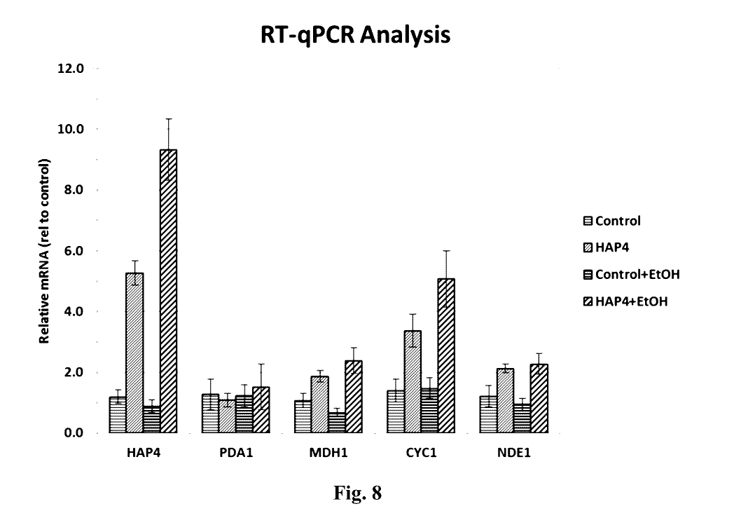

The amount of HAP4 transcript from the PNY1648 high glucose culture was set at 1.0. The expression data in FIG. 9 are the average and standard deviation for each strain type grown under each glucose condition. The figure demonstrates the regulated overexpression of HAP4 mRNA with the ADH2 promoter; higher expression under the low glucose condition. Expression levels of the CYC1 mRNA, previously shown to be regulated by Hap4p (Forsburg and Guarente, 1989, Genes & Development 3:1166), followed the changes in the expression level of HAP4.

### Example 10

### Effect of Regulated HAP4 Expression on Growth of the Isobutanologens

The growth rate of strains PNY1647, PNY1648, PNY1649, PNY1650, PNY1651, PNY1652, PNY1653, PNY1654, PNY1655, and PNY1656 was measured first under low glucose conditions, followed by high glucose (3% initial concentration) conditions, all in the presence of ethanol. Polymer-based slow-release feed beads (Kuhner Shaker, Basel, Switzerland) were used for the low glucose condition.

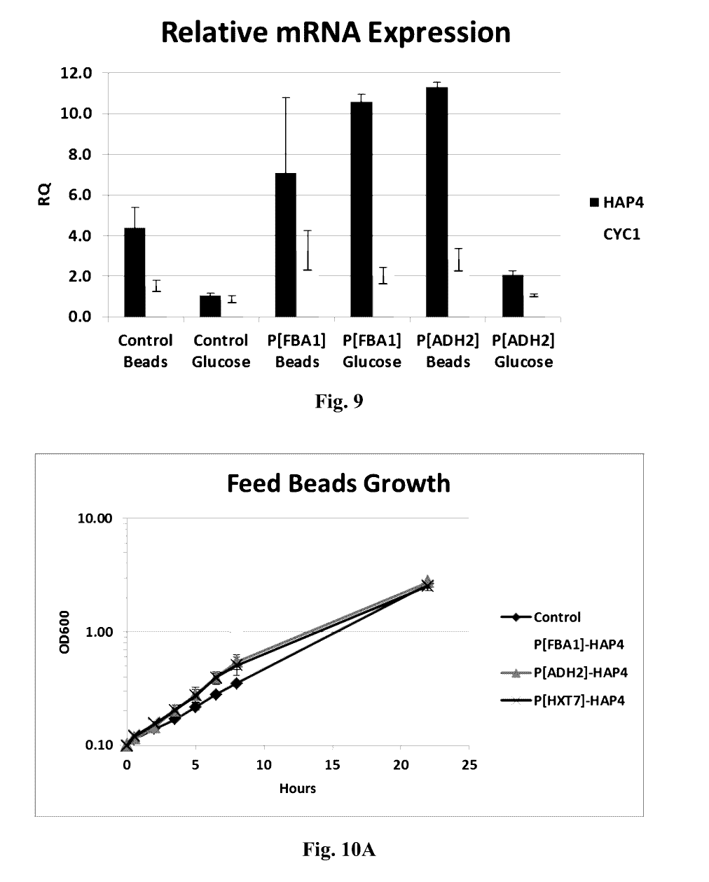

The strains were grown overnight in 30 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 0.1% ethanol, and one 12 mm Kuhner Shaker FeedBead Glucose disc in 250 mL vented Erlenmeyer flasks at 30° C., 250 RPM in a New Brunswick Scientific I24 shaker. The overnight cultures were sub-cultured into 30 ml of the same medium (with one 12 mm Kuhner Shaker FeedBead Glucose disc) to an OD600 0.1 in 250 mL vented Erlenmeyer flasks and were grown at 30° C., 250 RPM in a New Brunswick Scientific I24 shaker for 22 hours. The growth rate of the cultures was calculated during the period of growth between 2 and 8 hours. The cultures were then used to inoculate 14 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 0.2% ethanol, and 3% glucose to and OD600 0.1 in 250 mL vented Erlenmeyer flasks at and grown at 30° C., 250 RPM in a New Brunswick Scientific I24 shaker for 24.75 hours. The growth rate of the cultures was calculated during the period of growth between 3 and 9 hours. The cultures were then used to inoculate 13 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 0.2% ethanol, and 3% glucose to and OD600 0.1 in 250 mL vented Erlenmeyer flasks at and grown at 30° C., 250 RPM in a New Brunswick Scientific I24 shaker for 22.75 hours. The growth rate of the cultures was calculated during the period of growth between 5.75 and 22.75 hours. The average growth rate and standard deviation for each strain type for each growth curve is shown in FIG. 10. The figure shows the improvement in growth rate under the low glucose condition for the strains with the overexpression of HAP4 with the ADH2 and HXT7 promoters.

The growth rate of strains PNY1648, PNY1649, PNY1650, PNY1651, PNY1653, and PNY1654 was measured under low glucose conditions in the presence of acetate. Polymer-based slow-release feed beads (Kuhner Shaker, Basel, Switzerland) were used for the low glucose condition.

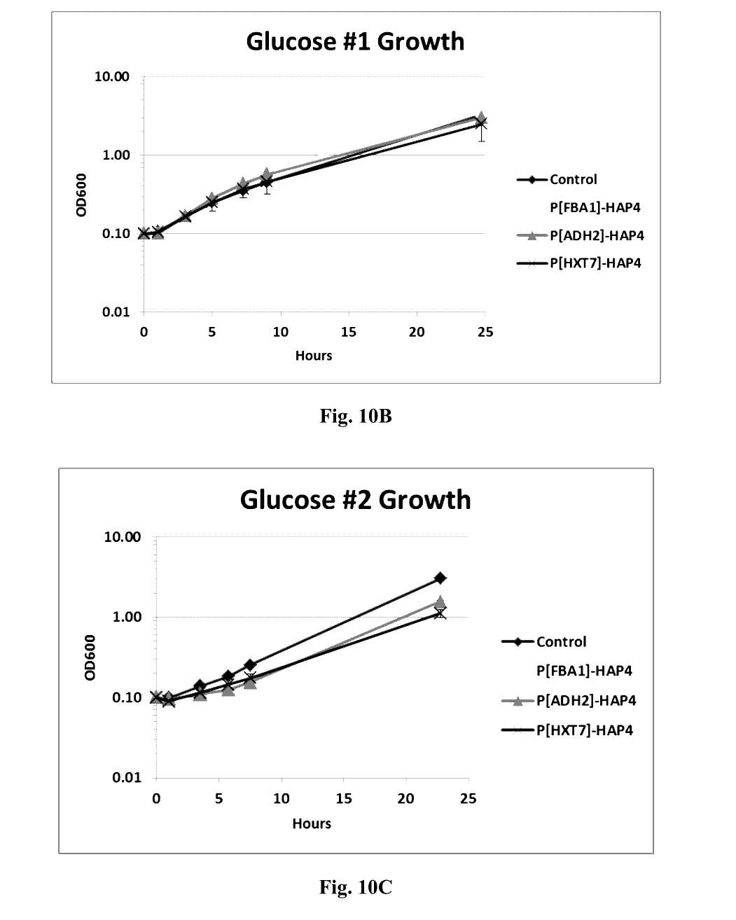

The strains were grown overnight in 30 ml of synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/L tryptophan, 380 mg/L leucine, 100 mM MES pH5.5, 0.2% sodium acetate, and one 12 mm Kuhner Shaker FeedBead Glucose disc in 250 mL vented Erlenmeyer flasks at 30° C., 250 RPM in a New Brunswick Scientific I24 shaker. The overnight cultures were sub-cultured into 30 ml of the same medium (with one 12 mm Kuhner Shaker FeedBead Glucose disc) to an OD600 0.05 in 250 mL vented Erlenmeyer flasks and were grown at 30° C., 250 RPM in a New Brunswick Scientific I24 shaker for 21 hours. The growth rate of the cultures was calculated during the period of growth between 1.5 and 7.5 hours. The average growth rate and standard deviation for each strain type is shown in FIG. 11. Like when the cultures were supplemented with ethanol, growth rate was improved for the HAP4 overexpression strains under the low glucose conditions when the medium was supplemented with sodium acetate.

### Example 11

### Effect of Regulated HAP4 Expression on Isobutanol Production

PNY1648, PNY1650, PNY1651, PNY1653, and PNY1654 were tested to determine the effect of regulated Hap4p over-expression on isobutanol production in serum vials. The strains were grown in synthetic medium (Yeast Nitrogen Base without Amino Acids (Sigma-Aldrich, St. Louis, Mo.) and Yeast Synthetic Drop-Out Media Supplement without uracil, histidine, leucine, and tryptophan (Sigma-Aldrich, St. Louis, Mo.)) supplemented with 76 mg/l tryptophan, 380 mg/l leucine, 100 mM MES pH5.5, 1% glucose, and 0.1% ethanol. Overnight cultures were grown in 10 mL of medium in a 125 ml vented Erlenmeyer flask at 30° C., 250 RPM in a New Brunswick Scientific I24 shaker. The overnight cultures were centrifuged at 4,000×g for 5 minutes at room temperature and resuspended in the above medium, but containing 3% glucose, 0.2% ethanol, and 1× vitamin mix (B6891, Sigma-Aldrich, St. Louis, Mo.). 125 ml vented Erlenmeyer flasks with the same medium (11 ml final volume) were inoculated to a final OD 600 0.03 and grown at 30° C., 250 RPM in a New Brunswick Scientific I24 shaker for 6 hours. Cultures were used to inoculate a final volume of 11 ml to an OD600 0.03 in 20 ml serum vials (Kimble Chase, Vineland, N.J.). The vials were sealed, and cultures grown at 30° C., 250 RPM in a New Brunswick Scientific I24 shaker for 38 hours. The cultures were sampled at 38 hours. Culture supernatants (collected using Spin-X centrifuge tube filter units, Costar Cat. No. 8169) were analyzed by HPLC (method described in U.S. Patent Appl. Pub. No. US 2007/0092957, incorporated by reference in its entirety) to determine the concentration of glucose and isobutanol.

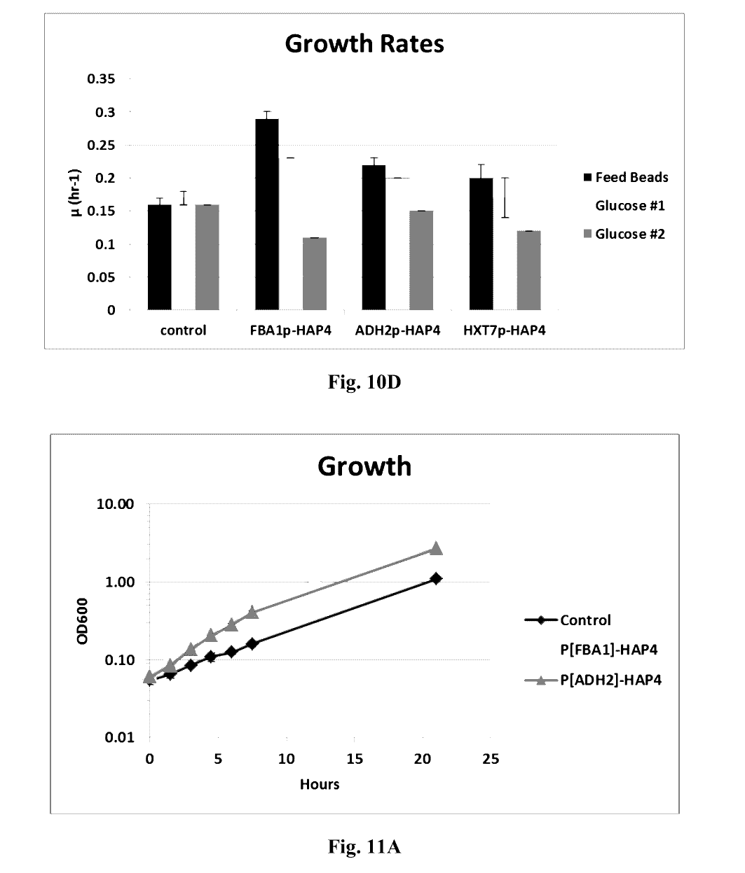

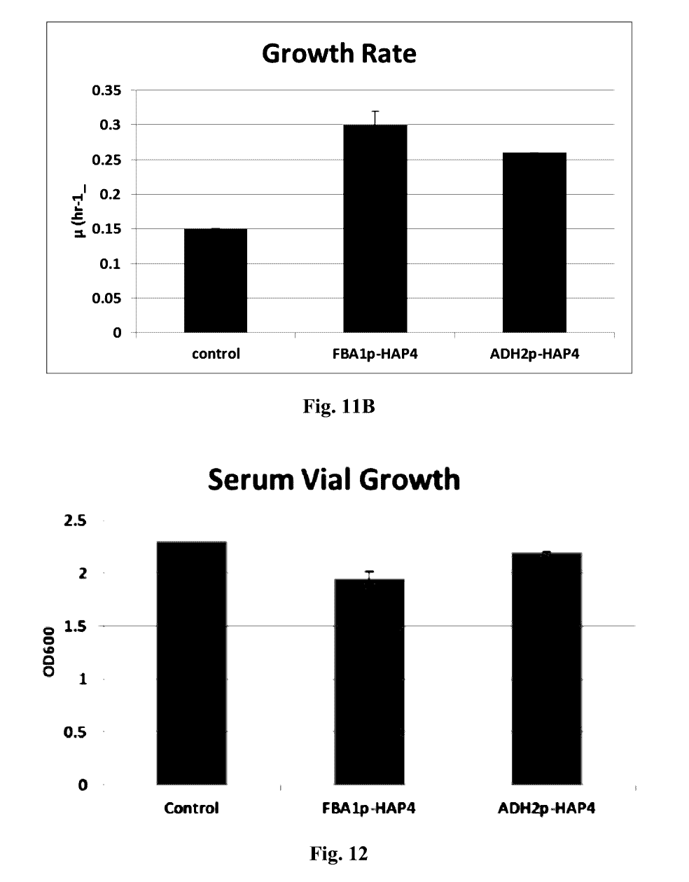

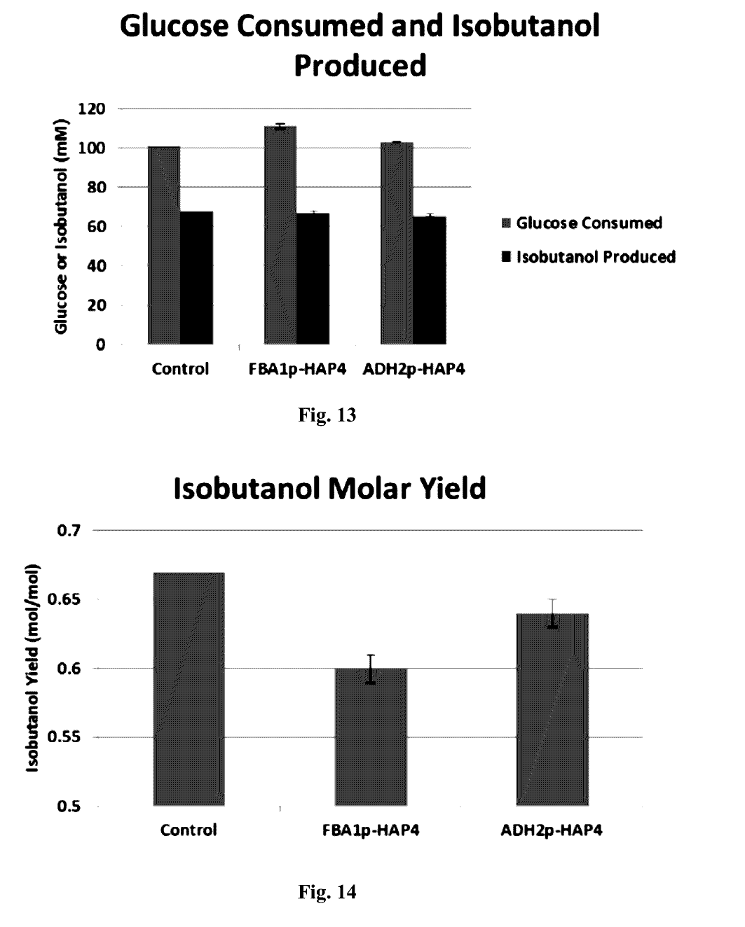

FIG. 12 shows growth of the strains in serum vials. FIG. 13 shows the amount of glucose consumed and isobutanol produced by the strains. FIG. 14 shows the isobutanol molar yield for the strains. The isobutanol molar yield for the strains with HAP4 expressed from the ADH2 promoter was higher than the strains with HAP4 expressed from the FBA1 promoter.

### Example 12

### Prophetic

### μcrit of Butanologen Yeast with and without Overexpression of Hap4

In ethanologen yeast μcrit represents a specific growth rate that is specific to each strain. If growth exceeds this growth rate lower biomass yields on glucose as compared to growth rates below μcrit are the result. It is generally assumed that μcrit correlates with the maximum specific respiratory capacity of the ethanologen yeast cell (Fiechter et al., Adv. Microb. Physiol. 22:123-183, 1981; see also specific rates of oxygen uptake (◯), carbon dioxide production () and cell yield (□) (gram [dry weight] gram of glucose⁻¹) as a function of the dilution rate in glucose-limited cultures of S. cerevisiae CBS 8066 in Postma et al., Appl. Environ. Microbiol. 55(2):468-477, 1989.). The specific growth rate in turn depends on the specific glucose uptake rate, so that μcrit may be understood to represent the specific glucose uptake rate after which the respiratory pathways are not able to cope with higher uptake fluxes, thus additional glucose is channeled into fermentative pathways to yield pathway intermediates as well as fermentation end products. Accordingly, other indicators of growth faster than μcrit may include (i) increased yields of fermentation products and pathway intermediates on glucose, as well as (ii) significantly increased RQ values.

This example is to demonstrate that butanologen yeast strains overexpressing Hap4 exhibit a higher μcrit as compared to control butanologen yeast strains. μcrit is determined in accelerostat experiments. One vial of frozen glycerol stock culture of each strain, PNY2145, PNY1650 and PNY1653, is inoculated into a 250 ml shake flask with 60 ml growth medium and additionally 100 mM MES buffer, pH 5.5. These seed cultures are incubated for 24 h at 30° C. and 250 rpm in an Innova Laboratory Shaker (New Brunswick Scientific, Edison, N.J.) until shortly before depletion of the carbon source.

The bioreactor experiments are carried out in 1 L Braun Biostat B+ fermenters (Sartorius, Goettingen, Germany). After sterilization of the bioreactors the vessels are filled with 450 ml of 0.1 M sterilized phosphate buffer, pH=5.5 (13.2 g/l monosodium phosphate monohydrate and 1.1 g/l disodium phosphate heptahydrate). Growth medium (Table 6) is prepared in 10 L glass bottles. The glucose solution is prepared separately, sterilized (20 min at 110° C. effectively), and added to the sterilized medium.

The experiment is started with addition of 50 ml of seed cultures. Simultaneously with the inoculation the feed pump is started at a constant flow of 40 ml/h to deliver growth medium to the fermenter. A second “harvest” pump is used to control the weight at 800 g. Consequently for approximately the first 8 h the working volume (V) of the fermenter is filling up from the approximately 500 ml at the start to 800 ml before the harvest pump is for the first time activated. Additional control points are pH=5.5 with 2 M KOH as a titrant, temperature at 30° C. Airflow is set to 0.8 standard liters per minute (SLPM), equivalent to 1 VVM at 800 ml culture volume. Stirrer is operated at a minimum of 500 rpm. Dissolved oxygen (DO) is monitored with oxygen probes in the fermenter. If DO drops below 20%, stirrer speed is increased in order to keep the DO at or above 20%.

In the first phase of the experiment a steady-state of the cultures at D=0.05 L/h is achieved. Steady-state of the cultures is characterized by constant (maximally about +/−5% variation) readings of the OD as well as CO₂ and O₂ concentration in the off-gas for at least 3 volume exchanges. Once the cultures are in steady-state, the process is switched from continuous culture mode into the accelerostat mode, i.e., a continuous culture setting with changing dilution rate. The accelerostat mode is achieved as the weight controller continues to maintain a constant volume of the fermenter of 800 ml but the dilution rates (D) in the fermenters are increased at a constant rate of 0.002 L/h per h. Increase of dilution rate at constant volume is accomplished through increasing the feed rate F of the pumps according to F=D*V

Samples are withdrawn at specific time points to allow for analysis of metabolite production and consumption in the medium. Metabolites may comprise but are not limited to acetate, ethanol, isobutanol, ketoisovalerate, isobutyric acid, isobutyraldehyde, acetoin, diacetyl, acetolactate, dihydroxyisovaleric acid, butanediol, pyruvate, malate, glucose, glycerol and the major 20 proteinogenic amino acids. One method applied to analyze compounds in supernatant is gas chromatography coupled to flame ionization detection (GC-FID). Another method applied to analyze compounds in supernatant is high performance liquid chromatography (HPLC). A BIO-RAD Aminex HPX-87H column was used in an isocratic method with 0.01 N sulfuric acid as eluent on a Waters Alliance 2695 Separations Module (Milford, Mass.). Flow rate is 0.60 ml/min, column temperature 40° C., injection volume 10 μl and run time 58 min. Detection is carried out with a refractive index detector (Waters 2414 RI) operated at 40° C. and an UV detector (Waters 2996 PDA) at 210 nm. Analysis of the major 20 proteinogenic amino acids is accomplished by ultra-pressure liquid chromatography (UPLC) and using the Waters AccQ•Fluor reagent kit (Waters, Milford, Mass.) with 6-aminoquinolyl-N-hydroxysuccinimidyl carbamate (AQC) as reactive reagent. Briefly, 70 μl of borate buffer is added into a HPLC sample vial and mixed with 10 μl of sample solution. Subsequently 20 μl of AccQ•Fluor reagent is added, the solution vortexed and heated in a heating block at 55° C. for 10 min. Separation and detection is carried out on a Waters UPLC Acquity system (Milford, Mass.) equipped with an AccQ-Tag Ultra 2.1×100 mm column. Mobile phase A is 10% AccQ-Tag Ultra eluent A, mobile phase B is AccQ-Tag Ultra eluent B, flow rate is 0.7 ml/min. Gradient is as follows: 0-5.74 min: 99.9% A, 0.1% B; 5.74-7.74 min: 90.9% A, 9.1% B; 7.74-8.04 min: 78.8% A, 21.2% B; 8.04-8.73 min: 40.4% A, 59.6% B; 8.73-9.5 min: 99.9% A, 0.1% B. Injection volume is 0.8 column temperature 60° C., total run time 9.5 min. Detection is accomplished with a PDA detector at 260 nm. Off-gas analysis is accomplished by a magnetic sector MS (Thermo Electron VG Prima δ B Process MS, Cheshire, UK). Compounds analyzed in the off-gas comprise but are not limited to N₂, H₂O, O₂, CO₂, isobutanol, isobutyraldehyde and ethanol. In addition, cell dry weight concentrations (CDW) in the fermentations are analyzed via optical density (OD) as well as cell dry weight measurement. OD is measured at λ=600 nm with an Ultrospec 3000 spectrophotometer (Pharmacia Biotech, Piscataway, N.J.). Cell dry weight is determined with a filter method. Briefly, for the filter method 0.2 μm filters are dried at 80° C. in an oven and subsequently weighed. Next, defined volumes of cell culture are filtered through the pre-weighed filters. The filters with cake are washed twice with distilled water, dried at 80° C. in an oven until constant weight, and weighed again. The difference in the weight with knowledge of the filtered sample volume allows for the determination of CDW.

Knowledge of CDW, OD and metabolite measurements allows for the determination of specific production and consumption rates as well as yields of metabolites and biomass. Based on the specific production and consumption rates as well as yields μcrit of the strains is determined. μcrit is the specific growth rate that represents an inflection point of biomass growth yield, beyond which an increase in fermentation products and excreted intermediates as well as RQ is detected, but without significant further increase in the specific oxygen uptake rate (qO₂), despite further increase in growth rate is observed. It is found that strains PNY1650 and PNY1653 exhibit higher values of μcrit than strain PNY2145.

TABLE 6
Composition of growth medium

| | | | amount |
|---|---|---|---|
| component [ ] | MW [g/mol] | volume [ml] | [g] |
| ammonium sulphate | 132.14 | — | 12.5 |
| potassiumdihydrogen phosphate | 136.09 | — | 7.50 |
| magnesium sulfate•7H₂O | 246.47 | — | 1.25 |
| trace element solution (Table 7) | — | 2.50 | — |
| vitamin solution (Table 8) | — | 2.50 | — |
| nicotinic acid | — | — | 0.02 |
| thiamine | — | — | 0.02 |
| silicone antifoaming agent | — | 0.05 | — |
| glucose | — | — | 20.00 |
| H₂O demineralized | — | ad 1000 | — |

TABLE 7
Trace element solution

| | | MW | amount | volume |
|---|---|---|---|---|
| Compound [ ] | formula [ ] | [g/mol] | [g] | [ml] |
| EDTA | C₁₀H₁₄N₂Na₂O₈•2H₂O | 372.24 | 15.00 | — |
| zinc sulphate heptahydrate | ZnSO₄•7H₂O | 287.54 | 4.50 | — |
| manganese chloride dihydrate | MnCl₂•2H₂O | 161.88 | 0.84 | — |
| cobalt(II)chloride hexahydrate | CoCl₂•6H₂O | 237.93 | 0.30 | — |
| copper(II)sulphate pentahydrate | CuSO₄•5H₂O | 249.68 | 0.30 | — |
| di-sodium molybdenum | Na₂MoO₄•2H₂O | 241.95 | 0.40 | — |
| dihydrate | | | | |
| calcium chloride dihydrate | CaCl₂•2H₂O | 147.02 | 4.50 | — |
| iron sulphate heptahydrate | FeSO₄•7H₂O | 278.02 | 3.00 | — |
| boric acid | H₃BO₃ | 61.83 | 1.00 | — |
| potassium iodide | KI | 166.01 | 0.10 | — |
| demineralized water | — | — | — | ad 1000 |

TABLE 8
Vitamin solution

| | | MW | amount | volume |
|---|---|---|---|---|
| compound [ ] | Formula [ ] | [g/mol] | [g] | [ml] |
| biotin (D−) | C₁₀H₁₆N₂O₃S | 244.31 | 0.05 | — |
| Ca D(+) | C₁₈H₃₂CaN₂O₁₀ | 476.54 | 1.00 | — |
| panthotenate | | | | |
| nicotinic acid | C₆H₅NO₂ | 123.11 | 1.00 | — |
| myo-inositol | C₆H₁₂O₆ | 180.16 | 25.00 | — |
| thiamine chloride | C₁₂H₁₇ClN₄OS•HCL | 337.27 | 1.00 | — |
| hydrochloride | | | | |
| pyridoxol | C₈H₁₂ClNO₃ | 205.64 | 1.00 | — |
| hydrochloride | | | | |
| p-aminobenzoic acid | C₇H₇NO₂ | 137.14 | 0.20 | — |
| demineralized water | — | — | — | ad 1000 |

### Example 13

### Prophetic

### Glucose Limited Fed-Batch with Exponential Feeding Profile

This example demonstrates the improved productivity of butanologen yeast overexpressing Hap4 as compared to control butanologen yeast in an aerobic, glucose limited fed-batch with exponential feeding profile. One vial of frozen glycerol stock culture of each strain, PNY2145 and PNY1650, are inoculated into a 1 L shake flask each with 250 mL seed medium. The cultures are incubated at 30° C. and 250 rpm in an Innova Laboratory Shaker (New Brunswick Scientific, Edison, N.J.) until optical density (OD) of the cultures exceeds 1.000. OD is measured at λ=600 nm with an Ultrospec 3000 spectrophotometer (Pharmacia Biotech, Piscataway, N.J.). The seed medium contains per liter: KH₂PO₄: 10.0 g, MgSO₄: 2.5 g and 10 mL of trace element solution (Table 7). After autoclaving (121° C., 20 min), filter-sterilized urea and vitamin solution (Table 8) are added to a final concentration of 3.0 g/L and 15 ml/L, respectively. Glucose is added separately to a final concentration of 20 g/L after sterilization at 110° C. for 20 min. Initial pH of the seed medium is 5.0. The trace element solution contains per liter: EDTA, 15 g; ZnSO₄, 5.75 g; MnCl₂, 0.32 g; CuSO₄, 0.50 g; CoCl₂, 0.47 g; Na₂MoO₄, 0.48 g; CaCl₂, 2.9 g; FeSO₄, 2.8 g. The trace element solution is sterilized at 121° C. for 20 min. The vitamin solution contains per liter: biotin, 0.05 g; calcium pantothenate, 1.0 g; nicotinic acid, 1.0 g; myoinositol, 25.0 g; thiamine hydrochloride, 1 g; pyridoxol hydrochloride, 1 g; p-aminobenzoic acid, 0.2 g. The vitamin solution is filter-sterilized before use.

The fed-batch cultivations are carried out in 2 L bioreactors (Sartorius Biostat B-DCU Twin 2L, (Sartorius, Goettingen, Germany)) with a starting volume of 1 L. For the initial batch phase a startup medium comprising 748 ml of demineralized water, 15.0 g (NH₄)₂SO₄, 8.0 g KH₂PO₄, 3.0 g MgSO₄, 10 ml of trace element solution, 0.3 ml of anti-foaming agent Struktol J673 and 0.4 g of ZnSO₄ is prepared, sterilized in an autoclave at 121° C. for 45 min, and added to the previously sterilized bioreactor. Subsequently 12 mL of vitamin solution and 40 ml of sterilized glucose solution with 250 g/l glucose are added. The batch phase of the process is initiation with the addition of 200 ml of the inoculum cultures. Temperature is controlled at 30° C., pH at 5.0 with a 14.7 mM ammonium hydroxide solution. The dissolved-oxygen concentration is continuously measured with a polarographic oxygen electrode (Hamilton Oxyferm FDA 225, NV, USA), and kept above 20% of air saturation at a constant impeller speed of 1500 rpm. The air flow is maintained at 0.5 L/h-1 with internal Sartorius mass flow meter (Sartorius Biostat B-DCU, NY, USA). The exhaust gas is cooled in a condenser (12° C.). O₂, CO₂ and N₂ concentrations in the off-gas are monitored with mass spectrometer (Thermo Electron VG Prima δ B Process MS, Cheshire, UK).

Shortly before the glucose in the medium is exhausted the feed pumps are started and the fed-batch phase is initiated. The feed medium for the fed-batch phase contains per liter: KH₂PO₄: 9.0 g, MgSO₄: 2.5 g; K₂SO₄: 3.5 g; Na₂SO₄: 0.28 g, glucose: 500 g and 10 ml of trace-element solution. After sterilization of the medium at 110° C. for 20 min also 12 mL/L of vitamin solution is added. The medium is pumped into the reactor using a controllable peristaltic pump (SciLog, Tandem model 1081, WI, USA).

The flow rate of the exponential feed in dependence of the time is calculated according to

$$F = \frac{μ \cdot Xo \cdot Vo}{Si \cdot Yx / s} \cdot {e}^{μ \cdot t}$$

whereas F denotes the flow rate of the medium feed [L/h], Yx/s the biomass yield on substrate in the current feeding regime [g(CDW)/g(glucose)], Xo the biomass concentration at the start of the fed-batch phase [g(CDW)/l], Vo the working volume of the culture at the start of the fed-batch phase culture [l], Si the glucose concentration in the feed [g(glucose)/l), and t the elapsed time after starting the feed [h]. In both cultivations the exponent of the exponential feeding profile is set to the μcrit of PNY1650 (determined as described in an example previously). The amount of medium added during the fed-batch phase is recorded by continuous monitoring of the mass of the reservoir vessels by electronic balances.

During the experiment samples are withdrawn at specific time points to allow for analysis of extracellular compound production and consumption in the medium. Metabolites may comprise but are not limited to acetate, ethanol, isobutanol, ketoisovalerate, isobutyric acid, isobutyraldehyde, acetoin, diacetyl, dihydroxyisovaleric acid, butanediol, pyruvate, malate, glucose and glycerol. Extracellular compound analysis in supernatant is accomplished by HPLC. A BIO-RAD Aminex HPX-87H column was used in an isocratic method with 0.01 N sulfuric acid as eluent on a Waters Alliance 2695 Separations Module (Milford, Mass.). Flow rate is 0.60 ml/min, column temperature 40° C., injection volume 10 μl and run time 58 min. Detection is carried out with a refractive index detector (Waters 2414 RI) operated at 40° C. and an UV detector (Waters 2996 PDA) at 210 nm. Biomass growth is monitored in determining optical density (OD) and cell dry weight (CDW). OD is measured at λ=600 nm with an Ultrospec 3000 spectrophotometer (Pharmacia Biotech, Piscataway, N.J.). For cell dry weight determination 5 ml of culture samples are centrifuged in pre-weighed 15 mL round bottom centrifuge tubes (Kimble HS 45500-15, Thermo Fisher Scientific, NH, US) at 5000 rpm for 10 min using a high speed centrifuge (Eppendorf 5804R, NY, USA). The supernatant is decanted and the pellets washed with 5 mL of distilled water. After repeated centrifugation and decanting the pellet is dried at 80° C. in an oven until constant weight.

Both fermentations are stopped at the same run time the moment in one of the cultivations no significant increase of biomass is observed. Cultivation of PNY1650 shows higher biomass productivity than cultivation of PNY2145. Cultivation of PNY1650 shows higher biomass yield on glucose than cultivation of PNY2145.

### Example 14

### Prophetic

### Glucose Limited Fed-Batch with Exponential Feeding Profile

This example demonstrates the improved productivity of butanologen yeast overexpressing Hap4 as compared to control butanologen yeast in an aerobic, glucose limited fed-batch with exponential feeding profile. One vial of frozen glycerol stock culture of each strain, PNY2145 and PNY1653, are inoculated into a 1 L shake flask each with 250 mL seed medium. The cultures are incubated at 30° C. and 250 rpm in an Innova Laboratory Shaker (New Brunswick Scientific, Edison, N.J.) until optical density (OD) of the cultures exceeds 1.000. OD is measured at λ=600 nm with an Ultrospec 3000 spectrophotometer (Pharmacia Biotech, Piscataway, N.J.). The seed medium contains per liter: KH₂PO₄: 10.0 g, MgSO₄: 2.5 g and 10 mL of trace element solution. After autoclaving (121° C., 20 min), filter-sterilized urea and vitamin solution are added to a final concentration of 3.0 g/L and 15 ml/L, respectively. Glucose is added separately to a final concentration of 20 g/L after sterilization at 110° C. for 20 min. Initial pH of the seed medium is 5.0. The trace element solution contains per liter: EDTA, 15 g; ZnSO₄, 5.75 g; MnCl₂, 0.32 g; CuSO₄, 0.50 g; CoCl₂, 0.47 g; Na₂MoO₄, 0.48 g; CaCl₂, 2.9 g; FeSO₄, 2.8 g. The trace element solution is sterilized at 121° C. for 20 min. The vitamin solution contains per liter: biotin, 0.05 g; calcium pantothenate, 1.0 g; nicotinic acid, 1.0 g; myoinositol, 25.0 g; thiamine hydrochloride, 1 g; pyridoxol hydrochloride, 1 g; p-aminobenzoic acid, 0.2 g. The vitamin solution is filter-sterilized before use.

The fed-batch cultivations are carried out in 2 L bioreactors (Sartorius Biostat B-DCU Twin 2L, NY, USA) with a starting volume of 1 L. For the initial batch phase a startup medium comprising 748 ml of demineralized water, 15.0 g (NH₄)₂SO₄, 8.0 g KH₂PO₄, 3.0 g MgSO₄, 10 ml of trace element solution, 0.3 ml of anti-foaming agent Struktol J673 and 0.4 g of ZnSO₄ is prepared, sterilized in an autoclave at 121° C. for 45 min, and added to the previously sterilized bioreactor. Subsequently 12 mL of vitamin solution and 40 ml of sterilized glucose solution with 250 g/l glucose are added. The batch phase of the process is initiated with the addition of 200 ml of the inoculum cultures. Temperature is controlled at 30° C., pH at 5.0 with a 14.7 mM ammonium hydroxide solution. The dissolved-oxygen concentration is continuously measured with a polarographic oxygen electrode (Hamilton Oxyferm FDA 225, NV, USA) and kept above 20% of air saturation at a constant impeller speed of 1500 rpm. The air flow is maintained at 0.5 L/h-1 with internal Sartorius mass flow meter (Sartorius Biostat B-DCU, NY, USA). The exhaust gas is cooled in a condenser (12° C.). O₂, CO₂ and N₂ concentrations in the off-gas are monitored with mass spectrometer (Thermo Electron VG Prima δ B Process MS, Cheshire, UK).

Shortly before the glucose in the medium is exhausted the feed pumps are started and the fed-batch phase is initiated. The feed medium for the fed-batch phase contains per liter: KH₂PO₄: 9.0 g, MgSO₄: 2.5 g; K₂SO₄: 3.5 g; Na₂SO₄: 0.28 g, glucose: 500 g and 10 ml of trace-element solution. After sterilization of the medium at 110° C. for 20 min also 12 mL/L of vitamin solution is added. The medium is pumped into the reactor using a controllable peristaltic pump (SciLog, Tandem model 1081, WI, USA).

The flow rate of the exponential feed in dependence of the time is calculated according to

$$F = \frac{μ \cdot Xo \cdot Vo}{Si \cdot Yx / s} \cdot {e}^{μ \cdot t}$$

whereas F denotes the flow rate of the medium feed [L/h], Yx/s the biomass yield on substrate in the current feeding regime [g(CDW)/g(glucose)], Xo the biomass concentration at the start of the fed-batch phase [g(CDW)/l], Vo the working volume of the culture at the start of the fed-batch phase culture [l], Si the glucose concentration in the feed [g(glucose)/l), and t the elapsed time after starting the feed [h]. In both cultivations the exponent of the exponential feeding profile is set to the μcrit of PNY1653 (determined as described in an example previously). The amount of medium added during the fed-batch phase is recorded by continuous monitoring of the mass of the reservoir vessels by electronic balances.

During the experiment samples are withdrawn at specific time points to allow for analysis of extracellular compound production and consumption in the medium. Metabolites of interest comprise but are not limited to acetate, ethanol, isobutanol, ketoisovalerate, isobutyric acid, isobutyraldehyde, acetoin, diacetyl, dihydroxyisovaleric acid, butanediol, pyruvate, malate, glucose and glycerol. Extracellular compound analysis in supernatant is accomplished by HPLC. A BIO-RAD Aminex HPX-87H column was used in an isocratic method with 0.01 N sulfuric acid as eluent on a Waters Alliance 2695 Separations Module (Milford, Mass.). Flow rate is 0.60 ml/min, column temperature 40° C., injection volume 10 μl and run time 58 min. Detection is carried out with a refractive index detector (Waters 2414 RI) operated at 40° C. and an UV detector (Waters 2996 PDA) at 210 nm. Biomass growth is monitored in determining optical density (OD) and cell dry weight (CDW). OD is measured at λ=600 nm with an Ultrospec 3000 spectrophotometer (Pharmacia Biotech, Piscataway, N.J.). For cell dry weight determination 5 ml of culture samples are centrifuged in pre-weighed 15 mL round bottom centrifuge tubes (Kimble HS 45500-15, Thermo Fisher Scientific, NH, US) at 5000 rpm for 10 min using a high speed centrifuge (Eppendorf 5804R, NY, USA). The supernatant is decanted and the pellets washed with 5 mL of distilled water. After repeated centrifugation and decanting the pellet is dried at 80° C. in an oven until constant weight is achieved.

Both fermentations are stopped at the same run time the moment no significant increase of biomass is observed in one of the cultivations. Cultivation of PNY1653 shows higher biomass productivity than that of PNY2145. Cultivation of PNY1653 shows higher biomass yield on glucose than that of PNY2145.

1. A recombinant yeast cell, comprising (a) a recombinant polynucleotide encoding a gene for a subunit of the HAP transcriptional complex; and (b) an engineered isobutanol biosynthetic pathway.

2. The recombinant yeast cell of claim 1, wherein the subunit is Hap2, Hap3, Hap4 or Hap5.

3. The recombinant yeast cell of claim 1, wherein the subunit comprises an amino acid sequence at least 80%, at least 85%, at least 90%, at least 95%, at least 96%, at least 97%, at least 98%, or at least 99% identical to any of SEQ ID NOs: 2, 4, 6, or 8.

4. The recombinant yeast cell of claim 1, wherein the polynucleotide comprises a nucleic acid sequence at least 80%, at least 85%, at least 90%, at least 95%, at least 96%, at least 97%, at least 98% or at least 99% identical to any of SEQ ID NOs: 1, 3, 5, or 7.

5. The recombinant yeast cell of claim 1, wherein the gene is expressed during propagation phase of a fermentation-based production process.

6. The recombinant yeast cell of claim 1, wherein the gene is down-regulated or not expressed during production phase of a fermentation-based production process.

7. The recombinant yeast cell of claim 1, wherein the gene is operably linked to a conditional promoter.

8. (canceled)

9. The recombinant yeast cell of claim 7, wherein the conditional promoter is ADH2, HXT5 or HXT7.

10. The recombinant yeast cell of claim 7, wherein the conditional promoter comprises a nucleic acid sequence at least 80%, at least 85%, at least 90%, at least 95%, at least 96%, at least 97%, at least 98% or at least 99% identical to any of SEQ ID NOs:74-85.

11. The recombinant yeast cell of claim 1, further comprising one or more genetic modifications selected from at least one genetic modification that reduces or eliminates activity of an endogenous pyruvate decarboxylase and at least one genetic modification that reduces or eliminates activity of an endogenous glycerol-3-phosphate dehydrogenase.

12. (canceled)

13. (canceled)

14. (canceled)

15. The recombinant yeast cell of claim 1, wherein the isobutanol biosynthetic pathway comprises one or more of (a) at least one genetic construct encoding an acetolactate synthase; (b) at least one genetic construct encoding acetohydroxy acid isomeroreductase; (c) at least one genetic construct encoding acetohydroxy acid dehydratase; (d) at least one genetic construct encoding branched-chain keto acid decarboxylase; and (e) at least one genetic construct encoding branched-chain alcohol dehydrogenase.

16. The recombinant yeast cell of claim 1, wherein the yeast is from the genus Saccharomyces, Schizosaccharomyces, Hansenula, Kluyveromyces, Candida, Pichia, or Yarrowia.

17. (canceled)

18. (canceled)

19. (canceled)

20. A method for increasing growth rate of a yeast cell, comprising introducing into a yeast cell (a) a recombinant polynucleotide encoding a gene for a subunit of the HAP transcriptional complex, and (b) an engineered higher alcohol biosynthetic pathway; wherein growth rate of the yeast cell during fermentation-based production process is greater when compared to growth rate of a yeast cell that does not contain a recombinant polynucleotide encoding a gene for a subunit of the HAP transcriptional complex.

21. (canceled)

22. (canceled)

23. The method of claim 20, wherein the gene is expressed during propagation phase of the fermentation-based production process.

24. The method of claim 20, wherein the gene is down-regulated or not expressed during production phase of the fermentation-based production process.

25. The method of claim 20, wherein the gene is operably linked to a conditional promoter.

26. (canceled)

27. The method of claim 25, wherein the conditional promoter is ADH2, HXT5 or HXT7.

28. The method of claim 25, wherein the conditional promoter comprises a nucleic acid sequence at least 80%, at least 85%, at least 90%, at least 95%, at least 96%, at least 97%, at least 98% or at least 99% identical to any of SEQ ID NOs:74-85.

29. (canceled)

30. (canceled)

31. (canceled)

32. The method of claim 20, wherein ethanol or sodium acetate is present during the fermentation-based production process.

33. (canceled)

34. A method for production of isobutanol, comprising (a) providing a recombinant yeast cell of claim 1; and (b) culturing the cell of (a) under conditions wherein isobutanol is produced.

35. The method of claim 34, further comprising (c) recovering the isobutanol.

36. (canceled)

37. (canceled)

38. (canceled)
